SEQUENCE LISTING (184 sequences)

SEQ ID NO: 1 - protein, 5 aa, synthetic construct
  REGION 1..5: note = Synthetic
SYGIS

SEQ ID NO: 2 - protein, 17 aa, synthetic construct
  REGION 1..17: note = Synthetic
  VARIANT 6: note = Xaa is Ser, Asn, or Gly
  VARIANT 7: note = Xaa is is Gly or Ser
  VARIANT 8: note = Xaa is or Gly, Asn or Ser
  VARIANT 9: note = Xaa is Thr or Ala
WISAYXXXXNYAQKLQG

SEQ ID NO: 3 - protein, 14 aa, synthetic construct
  REGION 1..14: note = Synthetic
DLFPTIFGVSYYYY

SEQ ID NO: 4 - skipped

SEQ ID NO: 5 - protein, 4 aa, synthetic construct
  REGION 1..4: note = Synthetic
SGGT

SEQ ID NO: 6 - protein, 4 aa, synthetic construct
  REGION 1..4: note = Synthetic
NSNT

SEQ ID NO: 7 - protein, 4 aa, synthetic construct
  REGION 1..4: note = Synthetic
GGST

SEQ ID NO: 8 - protein, 4 aa, synthetic construct
  REGION 1..4: note = Synthetic
SGNA

SEQ ID NO: 9 - protein, 11 aa, synthetic construct
  REGION 1..11: note = Synthetic
  VARIANT 7: note = Xaa is Gly or Ser
  VARIANT 8: note = Xaa is Asn or Gly
RASQSIXXRLA

SEQ ID NO: 10 - protein, 7 aa, synthetic construct
  REGION 1..7: note = Synthetic
  VARIANT 4: note = Xaa is Thr or Asn
  VARIANT 5: note = Xaa is Ser or Leu
  VARIANT 7: note = Xaa is Thr or Ser
EASXXEX

SEQ ID NO: 11 - skipped

SEQ ID NO: 12 - protein, 14 aa, synthetic construct
  REGION 1..14: note = Synthetic
  VARIANT 12: note = Xaa is Tyr or Ser
TGTSSDVGGYNXVS

SEQ ID NO: 13 - protein, 7 aa, synthetic construct
  REGION 1..7: note = Synthetic
EVTNRPS

SEQ ID NO: 14 - protein, 11 aa, synthetic construct
  REGION 1..11: note = Synthetic
SSFKRGSTLVV

SEQ ID NO: 15 - protein, 11 aa, synthetic construct
  REGION 1..11: note = Synthetic
RASQSIGNRLA

SEQ ID NO: 16 - protein, 7 aa, synthetic construct
  REGION 1..7: note = Synthetic
EASTSET

SEQ ID NO: 17 - protein, 9 aa, synthetic construct
  REGION 1..9: note = Synthetic
QQSYSTPYT

SEQ ID NO: 18 - protein, 17 aa, synthetic construct
  REGION 1..17: note = Synthetic
WISAYSGGTNYAQKLQG

SEQ ID NO: 19 - protein, 11 aa, synthetic construct
  REGION 1..11: note = Synthetic
RASQSISGRLA

SEQ ID NO: 20 - protein, 7 aa, synthetic construct
  REGION 1..7: note = Synthetic
EASNLES

SEQ ID NO: 21 - protein, 9 aa, synthetic construct
  REGION 1..9: note = Synthetic
QQSYSWPRT

SEQ ID NO: 22 - protein, 10 aa, synthetic construct
  REGION 1..10: note = Synthetic
QQSYSTPRVT

SEQ ID NO: 23 - protein, 17 aa, synthetic construct
  REGION 1..17: note = Synthetic
WISAYNSNTNYAQKLQG

SEQ ID NO: 24 - protein, 10 aa, synthetic construct
  REGION 1..10: note = Synthetic
QQANTFPRVS

SEQ ID NO: 25 - protein, 14 aa, synthetic construct
  REGION 1..14: note = Synthetic
TGTSSDVGGYNYVS

SEQ ID NO: 26 - protein, 14 aa, synthetic construct
  REGION 1..14: note = Synthetic
TGTSSDVGGYNSVS

SEQ ID NO: 27 - protein, 123 aa, synthetic construct
  REGION 1..123: note = Synthetic
EVQLVQSGAEVKRPGASVKVSCKASGYPFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSS

SEQ ID NO: 28 - protein, 107 aa, synthetic construct
  REGION 1..107: note = Synthetic
DIQMTQSPSTLSASVRDRVIITCRASQSIGNRLAWYQHKPGKAPKLLIYEASTSETGVPSRFSGSGSGTDFTLTISSLQPEDFATYYCQQSYSTPYTFGQGTKLEIK

SEQ ID NO: 29 - protein, 123 aa, synthetic construct
  REGION 1..123: note = Synthetic
EVQLVQSGAEVKRPGASVKVSCKASGYTFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSS

SEQ ID NO: 30 - protein, 107 aa, synthetic construct
  REGION 1..107: note = Synthetic
DIQMTQSPSTLSASVGDRVTITCRASQSISGRLAWYQQKPGKAPNLLIYEASNLESGVPSRFSGSGSGTEFTLTISSLQPEDFATYYCQQSYSWPRTFGQGTKVEIK

SEQ ID NO: 31 - protein, 123 aa, synthetic construct; identical to SEQ ID NO: 29
  REGION 1..123: note = Synthetic

SEQ ID NO: 32 - protein, 108 aa, synthetic construct
  REGION 1..108: note = Synthetic
DIQMTQSPSTLSASVGDRVTITCRASQSISGRLAWYQQKPGKAPNLLIYEASNLESGVPSRFSGSGSGTEFTLTISSLQPEDFATYYCQQSYSTPRVTFGQGTKVEIK

SEQ ID NO: 33 - protein, 123 aa, synthetic construct
  REGION 1..123: note = Synthetic
EVQLVQSGAEVKRPGASVKVSCKASGYPFTSYGISWVRQAPGQGLEWMGWISAYNSNTNYAQKLQGRVTMTTDTSTSTAYMELRSLRSDDTAVYYCARDLFPTIFGVSYYYYWGQGTLVTVSS

SEQ ID NO: 34 - protein, 107 aa, synthetic construct; identical to SEQ ID NO: 28
  REGION 1..107: note = Synthetic

SEQ ID NO: 35 - protein, 123 aa, synthetic construct
  REGION 1..123: note = Synthetic
EVQLVQSGAEVKRPGASVKVSCKASGYTFTSYGISWVRQAPGQGLEWMGWISAYNSNTNYAQKLQGRVTMTTDTSTSTAYMELRSLRSDDTAVYYCARDLFPTIFGVSYYYYWGQGTLVTVSS

SEQ ID NO: 36 - protein, 107 aa, synthetic construct; identical to SEQ ID NO: 30
  REGION 1..107: note = Synthetic

SEQ ID NO: 37 - protein, 123 aa, synthetic construct; identical to SEQ ID NO: 35
  REGION 1..123: note = Synthetic

SEQ ID NO: 38 - protein, 108 aa, synthetic construct; identical to SEQ ID NO: 32
  REGION 1..108: note = Synthetic

SEQ ID NO: 39 - protein, 123 aa, synthetic construct; identical to SEQ ID NO: 35
  REGION 1..123: note = Synthetic

SEQ ID NO: 40 - protein, 108 aa, synthetic construct
  REGION 1..108: note = Synthetic
DIQMTQSPSTLSASVGDRVTITCRASQSISGRLAWYQQKPGKAPNLLIYEASNLESGVPSRFSGSGSGTEFTLTINSLQPDDFATYYCQQANTFPRVSFGGGTKVEIK

SEQ ID NO: 41 - protein, 123 aa, synthetic construct; identical to SEQ ID NO: 29
  REGION 1..123: note = Synthetic

SEQ ID NO: 42 - protein, 111 aa, synthetic construct
  REGION 1..111: note = Synthetic
QSALTQPASVSGSPGQSITISCTGTSSDVGGYNYVSWYQQFPGKAPKLMIFEVTNRPSGVSDRFSGSKSDNTASLTISGLQAEDEAEYYCSSFKRGSTLVVFGGGTKLTVL

SEQ ID NO: 43 - protein, 123 aa, synthetic construct; identical to SEQ ID NO: 35
  REGION 1..123: note = Synthetic

SEQ ID NO: 44 - protein, 111 aa, synthetic construct
  REGION 1..111: note = Synthetic
QSALTQPASVSGSPGQSITISCTGTSSDVGGYNSVSWYQQFPGKAPKLMIFEVTNRPSGVSDRFSGSKSDNTASLTISGLQAEDEAEYYCSSFKRGSTLVVFGGGTKLTVL

SEQ ID NO: 45 - protein, 123 aa, synthetic construct
  REGION 1..123: note = Synthetic
EVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDLFPTIFGVSYYYYWGQGTLVTVSS

SEQ ID NO: 46 - protein, 111 aa, synthetic construct; identical to SEQ ID NO: 44
  REGION 1..111: note = Synthetic

SEQ ID NO: 47 - protein, 452 aa, synthetic construct
  REGION 1..452: note = Synthetic
EVQLVQSGAEVKRPGASVKVSCKASGYPFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 214 aa, synthetic construct
  REGION 1..214: note = Synthetic
DIQMTQSPSTLSASVRDRVIITCRASQSIGNRLAWYQHKPGKAPKLLIYEASTSETGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 452 aa, synthetic construct
  REGION 1..452: note = Synthetic
EVQLVQSGAEVKRPGASVKVSCKASGYTFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 214 aa, synthetic construct
  REGION 1..214: note = Synthetic
DIQMTQSPSTLSASVGDRVTITCRASQSISGRLAWYQQKPGKAPNLLIYEASNLESGVPSRFSGSGSGTEFTLTISSLQPEDFATYYCQQSYSWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 452 aa, synthetic construct; identical to SEQ ID NO: 49
  REGION 1..452: note = Synthetic

SEQ ID NO: 52 - protein, 215 aa, synthetic construct
  REGION 1..215: note = Synthetic
DIQMTQSPSTLSASVGDRVTITCRASQSISGRLAWYQQKPGKAPNLLIYEASNLESGVPSRFSGSGSGTEFTLTISSLQPEDFATYYCQQSYSTPRVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 452 aa, synthetic construct
  REGION 1..452: note = Synthetic
EVQLVQSGAEVKRPGASVKVSCKASGYPFTSYGISWVRQAPGQGLEWMGWISAYNSNTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 215 aa, synthetic construct
  REGION 1..215: note = Synthetic
DIQMTQSPSTLSASVRDRVIITCRASQSIGNRLAWYQHKPGKAPKLLIYEASTSETGVPSRFSGSGSGTDFTLTISSLQPEDFATYYCQQSYSTPYTFGQGTKLEIKRTAAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 55 - protein, 452 aa, synthetic construct
  REGION 1..452: note = Synthetic
EVQLVQSGAEVKRPGASVKVSCKASGYTFTSYGISWVRQAPGQGLEWMGWISAYNSNTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 215 aa, synthetic construct
  REGION 1..215: note = Synthetic
DIQMTQSPSTLSASVGDRVTITCRASQSISGRLAWYQQKPGKAPNLLIYEASNLESGVPSRFSGSGSGTEFTLTISSLQPEDFATYYCQQSYSWPRTFGQGTKVEIKRTAAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 57 - protein, 452 aa, synthetic construct; identical to SEQ ID NO: 55
  REGION 1..452: note = Synthetic

SEQ ID NO: 58 - protein, 216 aa, synthetic construct
  REGION 1..216: note = Synthetic
DIQMTQSPSTLSASVGDRVTITCRASQSISGRLAWYQQKPGKAPNLLIYEASNLESGVPSRFSGSGSGTEFTLTISSLQPEDFATYYCQQSYSTPRVTFGQGTKVEIKRTAAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 59 - protein, 452 aa, synthetic construct; identical to SEQ ID NO: 55
  REGION 1..452: note = Synthetic

SEQ ID NO: 60 - protein, 216 aa, synthetic construct
  REGION 1..216: note = Synthetic
DIQMTQSPSTLSASVGDRVTITCRASQSISGRLAWYQQKPGKAPNLLIYEASNLESGVPSRFSGSGSGTEFTLTINSLQPDDFATYYCQQANTFPRVSFGGGTKVEIKRTAAAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 61 - protein, 452 aa, synthetic construct; identical to SEQ ID NO: 49
  REGION 1..452: note = Synthetic

SEQ ID NO: 62 - protein, 217 aa, synthetic construct
  REGION 1..217: note = Synthetic
QSALTQPASVSGSPGQSITISCTGTSSDVGGYNYVSWYQQFPGKAPKLMIFEVTNRPSGVSDRFSGSKSDNTASLTISGLQAEDEAEYYCSSFKRGSTLVVFGGGTKLTVLGQPAAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 63 - protein, 452 aa, synthetic construct; identical to SEQ ID NO: 55
  REGION 1..452: note = Synthetic

SEQ ID NO: 64 - protein, 217 aa, synthetic construct
  REGION 1..217: note = Synthetic
QSALTQPASVSGSPGQSITISCTGTSSDVGGYNSVSWYQQFPGKAPKLMIFEVTNRPSGVSDRFSGSKSDNTASLTISGLQAEDEAEYYCSSFKRGSTLVVFGGGTKLTVLGQPAAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - protein, 452 aa, synthetic construct
  REGION 1..452: note = Synthetic
EVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 217 aa, synthetic construct; identical to SEQ ID NO: 64
  REGION 1..217: note = Synthetic

SEQ ID NO: 67 - protein, 8 aa, synthetic construct
  REGION 1..8: note = Synthetic
  VARIANT 3: note = Xaa is Pro or Thr
GYXFTSYG

SEQ ID NO: 68 - protein, 8 aa, synthetic construct
  REGION 1..8: note = Synthetic
  VARIANT 5: note = Xaa is Ser, Asn or Gly
  VARIANT 6: note = Xaa is Gly or Ser
  VARIANT 7: note = Xaa is Gly, Asn or Ser
  VARIANT 8: note = Xaa is Thr or Ala
ISAYXXXX

SEQ ID NO: 69 - protein, 16 aa, synthetic construct
  REGION 1..16: note = Synthetic
ARDLFPTIFGVSYYYY

SEQ ID NO: 70 - protein, 6 aa, synthetic construct
  REGION 1..6: note = Synthetic
  VARIANT 4: note = Xaa is Gly or Ser
  VARIANT 5: note = Xaa is Asn or Gly
QSIXXR

SEQ ID NO: 71 - skipped

SEQ ID NO: 72 - protein, 9 aa, synthetic construct
  REGION 1..9: note = Synthetic
  VARIANT 3: note = Xaa is Ser or Ala
  VARIANT 4: note = Xaa is Tyr or Asn
  VARIANT 5: note = Xaa is Ser or Thr
QQXXXTPYT

SEQ ID NO: 73 - protein, 10 aa, synthetic construct
  REGION 1..10: note = Synthetic
  VARIANT 3: note = Xaa is Ser or Ala
  VARIANT 4: note = Xaa is Tyr or Asn
  VARIANT 5: note = Xaa is Ser or Thr
QQXXXTPRVT

SEQ ID NO: 74 - protein, 10 aa, synthetic construct
  REGION 1..10: note = Synthetic
  VARIANT 3: note = Xaa is Ser or Ala
  VARIANT 4: note = Xaa is Tyr or Asn
  VARIANT 5: note = Xaa is Ser or Thr
QQXXXFPRVS

SEQ ID NO: 75 - protein, 9 aa, synthetic construct
  REGION 1..9: note = Synthetic
  VARIANT 3: note = Xaa is Ser or Ala
  VARIANT 4: note = Xaa is Tyr or Asn
  VARIANT 5: note = Xaa is Ser or Thr
QQXXXWPRT

SEQ ID NO: 76 - protein, 9 aa, synthetic construct
  REGION 1..9: note = Synthetic
  VARIANT 9: note = Xaa is Tyr or Ser
SSDVGGYNX

SEQ ID NO: 77 - skipped

SEQ ID NO: 78 - protein, 17 aa, synthetic construct
  REGION 1..17: note = Synthetic
WISAYNGNTNYAQKLQG

SEQ ID NO: 79 - protein, 433 aa, synthetic construct
  REGION 1..433: note = Synthetic
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTAAATSITAYKSEGESAEFSFPLNLGEESLQGELRWKAEKAPSSQSWITFSLKNQKVSVQKSTSNPKFQLSETLPLTLQIPQVSLQFAGSGNLTLTLDRGILYQEVNLVVMKVTQPDSNTLTCEVMGPTSPKMRLILKQENQEARVSRQEKVIQVQAPEAGVWQCLLSEGEEVKMDSKIQVLSKGLNGSHHHHHH

SEQ ID NO: 80 - protein, 477 aa, synthetic construct
  REGION 1..477: note = Synthetic
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSHHHHHH

SEQ ID NO: 81 - protein, 432 aa, synthetic construct
  REGION 1..432: note = Synthetic
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTAAATSITAYKSEGESAEFSFPLNLGEESLQGELRWKAEKAPSSQSWITFSLKNQKVSVQKSTSNPKFQLSETLPLTLQIPQVSLQFAGSGNLTLTLDRGILYQEVNLVVMKVTQPDSNTLTCEVMGPTSPKMRLILKQENQEARVSRQEKVIQVQAPEAGVWQCLLSEGEEVKMDSKIQVLSKGLNGSHHHHHH

SEQ ID NO: 82 - protein, 476 aa, synthetic construct
  REGION 1..476: note = Synthetic
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTAAADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSHHHHHH

SEQ ID NO: 83 - protein, 272 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 84 - protein, 272 aa, Mus musculus [sequence omitted]

SEQ ID NO: 85 - protein, 457 aa, synthetic construct
  REGION 1..457: note = Synthetic
EVQLVQSGAEVKRPGASVKVSCKASGYPFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 86 - protein, 214 aa, synthetic construct; identical to SEQ ID NO: 48
  REGION 1..214: note = Synthetic

SEQ ID NO: 87 - protein, 457 aa, synthetic construct
  REGION 1..457: note = Synthetic
EVQLVQSGAEVKRPGASVKVSCKASGYTFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 88 - protein, 214 aa, synthetic construct; identical to SEQ ID NO: 50
  REGION 1..214: note = Synthetic

SEQ ID NO: 89 - protein, 457 aa, synthetic construct; identical to SEQ ID NO: 87
  REGION 1..457: note = Synthetic

SEQ ID NO: 90 - protein, 215 aa, synthetic construct; identical to SEQ ID NO: 52
  REGION 1..215: note = Synthetic

SEQ ID NO: 91 - protein, 457 aa, synthetic construct; identical to SEQ ID NO: 87
  REGION 1..457: note = Synthetic

SEQ ID NO: 92 - protein, 217 aa, synthetic construct; identical to SEQ ID NO: 62
  REGION 1..217: note = Synthetic

SEQ ID NO: 93 - protein, 452 aa, synthetic construct
  REGION 1..452: note = Synthetic
EVQLVQSGAEVKRPGASVKVSCKASGYTFTSYGISWVRQAPGQGLEWMGWISAYNGNTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 8 aa, synthetic construct
  REGION 1..8: note = Synthetic
GYTFTSYG

SEQ ID NO: 95 - protein, 8 aa, synthetic construct
  REGION 1..8: note = Synthetic
ISAYNGNT

SEQ ID NO: 96 - protein, 9 aa, synthetic construct
  REGION 1..9: note = Synthetic
SSDVGGYNS

SEQ ID NO: 97 - protein, 452 aa, synthetic construct
  REGION 1..452: note = Synthetic
EVQLVQSGAEVKRPGASVKVSCKASGYTFTSYGISWVRQAPGQGLEWMGWISAYNSNTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 8 aa, synthetic construct
  REGION 1..8: note = Synthetic G1/887_04_G12 and G1/894_08_A05
ISAYNSNT

SEQ ID NO: 99 - protein, 452 aa, synthetic construct; identical to SEQ ID NO: 97
  REGION 1..452: note = Synthetic

SEQ ID NO: 100 - protein, 6 aa, synthetic construct
  REGION 1..6: note = Synthetic
QSISGR

SEQ ID NO: 101 - protein, 452 aa, synthetic construct
  REGION 1..452: note = Synthetic
EVQLVQSGAEVKRPGASVKVSCKASGYPFTSYGISWVRQAPGQGLEWMGWISAYNSNTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 8 aa, synthetic construct
  REGION 1..8: note = Synthetic
GYPFTSYG

SEQ ID NO: 103 - protein, 6 aa, synthetic construct
  REGION 1..6: note = Synthetic
QSIGNR

SEQ ID NO: 104 - protein, 452 aa, synthetic construct; identical to SEQ ID NO: 97
  REGION 1..452: note = Synthetic

SEQ ID NO: 105 - protein, 452 aa, synthetic construct; identical to SEQ ID NO: 97
  REGION 1..452: note = Synthetic

SEQ ID NO: 106 - protein, 4 aa, synthetic construct
  REGION 1..4: note = Synthetic
EASN

SEQ ID NO: 107 - protein, 452 aa, synthetic construct
  REGION 1..452: note = Synthetic
EVQLVQSGAEVKRPGASVKVSCKASGYTFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - DNA, 1356 bp, synthetic construct
  misc_feature 1..1356: note = Synthetic
  source 1..1356: mol_type = other DNA
gaagtgcagctggtgcagtccggagccgaagtcaagaggcctggagcgtccgtgaaggtgtcctgcaaagcctcaggatacaccttcacttcgtacgggatttcctgggtccgccaagcaccgggtcaaggcttggagtggatgggatggatcagcgcgtattccgggggaaccaactacgctcaaaagctgcagggtcgcgtgaccatgaccaccgatacctccacctcaacggcctacatggaactgagatctctgcggagcgacgacactgccgtgtactactgtgcccgggacctgttccccactatcttcggagtgtcgtactactactactggggccaggggactctcgtgaccgtgtcgagcgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 109 - DNA, 1356 bp, synthetic construct
  misc_feature 1..1356: note = Synthetic
  source 1..1356: mol_type = other DNA
gaagtgcagctggtgcagtccggagccgaagtcaagaggcctggagcgtccgtgaaggtgtcctgcaaagcctcaggatacaccttcacttcgtacgggatttcctgggtccgccaagcaccgggtcaaggcttggagtggatgggatggatcagcgcgtattccgggggaaccaactacgctcaaaagctgcagggtcgcgtgaccatgaccaccgatacctccacctcaacggcctacatggaactgagatctctgcggagcgacgacactgccgtgtactactgtgcccgggacctgttccccactatcttcggagtgtcgtactactactactggggccaggggactctcgtgaccgtgtcgagcgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 110 - DNA, 369 bp, synthetic construct
  misc_feature 1..369: note = Synthetic
  source 1..369: mol_type = other DNA
gaagtgcagctggtgcagtccggagccgaagtcaagaggcctggagcgtccgtgaaggtgtcctgcaaagcctcaggatacaccttcacttcgtacgggatttcctgggtccgccaagcaccgggtcaaggcttggagtggatgggatggatcagcgcgtattccgggggaaccaactacgctcaaaagctgcagggtcgcgtgaccatgaccaccgatacctccacctcaacggcctacatggaactgagatctctgcggagcgacgacactgccgtgtactactgtgcccgggacctgttccccactatcttcggagtgtcgtactactactactggggccaggggactctcgtgaccgtgtcgagc

SEQ ID NO: 111 - protein, 8 aa, synthetic construct
  REGION 1..8: note = Synthetic
ISAYSGGT

SEQ ID NO: 112 - DNA, 645 bp, synthetic construct
  misc_feature 1..645: note = Synthetic
  source 1..645: mol_type = other DNA
gacattcagatgacccaatccccgtccacgctgagcgcctccgtcggtgatcgcgtgacaatcacttgtcgggcgtcgcagtccatctctggaaggctcgcctggtaccagcagaagcctggaaaggctcccaacctccttatctacgaagccagcaacctggagtccggagtgcctagccggttcagcggatcagggtccggtaccgagttcaccctgaccatttcctcgctccaacctgaggacttcgccacctactactgccaacagtcctattcaactccgcgcgtgaccttcggccagggcactaaggtcgaaatcaaaagaaccgtggcagccccatcggtgtttatcttcccgccctcggacgaacagctgaagtcaggcactgctagcgtggtctgtctcctgaacaatttctacccgcgcgaagctaaggtccagtggaaggtcgacaacgcgctgcagtccggaaacagccaggagtcagtgaccgagcaggactccaaggattccacttattccctgtcctccaccctgactttgagcaaggccgactacgagaagcacaaagtgtacgcctgcgaagtgacccatcaagggctttcgtcgcccgtgaccaagagcttcaaccggggcgaatgc

SEQ ID NO: 113 - DNA, 324 bp, synthetic construct
  misc_feature 1..324: note = Synthetic
  source 1..324: mol_type = other DNA
gacattcagatgacccaatccccgtccacgctgagcgcctccgtcggtgatcgcgtgacaatcacttgtcgggcgtcgcagtccatctctggaaggctcgcctggtaccagcagaagcctggaaaggctcccaacctccttatctacgaagccagcaacctggagtccggagtgcctagccggttcagcggatcagggtccggtaccgagttcaccctgaccatttcctcgctccaacctgaggacttcgccacctactactgccaacagtcctattcaactccgcgcgtgaccttcggccagggcactaaggtcgaaatcaaa

SEQ ID NO: 114 - protein, 452 aa, synthetic construct
  REGION 1..452: note = Synthetic
EVQLVQSGAEVKRPGASVKVSCKASGYPFTSYGISWVRQAPGQGLEWMGWISAYSGGTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - DNA, 1356 bp, synthetic construct
  misc_feature 1..1356: note = Synthetic
  source 1..1356: mol_type = other DNA
gaagtgcagctggtgcagtccggagccgaagtcaagaggcctggagcgtccgtgaaggtgtcctgcaaagcctcaggataccccttcacttcgtacgggatttcctgggtccgccaagcaccgggtcaaggcttggagtggatgggatggatcagcgcgtattccgggggaaccaactacgctcaaaagctgcagggtcgcgtgaccatgaccaccgatacctccacctcaacggcctacatggaactgagatctctgcggagcgacgacactgccgtgtactactgtgcccgggacctgttccccactatcttcggagtgtcgtactactactactggggccaggggactctcgtgaccgtgtcgagcgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaactgctgggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 116 - DNA, 1356 bp, synthetic construct
  misc_feature 1..1356: note = Synthetic
  source 1..1356: mol_type = other DNA
gaagtgcagctggtgcagtccggagccgaagtcaagaggcctggagcgtccgtgaaggtgtcctgcaaagcctcaggataccccttcacttcgtacgggatttcctgggtccgccaagcaccgggtcaaggcttggagtggatgggatggatcagcgcgtattccgggggaaccaactacgctcaaaagctgcagggtcgcgtgaccatgaccaccgatacctccacctcaacggcctacatggaactgagatctctgcggagcgacgacactgccgtgtactactgtgcccgggacctgttccccactatcttcggagtgtcgtactactactactggggccaggggactctcgtgaccgtgtcgagcgctagcactaagggcccgtcggtgttcccgctggccccatcgtccaagagcacatcagggggtaccgccgccctgggctgccttgtgaaggattactttcccgagcccgtcacagtgtcctggaacagcggagccctgacctccggagtgcatactttcccggctgtgcttcagtcctctggcctgtactcattgtcctccgtggtcaccgtcccttcgtcctccctgggcacccagacctatatctgtaatgtcaaccataagccctcgaacaccaaggtcgacaagaaggtcgagccgaagtcgtgcgacaagactcacacttgcccgccttgcccagccccggaagctgccggtggtccttcggtgttcctcttcccgcccaagccgaaggataccctgatgatctcacggacccccgaagtgacctgtgtggtggtggacgtgtcccacgaggacccggaagtgaaattcaattggtacgtggatggagtggaagtgcacaacgccaagaccaagccacgggaagaacagtacaactctacctaccgcgtggtgtccgtgctcactgtgctgcaccaagactggctgaacgggaaggagtacaagtgcaaagtgtccaacaaggcgctgcctgccccaattgagaaaactatctcgaaagccaagggacagcctcgagagcctcaagtgtacaccctgcctccctctcgggacgagctgaccaagaaccaagtctccctgacctgtctggtcaagggattctacccatcggatatcgccgtggaatgggaaagcaacggacagcccgagaacaactacaagacgactccgcccgtgctggattccgacgggagcttcttcttgtactccaagctgaccgtcgacaagagcagatggcagcagggaaacgtgttctcctgctccgtgatgcatgaggcgctgcacaaccactacactcagaagagcttgtccctgtcgcccgga

SEQ ID NO: 117 - DNA, 369 bp, synthetic construct
  misc_feature 1..369: note = Synthetic
  source 1..369: mol_type = other DNA
gaagtgcagctggtgcagtccggagccgaagtcaagaggcctggagcgtccgtgaaggtgtcctgcaaagcctcaggataccccttcacttcgtacgggatttcctgggtccgccaagcaccgggtcaaggcttggagtggatgggatggatcagcgcgtattccgggggaaccaactacgctcaaaagctgcagggtcgcgtgaccatgaccaccgatacctccacctcaacggcctacatggaactgagatctctgcggagcgacgacactgccgtgtactactgtgcccgggacctgttccccactatcttcggagtgtcgtactactactactggggccaggggactctcgtgaccgtgtcgagc

SEQ ID NO: 118 - DNA, 642 bp, synthetic construct
  misc_feature 1..642: note = Synthetic
  source 1..642: mol_type = other DNA
gacatccagatgacgcagagcccgtctaccctgtccgcctccgtgagagatcgcgtgatcatcacctgtcgggccagccagtccatcggaaaccgcttggcgtggtaccagcacaagcctgggaaggctccgaagctgctcatctacgaagcctcgacttcggagactggtgtccctagccggttcagcggatcgggatcagggaccgatttcactctgaccatttcctccctgcaacccgaggacttcgccacctactactgccaacagtcatattccaccccgtacaccttcggacaaggcaccaagctcgaaatcaagcggactgtcgccgcaccttccgtgttcattttcccaccctccgacgaacagctgaaatcgggtacagctagcgtggtctgtctcctgaacaatttctacccgcgcgaagctaaggtccagtggaaggtcgacaacgcgctgcagtccggaaacagccaggagtcagtgaccgagcaggactccaaggattccacttattccctgtcctccaccctgactttgagcaaggccgactacgagaagcacaaagtgtacgcctgcgaagtgacccatcaagggctttcgtcgcccgtgaccaagagcttcaaccggggcgaatgc

SEQ ID NO: 119 - DNA, 321 bp, synthetic construct
  misc_feature 1..321: note = Synthetic
  source 1..321: mol_type = other DNA
gacatccagatgacgcagagcccgtctaccctgtccgcctccgtgagagatcgcgtgatcatcacctgtcgggccagccagtccatcggaaaccgcttggcgtggtaccagcacaagcctgggaaggctccgaagctgctcatctacgaagcctcgacttcggagactggtgtccctagccggttcagcggatcgggatcagggaccgatttcactctgaccatttcctccctgcaacccgaggacttcgccacctactactgccaacagtcatattccaccccgtacaccttcggacaaggcaccaagctcgaaatcaag

SEQ ID NO: 120 - protein, 452 aa, synthetic construct; identical to SEQ ID NO: 107
  REGION 1..452: note = Synthetic

SEQ ID NO: 121 - DNA, 1356 bp, synthetic construct; identical to SEQ ID NO: 108
  misc_feature 1..1356: note = Synthetic
  source 1..1356: mol_type = other DNA

SEQ ID NO: 122 - DNA, 1356 bp, synthetic construct; identical to SEQ ID NO: 109
  misc_feature 1..1356: note = Synthetic
  source 1..1356: mol_type = other DNA

SEQ ID NO: 123 - DNA, 369 bp, synthetic construct; identical to SEQ ID NO: 110
  misc_feature 1..369: note = Synthetic
  source 1..369: mol_type = other DNA

SEQ ID NO: 124 - DNA, 642 bp, synthetic construct
  misc_feature 1..642: note = Synthetic
  source 1..642: mol_type = other DNA
gacattcagatgacccagtccccgagcacgctgtccgcaagcgtgggggacagagtgaccatcacttgccgcgcctcacaatccatcagcggacgcttggcctggtaccagcagaagcccggaaaggccccaaaccttctgatctacgaagcctcgaacctggagtcaggcgtcccttcgcggttctctggctccggttccggaactgagttcaccctcaccatctcgtccctgcaaccggaagatttcgccacctactactgccaacagtcgtactcctggccccggacattcggacagggaaccaaagtcgagattaagcggactgtggcggctcctagcgtgttcatctttcccccgtccgacgaacagctgaagtccggtaccgctagcgtggtctgtctcctgaacaatttctacccgcgcgaagctaaggtccagtggaaggtcgacaacgcgctgcagtccggaaacagccaggagtcagtgaccgagcaggactccaaggattccacttattccctgtcctccaccctgactttgagcaaggccgactacgagaagcacaaagtgtacgcctgcgaagtgacccatcaagggctttcgtcgcccgtgaccaagagcttcaaccggggcgaatgc

SEQ ID NO: 125 - DNA, 321 bp, synthetic construct
  misc_feature 1..321: note = Synthetic
  source 1..321: mol_type = other DNA
gacattcagatgacccagtccccgagcacgctgtccgcaagcgtgggggacagagtgaccatcacttgccgcgcctcacaatccatcagcggacgcttggcctggtaccagcagaagcccggaaaggccccaaaccttctgatctacgaagcctcgaacctggagtcaggcgtcccttcgcggttctctggctccggttccggaactgagttcaccctcaccatctcgtccctgcaaccggaagatttcgccacctactactgccaacagtcgtactcctggccccggacattcggacagggaaccaaagtcgagattaag

SEQ ID NO: 126 - protein, 452 aa, synthetic construct; identical to SEQ ID NO: 107
  REGION 1..452: note = Synthetic

SEQ ID NO: 127 - DNA, 1356 bp, synthetic construct; identical to SEQ ID NO: 108
  misc_feature 1..1356: note = Synthetic
  source 1..1356: mol_type = other DNA

SEQ ID NO: 128 - DNA, 1356 bp, synthetic construct; identical to SEQ ID NO: 109
  misc_feature 1..1356: note = Synthetic
  source 1..1356: mol_type = other DNA

SEQ ID NO: 129 - DNA, 369 bp, synthetic construct; identical to SEQ ID NO: 110
  misc_feature 1..369: note = Synthetic
  source 1..369: mol_type = other DNA

SEQ ID NO: 130 - DNA, 651 bp, synthetic construct
  misc_feature 1..651: note = Synthetic
  source 1..651: mol_type = other DNA
cagtcggcccttactcaacccgcgtcagtctccggtagccccggacagtccatcacgatttcgtgcaccggaaccagcagcgatgtcgggggatacaactacgtgtcctggtaccagcagttcccgggaaaggcccctaagctgatgatcttcgaagtcactaacagaccttccggagtgtcggaccggttctccggctccaagtccgacaacactgcgagcctgaccatctcgggcctgcaagccgaggacgaagccgagtactactgtagctcattcaagcgcggttccaccctcgtggtgttcggcggtggcactaagctcaccgtgctgggacagccagccgcagctcctagcgtgaccttgttccccccgtcgagcgaagaactgcaggccaacaaggccaccctcgtctgcctgatctccgacttctaccctggggccgtgactgtggcttggaaggccgattcaagcccagtgaaagccggagtggaaaccaccactccgtccaagcagtcgaacaataagtatgccgcgtcctcctacctgtcgctgaccccggagcagtggaagtcccatcggtcctactcctgccaagtcacccacgaagggtccactgtggagaaaacagtggctcccaccgagtgctct

SEQ ID NO: 131 - DNA, 336 bp, synthetic construct
  misc_feature 1..336: note = Synthetic
  source 1..336: mol_type = other DNA
cagtcggcccttactcaacccgcgtcagtctccggtagccccggacagtccatcacgatttcgtgcaccggaaccagcagcgatgtcgggggatacaactacgtgtcctggtaccagcagttcccgggaaaggcccctaagctgatgatcttcgaagtcactaacagaccttccggagtgtcggaccggttctccggctccaagtccgacaacactgcgagcctgaccatctcgggcctgcaagccgaggacgaagccgagtactactgtagctcattcaagcgcggttccaccctcgtggtgttcggcggtggcactaagctcaccgtgctggga

SEQ ID NO: 132 - protein, 9 aa, synthetic construct
  REGION 1..9: note = Synthetic
SSDVGGYNY

SEQ ID NO: 133 - protein, 452 aa, synthetic construct
  REGION 1..452: note = Synthetic
EVQLVQSGAEVKRPGASVKVSCKASGYTFTSYGISWVRQAPGQGLEWMGWISAYGGSTNYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 123 aa, synthetic construct
  REGION 1..123: note = Synthetic
EVQLVQSGAEVKRPGASVKVSCKASGYTFTSYGISWVRQAPGQGLEWMGWISAYGGSTNYAQKLQGRVTMTTDTSTSTAYMELRSLRSDDTAVYYCARDLFPTIFGVSYYYYWGQGTLVTVSS

SEQ ID NO: 135 - protein, 8 aa, synthetic construct
  REGION 1..8: note = Synthetic
ISAYGGST

SEQ ID NO: 136 - protein, 17 aa, synthetic construct
  REGION 1..17: note = Synthetic
WISAYGGSTNYAQKLQG

SEQ ID NO: 137 - protein, 216 aa, synthetic construct; identical to SEQ ID NO: 58
  REGION 1..216: note = Synthetic

SEQ ID NO: 138 - protein, 108 aa, synthetic construct; identical to SEQ ID NO: 32
  REGION 1..108: note = Synthetic

SEQ ID NO: 139 - protein, 452 aa, synthetic construct; identical to SEQ ID NO: 107
  REGION 1..452: note = Synthetic

SEQ ID NO: 140 - protein, 123 aa, synthetic construct; identical to SEQ ID NO: 29
  REGION 1..123: note = Synthetic

SEQ ID NO: 141 - protein, 216 aa, synthetic construct; identical to SEQ ID NO: 58
  REGION 1..216: note = Synthetic

SEQ ID NO: 142 - protein, 108 aa, synthetic construct; identical to SEQ ID NO: 32
  REGION 1..108: note = Synthetic

SEQ ID NO: 143 - protein, 452 aa, synthetic construct
  REGION 1..452: note = Synthetic
EVQLVQSGAEVKRPGASVKVSCKASGYTFTSYGISWVRQAPGQGLEWMGWISAYSGNANYAQKLQGRVTMTTDTSTSTAYMELRSLRSDDTAVYYCARDLFPTIFGVSYYYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 123 aa, synthetic construct
  REGION 1..123: note = Synthetic
EVQLVQSGAEVKRPGASVKVSCKASGYTFTSYGISWVRQAPGQGLEWMGWISAYSGNANYAQKLQGRVTMTTDTSTSTAYMELRSLRSDDTAVYYCARDLFPTIFGVSYYYYWGQGTLVTVSS

SEQ ID NO: 145 - protein, 8 aa, synthetic construct
  REGION 1..8: note = Synthetic
ISAYSGNA

SEQ ID NO: 146 - protein, 17 aa, synthetic construct
  REGION 1..17: note = Synthetic
WISAYSGNANYAQKLQG

SEQ ID NO: 147 - protein, 216 aa, synthetic construct; identical to SEQ ID NO: 58
  REGION 1..216: note = Synthetic

SEQ ID NO: 148 - protein, 108 aa, synthetic construct; identical to SEQ ID NO: 32
  REGION 1..108: note = Synthetic

SEQ ID NO: 149 - protein, 452 aa, synthetic construct; identical to SEQ ID NO: 133
  REGION 1..452: note = Synthetic

SEQ ID NO: 150 - protein, 123 aa, synthetic construct
  REGION 1..123: note = Synthetic
EVQLVQSGAEVKRPGASVKVSCKASGYPFTSYGISWVRQAPGQGLEWMGWISAYGGSTNYAQKLQGRVTMTTDTSTSTAYMELRSLRSDDTAVYYCARDLFPTIFGVSYYYYWGQGTLVTVSS

SEQ ID NO: 151 - protein, 215 aa, synthetic construct; identical to SEQ ID NO: 54
  REGION 1..215: note = Synthetic

SEQ ID NO: 152 - protein, 107 aa, synthetic construct; identical to SEQ ID NO: 28
  REGION 1..107: note = Synthetic

SEQ ID NO: 153 - protein, 452 aa, synthetic construct; identical to SEQ ID NO: 107
  REGION 1..452: note = Synthetic

SEQ ID NO: 154 - protein, 123 aa, synthetic construct; identical to SEQ ID NO: 27
  REGION 1..123: note = Synthetic

SEQ ID NO: 155 - protein, 215 aa, synthetic construct; identical to SEQ ID NO: 54
  REGION 1..215: note = Synthetic

SEQ ID NO: 156 - protein, 107 aa, synthetic construct; identical to SEQ ID NO: 28
  REGION 1..107: note = Synthetic

SEQ ID NO: 157 - protein, 452 aa, synthetic construct; identical to SEQ ID NO: 143
  REGION 1..452: note = Synthetic

SEQ ID NO: 158 - protein, 123 aa, synthetic construct
  REGION 1..123: note = Synthetic
EVQLVQSGAEVKRPGASVKVSCKASGYPFTSYGISWVRQAPGQGLEWMGWISAYSGNANYAQKLQGRVTMTTDTSTSTAYMELRSLRSDDTAVYYCARDLFPTIFGVSYYYYWGQGTLVTVSS

SEQ ID NO: 159 - protein, 215 aa, synthetic construct; identical to SEQ ID NO: 54
  REGION 1..215: note = Synthetic

SEQ ID NO: 160 - protein, 107 aa, synthetic construct; identical to SEQ ID NO: 28
  REGION 1..107: note = Synthetic

SEQ ID NO: 161 - protein, 452 aa, synthetic construct; identical to SEQ ID NO: 133
  REGION 1..452: note = Synthetic

SEQ ID NO: 162 - protein, 123 aa, synthetic construct; identical to SEQ ID NO: 134
  REGION 1..123: note = Synthetic

SEQ ID NO: 163 - protein, 215 aa, synthetic construct; identical to SEQ ID NO: 56
  REGION 1..215: note = Synthetic

SEQ ID NO: 164 - protein, 107 aa, synthetic construct; identical to SEQ ID NO: 30
  REGION 1..107: note = Synthetic

SEQ ID NO: 165 - protein, 452 aa, synthetic construct; identical to SEQ ID NO: 107
  REGION 1..452: note = Synthetic

SEQ ID NO: 166 - protein, 215 aa, synthetic construct; identical to SEQ ID NO: 56
  REGION 1..215: note = Synthetic

SEQ ID NO: 167 - protein, 107 aa, synthetic construct; identical to SEQ ID NO: 30
  REGION 1..107: note = Synthetic

SEQ ID NO: 168 - protein, 452 aa, synthetic construct; identical to SEQ ID NO: 143
  REGION 1..452: note = Synthetic

SEQ ID NO: 169 - protein, 123 aa, synthetic construct; identical to SEQ ID NO: 144
  REGION 1..123: note = Synthetic

SEQ ID NO: 170 - protein, 215 aa, synthetic construct; identical to SEQ ID NO: 56
  REGION 1..215: note = Synthetic

SEQ ID NO: 171 - protein, 107 aa, synthetic construct; identical to SEQ ID NO: 30
  REGION 1..107: note = Synthetic

SEQ ID NO: 172 - protein, 452 aa, synthetic construct; identical to SEQ ID NO: 133
  REGION 1..452: note = Synthetic

SEQ ID NO: 173 - protein, 123 aa, synthetic construct; identical to SEQ ID NO: 134
  REGION 1..123: note = Synthetic

SEQ ID NO: 174 - protein, 216 aa, synthetic construct; identical to SEQ ID NO: 60
  REGION 1..216: note = Synthetic

SEQ ID NO: 175 - protein, 108 aa, synthetic construct; identical to SEQ ID NO: 40
  REGION 1..108: note = Synthetic

SEQ ID NO: 176 - protein, 452 aa, synthetic construct; identical to SEQ ID NO: 107
  REGION 1..452: note = Synthetic

SEQ ID NO: 177 - protein, 123 aa, synthetic construct; identical to SEQ ID NO: 29
  REGION 1..123: note = Synthetic

SEQ ID NO: 178 - protein, 216 aa, synthetic construct; identical to SEQ ID NO: 60
  REGION 1..216: note = Synthetic

SEQ ID NO: 179 - protein, 108 aa, synthetic construct; identical to SEQ ID NO: 40
  REGION 1..108: note = Synthetic

SEQ ID NO: 180 - protein, 452 aa, synthetic construct; identical to SEQ ID NO: 143
  REGION 1..452: note = Synthetic

SEQ ID NO: 181 - protein, 123 aa, synthetic construct; identical to SEQ ID NO: 144
  REGION 1..123: note = Synthetic

SEQ ID NO: 182 - protein, 216 aa, synthetic construct; identical to SEQ ID NO: 60
  REGION 1..216: note = Synthetic

SEQ ID NO: 183 - protein, 108 aa, synthetic construct; identical to SEQ ID NO: 40
  REGION 1..108: note = Synthetic

SEQ ID NO: 184 - protein, 433 aa, synthetic construct
  REGION 1..433: note = Synthetic
MRIFAVFIFTIYWHLLNAFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERTAAATSITAYKSEGESAEFSFPLNLGEESLQGELRWKAEKAPSSQSWITFSLKNQKVSVQKSTSNPKFQLSETLPLTLQIPQVSLQFAGSGNLTLTLDRGILYQEVNLVVMKVTQPDSNTLTCEVMGPTSPKMRLILKQENQEARVSRQEKVIQVQAPEAGVWQCLLSEGEEVKMDSKIQVLSKGLNGSHHHHHH

(19) United States
(12) Patent Application Publication
(10) Pub. No.: US 2025/0270324 A1
(43) Pub. Date: Aug. 28, 2025

(54) ANTIBODY MOLECULES

(71) Applicant: INVOX PHARMA LIMITED, London (GB)

(72) Inventors: Francisca Wollerton, Cambridge (GB); Matthew Lakins, London (GB); Mateusz Wydro, Cambridge (GB); Sachin Surade, Cambridge (GB); Michael Dyson, Sawston Cambridgeshire (GB)

(73) Assignee: INVOX PHARMA LIMITED, London (GB)

(21) Appl. No.: 19/042,049

(22) Filed: Jan. 31, 2025

Related U.S. Application Data
(63) Continuation of application No. 17/259,642, filed on Jan. 12, 2021, now Pat. No. 12,247,074.

(30) Foreign Application Priority Data
Jul. 12, 2018 (GB) 1811403.3

(51) Int. Cl. C07K 16/28; A61K 39/00

(52) U.S. Cl. CPC C07K 16/2827; A61K 2039/505; C07K 2317/31; C07K 2317/526; C07K 2317/565; C07K 2317/71

(57) ABSTRACT

An antibody molecule, or antigen-binding fragment thereof capable of binding specifically to PD-L1.

### FIELD OF THE INVENTION

The invention relates to antibodies and antigen-binding fragments thereof that bind to programmed-death ligand 1 (PD-L1). The antibodies or antigen-binding fragments thereof comprise a CDR-based antigen-binding site for PD-L1. Antibodies or antigen-binding fragments thereof of the invention may find application, for example, in cancer therapy.

### BACKGROUND TO THE INVENTION

Programmed cell death 1 (PD-1) is a cell-surface receptor, its ligands PD-L1 (CD274, B7-H1) and PD-L2 (B7-DC) deliver inhibitory signals that regulate the balance between T cell activation, tolerance, and immunopathology. PD-L1 is transiently expressed on all immune cells and some tumour cells.

PD-L1 is a type I transmembrane protein with two Ig-like domains within the extracellular region, a transmembrane domain and a short cytoplasmic domain. The complete human PD-L1 (hPD-L1) sequence can be found under GENBANK® Accession No. Q9NZQ7. The cytoplasmic domain has no known signal transduction motif suggesting that there is no signalling by PD-L1 on interaction of the ligand with its receptor. The molecular weight of PD-L1 is 40 kDa (290 amino acids), it is encoded by the CD274 gene on human chromosome 9 and on mouse chromosome 19. PD-L1 is a member of the B7 protein family and shares approximately 20% amino acid sequence identity with B7.1 and B7.2. Human PD-L1 shares 70% and 93% amino acid identity with the murine and cynomolgus orthologs of PD-L1, respectively.

Human PD-L1 binds to its receptor. PD-1, with an affinity (KD) of 770 nM. PD-1 is expressed on activated T cells, B cells, and myeloid cells; it modulates activation or inhibition of cellular immune responses. Binding of PD-L1 to PD-1 delivers an inhibitory signal, reducing cytokine production and suppressing proliferation of T cells. Consequently, PD-L1 expression by cells can mediate protection against cytotoxic T lymphocyte (CTL) killing and is a regulatory mechanism that dampens chronic immune responses during viral infections. Cancer, as a chronic and pro-inflammatory disease, subverts this immune-protective pathway through up-regulation of PD-L1 expression to evade the host immune response. In the context of an active immune response. IFNγ also upregulates the expression of PD-L1. PD-L1 also mediates immune suppression through interaction with another protein, B7.1 (also known as CD80), blocking its ability to deliver one of the secondary signals of activation on T cells through CD28. In terms of PD-L1 expression on tumour cells and its engagement with B7.1, the relevance of this specific interaction in tumour immune resistance is still unclear.

PD-L1 expression has been shown in a wide variety of solid tumours. Of 654 samples examined in one study, spanning 19 tumours from different sites, 89 (14%) were PD-L1 positive (≥5% frequency). The highest PD-L1 positive frequencies were seen in head and neck (17/54; 31%), cervical (10/34; 29%), cancer of unknown primary origin (CUP; 8/29; 28%), glioblastoma multiforme (GBM; 5/20; 25%), bladder (8/37: 21%), oesophageal (16/80; 20%), triple negative (TN) breast (6/33; 18%), and hepatocarcinoma (6/41; 15%) (Grosso et al., 2013). Tumour-associated expression of PD-L1 has been shown to confer immune resistance and potentially protect tumour cells from T cell mediated apoptosis.

Therapies targeting PD-L1 have shown excellent results in murine in vivo studies. In the B16 murine model of melanoma, treatment with anti-PD-L1 therapy combined with either GVAX or FVAX vaccination strategies resulted in a significant effect both on survival (30 days for control vs 52 days for PD-L1-treated) and percentage of tumour-free (5%) animals upon conclusion of the study (Curran et al., 2010). Anti-PD-L1 therapy has also been used to study the mechanism of immune suppression in the P815 murine mastoma model. P815 cells injected into mice normally trigger a strong immune response, which results in their rejection. When PD-L1 is expressed on P815 cells, these cells escape immune attack, which in turn can be negated through administration of anti-PD-L1 antibodies (Iwai et al., 2002). It is evident that targeting the PD-1/PD-L1 axis in immunogenic human cancers (Herbst et al, 2014) results in a survival advantage through stimulation of an anti-cancer immune response (Wolchok et al., 2013; Larkin et al., 2015).

Atezolizumab (MPDL3280A, RG7466, TECENTRIQ™) is a humanized IgG1 antibody which binds to PD-L1. It is in clinical trials as a monotherapy and also in combination with other biologic and/or small molecule therapies for treatment of solid cancers, including colorectal cancer, breast cancer, non-small-cell lung carcinoma, bladder cancer, and renal cell carcinoma. Treatment with atezolizumab resulted in objective response rates (ORR) of 23% in NSCLC, 36% melanoma, 33% bladder, 14% in RCC, and 13% in head and neck cancers (Herbst et al., 2014; Powles et al., 2014).

In May 2016 the FDA granted accelerated approval to atezolizumab for locally advanced or metastatic urothelial carcinoma treatment after failure of cisplatin-based chemotherapy; however, the confirmatory trial failed to achieve its primary endpoint of overall survival. In October 2016, the FDA approved atezolizumab for the treatment of patients with metastatic non-small cell lung cancer (NSCLC) who had disease progression during or following platinum-containing chemotherapy. Patients with EGFR or ALK genomic tumor aberrations are required to have disease progression on FDA-approved therapy for these aberrations prior to receiving atezolizumab. Atezolizumab in combination with avastin and standard chemotherapy for some patients with lung cancer is under FDA priority review, a decision being expected by 5 Sep. 2018. The most common adverse effects reported in clinical studies of atezolizumab were fatigue, decreased appetite, nausea, and infections; urinary tract infection was the most common severe adverse effect.

Avelumab (MSB0010718C, BAVENCIO™) is a fully human IgG1 antibody which binds to PD-L1 and is undergoing clinical testing in a number of cancers including bladder cancer, gastric cancer, head and neck cancer, mesothelioma, non-small-cell lung carcinoma, ovarian cancer, renal cancer and Merkel-cell carcinoma. Avelumab received orphan drug designation by the European Medicines Agency (EMA) for the treatment of gastric cancer in January 2017. In 2017 the FDA and the EMA approved avelumab for Merkel-cell carcinoma (an aggressive skin cancer) in adults and pediatric patients 12 years and older. Approval was based on data from an open-label, single-arm, multi-center clinical trial (JAVELIN Merkel 200 trial). All patients had histologically-confirmed metastatic MCC with disease progression on or after chemotherapy administered for metastatic disease. The overall response rate (ORR) was assessed by an independent review committee according to Response Evaluation Criteria in Solid Tumors (RECIST) 1.1. The ORR was 33% (95% confidence interval [CI]: 23.3, 43.8), with 11% complete and 22% partial response rates. Among the 29 responding patients, the response duration ranged from 2.8 to 23.3+ months with 86% of responses durable for 6 months or longer. Responses were observed in patients regardless of PD-L1 tumor expression or presence of Merkel cell polyomavirus. Safety data were evaluated in 1738 patients. The most common serious adverse reactions to avelumab were immune-mediated adverse reactions (pneumonitis, hepatitis, colitis, adrenal insufficiency, hypo- and hyperthyroidism, diabetes mellitus, and nephritis) and life-threatening infusion reactions. Among the 88 patients enrolled in the JAVELIN Merkel 200 trial, the most common adverse reactions were fatigue, musculoskeletal pain, diarrhea, nausea, infusion-related reaction, rash, decreased appetite, and peripheral edema. Serious adverse reactions that occurred in more than one patient in the trial were acute kidney injury, anemia, abdominal pain, ileus, asthenia, and cellulitis.

Durvalumab (MEDI4736, IMFINZI™) is a human IgG1 antibody which binds to PD-L1 and is being tested in clinical trials alone or in combination with tremelimumab in non-small-cell lung cancer. squamous cell carcinoma of the head and neck, bladder cancer, pancreatic cancer and with other biologic and small molecules in trials for additional solid cancers such as gastric cancers, melanoma and unresectable hepatocellular carcinoma.

Durvalumab was approved by the FDA for the treatment of patients with locally advanced or metastatic urothelial carcinoma who either have disease progression during or following platinum-containing chemotherapy or have disease progression within 12 months of neoadjuvant or adjuvant treatment with platinum-containing chemotherapy.

A phase 1B clinical trial of durvalumab and tremelimumab showed some activity in non-small cell lung cancer (NSCLC). However, in July 2017, AstraZeneca announced that a phase III trial of durvalumab with tremelimumab as a first-line treatment of non-small cell lung cancer had failed to meet its primary endpoint of progression-free survival.

Early results of a phase I trial combining durvalumab and gefitinib in lung cancer patients were reported to have “showed promise”. A phase 1 clinical trial is in progress using durvalumab with a TLR 7/8 agonist (MEDI 9197) for solid tumors. A Phase 1b/2a trial is in progress combining durvalumab with an HPV DNA vaccine (MEDI 0457) in patients with HPV-associated recurrent/metastatic head and neck cancer.

In November 2017, the double-blinded phase 3 AstraZeneca PACIFIC clinical trial reported the efficacy of durvalumab in the treatment of stage III non-small cell lung cancer. A cohort of 709 patients with stage III NSCLC, who did not have disease progression after two or more cycles of a platinum-based chemotherapy, were randomly assigned to receive durvalumab or a placebo as consolidation therapy for their lung cancer. Durvalumab increased the median progression-free survival from 5.6 months (placebo) to 16.8 months (durvalumab); the 12 month progression-free survival rate was increased from 35.3% (placebo) to 55.9% (durvalumab), and the 18 month progression-free survival rate was increased from 27.0% (placebo) to 44.2% (durvalumab). The median time to death or distant metastases increased from 14.6 months (placebo) to 23.2 months (durvalumab). However, extreme side effects were also increased from 26.1% of patients (placebo) to 29.9% of patients (durvalumab).

Adverse effects were reported following exposure to durvalumab in 182 patients with locally advanced or metastatic urothelial carcinoma whose disease has progressed during or after one standard platinum-based regimen. Patients received 10 mg/kg durvalumab via intravenous infusion every 2 weeks. The median duration of exposure was 10.2 weeks (range: 0.14, 52.4). Thirty-one percent (31%) of patients had a drug delay or interruption for an adverse reaction. The most common (>2%) were liver injury (4.9%), urinary tract infection (3.3%), acute kidney injury (3.3%), and musculoskeletal pain (2.7%). The most common adverse reactions (≥15%) were fatigue (39%), musculoskeletal pain (24%), constipation (21%), decreased appetite (19%), nausea (16%), peripheral edema (15%) and urinary tract infection (15%). The most common Grade 3 or 4 adverse reactions (≥3%) were fatigue, urinary tract infection, musculoskeletal pain, abdominal pain, dehydration, and general physical health deterioration. Eight patients (4.4%) who were treated with durvalumab experienced Grade 5 adverse events of cardiorespiratory arrest, general physical health deterioration, sepsis, ileus, pneumonitis, or immune-mediated hepatitis. Three additional patients were experiencing infection and disease progression at the time of death. Durvalumab was discontinued for adverse reactions in 3.3% of patients. Serious adverse reactions occurred in 46% of patients. The most frequent serious adverse reactions (>2%) were acute kidney injury (4.9%), urinary tract infection (4.4%), musculoskeletal pain (4.4%), liver injury (3.3%), general physical health deterioration (3.3%), sepsis, abdominal pain, pyrexia/tumor associated fever (2.7% each). Immune-mediated adverse reactions requiring systemic corticosteroids or hormone replacement therapy occurred in 8.2% (15/182) patients, including 5.5% (10/182) patients who required systemic corticosteroid therapy and 2.7% (5/182) patients who required only hormone replacement therapy. Seven patients (3.8%) received an oral prednisone dose equivalent to >40 mg daily for an immune-mediated adverse reaction.

Further anti-PD-L1 antibodies including BMS-936559 have been tested in clinical trials, and others are in preclinical testing.

WO2013181634 (Sorrento Therapeutics) describes PD-L1 antibodies. Only one antibody disclosed, “SH1E2” (SEQ ID NO: 147/148 in that application), is said to exhibit improved T-cell activation, measured by the percentage of CD25 positive cells, when compared to PD-L1 antibodies 10A5 and YW234.55S70 disclosed in the art.

Infectious diseases show many parallels with oncology. It is thought that the role of PD-L1 in immune regulation could be harnessed to maximize the immune response against pathogens. Immunomodulation in infectious disease is an emerging area of medicine and early reviews suggest that PD-L1 blockade may improve biological responses to infection, in particular, by helping to counteract T cell exhaustion, manage immune-mediated clearance and generate long-term immunity (Wykes and Lewin, 2017). Thus, there also remains a need in the art for additional molecules which can target PD-L1 and which find application in the treatment of infectious diseases.

Antibodies that target PD-L1 may also be useful to treat conditions associated with inflammation, such as vascular inflammation and stroke.

Whilst there are various anti-PD-L1 therapeutics in development, current data shows that overall treatment with existing anti-PD-L1 monotherapies results in a response in less than 50% of cancer patients. The spectrum and severity of reported adverse reactions differs between antibodies in clinical testing. To increase the objective response rate (ORR), and/or seek to reduce adverse effects, anti-PD-L1 antibodies may be combined with other biologics, such as antibodies against other checkpoint regulators, as well as with small molecule therapies and other immune system activating approaches, such as tumour vaccines.

Thus, there remains a need in the art for additional molecules which can target PD-L1 and which find application in cancer therapy.

### STATEMENTS OF INVENTION

The present inventors have prepared anti-PD-L1 antibodies by screening a phage library, followed by mutagenesis, screening, selection, and light chain shuffling to isolate anti-PD-L1 antibodies with affinity for PD-L1 and activity in a T cell activation assay.

Further rounds of mutagenesis, screening and selection were performed to remove potential sites for post-translational modifications and to improve the biophysical properties of the selected antibodies.

The above approach enabled identification of anti-PD-L1 antibodies which showed excellent binding to PD-L1 and activity in T cell activation assays. Based on these characteristics, it is expected that the antibodies of the invention will find application in the treatment of human cancers, as well as infectious and inflammatory diseases, through inhibition of PD-L1.

Antibodies of the invention were also shown to have a high affinity for cynomolgus PD-L1, comparable to their affinity for human PD-L1. Antibodies of the invention also showed measurable affinity for mouse PD-L1.

In addition, antibodies to PD-L1 were identified that had a relatively high melting temperature, which can be expected to have enhanced stability, beneficial in the manufacture and storage of the antibodies.

The invention provides:
1. An antibody or antigen-binding fragment thereof, capable of binding specifically to PD-L1, comprising a variable heavy (VH) domain comprising heavy chain complementarity determining regions (CDRs): HCDR1, HCRD2 and HCDR3, characterised in that the amino acid sequence of HCDR1 (amino acids 31 to 35) is SYGIS (SEQ ID NO: 1); the amino acid sequence of HCDR2 is WISAYX₁X₂X₃X₄NYAQKLQG (SEQ ID NO: 2); and the amino acid sequence of HCDR3 is DLFPTIFGVSYYYY (SEQ ID NO: 3); wherein X₁ is S or N or G; X₂ is G or S; X₃ is or G, N or S; and X₄ is T or A, and wherein the sequences are defined by Kabat nomenclature.
2. An antibody or antigen-binding fragment thereof according to clause 1, characterised in that the amino acid sequence of HCDR1 (amino acids 31 to 35) is SYGIS (SEQ ID NO: 1); the amino acid sequence of HCDR2 is WISAYX₁X₂X₃X₄NYAQKLQG (SEQ ID NO: 2); and the amino acid sequence of HCDR3 is DLFPTIFGVSYYYY (SEQ ID NO: 3); wherein X₁ is S or N; X₂ is G or S; X₃ is G or N; and X₄ is T, and wherein the sequences are defined by Kabat nomenclature.
3. An antibody or antigen-binding fragment thereof according to clause 1 or clause 2, wherein the amino acid at position 28 preceding HCDR1 is P or T.
4. An antibody or antigen-binding fragment thereof according to any preceding clause, wherein the sequence X₁X₂X₃X₄ (SEQ ID NO: 4) (residues 54-57) of HCDR2 is selected from SGGT (SEQ ID NO: 5), NSNT (SEQ ID NO: 6), GGST (SEQ ID NO: 7) and SGNA (SEQ ID NO: 8).
5. An antibody or antigen-binding fragment thereof according to any preceding clause, wherein the residue at HCDR1 position 28 (Kabat nomenclature) is P and the sequence X₁X₂X₃X₄ (SEQ ID NO: 4 (residues 54-57) of HCDR2 is SGGT (SEQ ID NO: 5).
6. An antibody or antigen-binding fragment thereof according to any preceding clause, comprising a variable light (VL) domain comprising light chain complementarity determining regions: LCDR1, LCDR2 and LCDR3, characterised in that:
(a) the VL is a kappa VL and the amino acid sequence of LCDR1 is RASQSIX₅X₆RLA (SEQ ID NO: 9); the amino acid sequence of LCDR2 is EASX₇X₈EX₉ (SEQ ID NO: 10); and the amino acid sequence of LCDR3 is QQX₁₀X₁₁X₁₂X₁₃PX₁₄X₁₅X₁₆ (SEQ ID NO: 11); wherein X₅ is G or S; X₆ is N or G; X₇ is T or N; X₈ is S or L; X₉ is T or S; X₁₀ is S or A; X₁₁ is Y or N; X₁₂ is S or T; X₁₃ is T, W or F; X₁₄ is absent or R; X₁₅ is Y, R or V; and X₁₆ is T or S; or,
(b) the VL is a lambda VL and the amino acid sequence of LCDR1 is TGTSSDVGGYNX₁₇VS (SEQ ID NO: 12); the amino acid sequence of LCDR2 is EVTNRPS (SEQ ID NO: 13); and the amino acid sequence of LCDR3 is SSFKRGSTLVV (SEQ ID NO: 14); wherein X₁₇ is Y or S; and wherein the sequences are defined by Kabat nomenclature.
7. An antibody or antigen-binding fragment thereof according to clause 6, wherein the VL domain is a kappa VL and the amino acid sequence of LCDR1 is RASQSIGNRLA (SEQ ID NO: 15), the amino acid sequence of LCDR2 is EASTSET (SEQ ID NO: 16), and the amino acid sequence of LCDR3 is QQSYSTPYT (SEQ ID NO: 17).
8. An antibody or antigen-binding fragment thereof according to any preceding clause comprising an antigen-binding site comprising the CDRs (HCDR1, HCRD2, HCDR3, LCDR1, LCDR2 and LCDR3, respectively) of antibody:
(a) G1AA/E12v2 of SEQ ID NO: 1, 18, 3, 15, 16 and 17;
(b) G1AA/G12v2 of SEQ ID NO: 1, 18, 3, 19, 20 and 21;
(c) G1AA/E05v2 of SEQ ID NO: 1, 18, 3, 19, 20 and 22;
(d) G1/887_04_E12 of SEQ ID NO: 1, 23, 3, 15, 16 and 17;
(e) G1/887_04_G12 of SEQ ID NO: 1, 23, 3, 19, 20 and 21;
(f) G1/894_08_E05 of SEQ ID NO: 1, 23, 3, 19, 20 and 22;
(g) G1/894_08_A05 of SEQ ID NO: 1, 23, 3, 19, 20 and 24;
(h) G1AA/lambdav3 of SEQ ID NO: 1, 18, 3, 25, 13 and 14;
(i) G1/280_02_G02_NS of SEQ ID NO: 1, 23, 3, 26, 13 and 14 or
(j) G1/280_02_G02 of SEQ ID NO: 1, 78, 3, 26, 13 and 14;
wherein the sequences are defined according to Kabat nomenclature.
9. An antibody or antigen-binding fragment according to any preceding clause, wherein the antigen-binding site comprises the VH and/or VL domain of antibody:
(a) G1AA/E12v2 of SEQ ID NO: 27 and 28, respectively;
(b) G1AA/G12v2 of SEQ ID NO: 29 and 30, respectively;
(c) G1AA/E05v2 of SEQ ID NO: 31 and 32, respectively;
(d) G1/887_04_E12 of SEQ ID NO: 33 and 34, respectively;
(e) G1/887_04_G12 of SEQ ID NO: 35 and 36, respectively;
(f) G1/894_08_E05 of SEQ ID NO: 37 and 38, respectively;
(g) G1/894_08_A05 of SEQ ID NO: 39 and 40, respectively;
(h) G1AA/lambdav3 of SEQ ID NO: 41 and 42, respectively;
(i) G1/280_02_G02_NS of SEQ ID NO: 43 and 44, respectively; or
(j) G1/280_02_G02 of SEQ ID NO: 45 and 46, respectively;
wherein the sequences are defined according to the Kabat nomenclature.
10. The antibody molecule according to any preceding clause, wherein the antibody molecule comprises the heavy chain and/or light chain of antibody:
(a) G1AA/E12v2 of SEQ ID NO: 47 and 48, respectively;
(b) G1AA/G12v2 of SEQ ID NO: 49 and 50, respectively;
(c) G1AA/E05v2 of SEQ ID NO: 51 and 52, respectively;
(d) G1/887_04_E12 of SEQ ID NO: 53 and 54, respectively;
(e) G1/887_04_G12 of SEQ ID NO: 55 and 56, respectively;
(f) G1/894_08_E05 of SEQ ID NO: 57 and 58, respectively;
(g) G1/894_08_A05 of SEQ ID NO: 59 and 60, respectively;
(h) G1AA/lambdav3 of SEQ ID NO: 61 and 62, respectively;
(i) G1/280_02_G02_NS of SEQ ID NO: 63 and 64, respectively; or
(j) G1/280_02_G02 of SEQ ID NO: 65 and 66, respectively;
wherein the sequences are defined according to Kabat nomenclature.
11. An antibody or antigen-binding fragment thereof according to any preceding clause, comprising the HCDRs (HCDR1, HCDR2 and HCDR3) and/or LCDRs (LCDR1, LCDR2 and LCDR3); VH and/or VL; Fab; light chain and/or heavy chain of antibody G1AA/E12v2, G1AA/G12v2 or G1AA/E05v2.
12. The antibody or antigen-binding fragment thereof, according to any preceding clause, comprising the HCDRs (HCDR1, HCDR2 and HCDR3) and/or LCDRs (LCDR1, LCDR2 and LCDR3); VH and/or VL; Fab; light chain and/or heavy chain of antibody G1AA/E12v2 or G1/E12v2.
13. An antibody or antigen-binding fragment thereof according to any preceding clause, wherein the VH has at least 95, 96, 97, 98 or 99% identity to the VH of an antibody selected from G1AA/E12v2 of SEQ ID NO: 27, G1AA/G12v2 of SEQ ID NO: 29, G1AA/E05v2 of SEQ ID NO: 31, G1/887_04_E12 of SEQ ID NO: 33, G1/887_04_G12 of SEQ ID NO: 35. G1/894_08_E05 of SEQ ID NO: 37, G1/894_08_A05 of SEQ ID NO: 39 G1AA/lambdav3 of SEQ ID NO: 41,
G1/280_02_G02_NS of SEQ ID NO: 43 and G1/280_02_G02 of SEQ ID NO: 45.
14. An antibody or antigen-binding fragment thereof, according to any preceding clause, wherein the antibody molecule, or antigen-binding fragment, binds to human PD-L1.
15. An antibody or antigen-binding fragment thereof, according to any preceding clause, wherein the antibody molecule, or antigen-binding fragment, binds to cynomolgus PD-L1.
16. An antibody or antigen-binding fragment thereof, according to any preceding clause, wherein the antibody or antigen-binding fragment, binds to mouse PD-L1.
17. An antibody or antigen-binding fragment thereof, according to any preceding clause, wherein the antibody or antigen-binding fragment has an affinity (KD) for recombinant human PD-L1 and for recombinant cynomolgus PD-L1 of less than 2 nM, preferably less than 1 nM, more preferably less than 0.75 nM, yet more preferably less than 0.5 nM when measured by SPR (e.g., Biacore).
18. An antibody or antigen-binding fragment thereof, according to any preceding clause, wherein the antibody or antigen-binding fragment thereof enhances T-cell activation when assessed in a Mixed Lymphocyte Reaction (MLR) assay.
19. An antibody or antigen-binding fragment thereof, according to any preceding clause, wherein the antibody or antigen-binding fragment, is a multispecific, preferably a bispecific, molecule comprising at least a second antigen-binding site.
20. An antibody or antigen-binding fragment, according to any preceding clause, wherein the antibody or antigen-binding fragment thereof, comprises a second antigen-binding site located in a constant domain of the antibody or antigen-binding fragment.
21. An antibody or antigen-binding fragment thereof, according to clause 20, wherein the second antigen-binding site comprises:
(a) a first sequence in the AB structural loop and/or a second sequence in the EF structural loop of a constant heavy domain,
(b) a first sequence in the AB structural loop and a second sequence in the EF structural loop of a constant heavy domain,
(c) a first sequence in the AB structural loop and/or a second sequence in the EF structural loop and/or a third sequence in the CD structural loop of a constant heavy domain
(d) a first sequence in the AB structural loop, a second sequence in the EF structural loop and a third sequence in the CD structural loop of a constant heavy domain
22. An antibody or antigen-binding fragment thereof, according to clause 20 or 21, wherein the constant heavy domain is a CH3 domain.
23. An antibody or antigen-binding fragment thereof, according to any preceding clause, wherein the antibody is an immunoglobulin G (IgG), or antigen-binding fragment thereof.
24. An antibody or antigen-binding fragment thereof, according to clause 23, wherein the antibody is an IgG1 or fragment thereof, or an IgG4 or fragment thereof.
25. An antibody or antigen-binding fragment thereof, according to clause 23 or clause 24, wherein the antibody is an IgG1 or fragment thereof with a modified Fc region.
26. An antibody or antigen-binding fragment thereof, according to clause 24 or clause 25, wherein the antibody is an IgG1 or fragment thereof with a modified Fc region with reduced immune effector function.
27. An antibody or antigen-binding fragment thereof, according to clause 25 or 26, wherein the modified Fc has reduced ADCC and/or CDC relative to IgG1.
28. An antibody or antigen-binding fragment thereof, according to any of clauses 25 to 27, wherein the modified Fc region comprises a LALA, LALA-PA or LALA-PG modification.
29. An antibody or antigen-binding fragment thereof, according to any of clauses 25 to 28, wherein the antibody is an IgG1 or antigen-binding fragment thereof comprising a LALA modification in the Fc region.
30. An antibody or antigen-binding fragment thereof, according to any of clauses 19 to 29, wherein the second antigen-binding site binds to an inhibitory checkpoint molecule, costimulatory molecule or tumour-associated antigen.

In an antibody or antigen-binding fragment thereof, according to the invention, the second antigen-binding site may bind to an inhibitory checkpoint molecule, such as CTLA-4, LAG-3, TIGIT, TIM-3, VISTA, CD73, CSF-1R, KIR. B7-H3, B7-H4, 2B4, NKG2A, CD47, SIRPa, BTLA, CCR4, CD200R, or TGFbeta.

In an antibody or antigen-binding fragment thereof, according to the invention, the second antigen-binding site may bind to and be an agonist for a costimulatory molecule expressed by T cells such as OX40, ICOS, CD40, HVEM, NKG2D, or TNFR2.

In an antibody or antigen-binding fragment thereof, according to the invention, the second antigen-binding site may bind to a tumour-associated antigen (TAA), such as c-Met, B7-H3, B7-H4, EGFR, HER-2, EPCAM, CEACAM, FAP, VEGF, MSLN, GPC3, CD38, CD19, or CD20.
31. An antibody or antigen-binding fragment thereof, according to any of clauses 19 to 30, wherein the second antigen-binding site does not bind to OX40, Inducible T-cell COStimulator (ICOS) or CD137.
32. An antibody or antigen-binding fragment thereof, according to any of clauses 19 to 30, wherein the second antigen-binding site does not bind to CD27 or glucocorticoid-induced TNFR-related protein (GITR).
33. An antibody or antigen-binding fragment thereof, according to any of clauses 19 to 30, wherein the second antigen-binding site does not bind to lymphocyte-activation gene 3 (LAG-3).
34. A conjugate or fusion comprising an antibody or antigen-binding fragment thereof according to any preceding clause and an immune system modulator (agonist or antagonist), a cytotoxic molecule, or a radioisotope.
35. An antibody, antigen-binding fragment thereof, conjugate or fusion according to any preceding clause having a detectable label.
36. A nucleic acid molecule or set of nucleic acid molecules encoding an antibody, antigen-binding fragment thereof, conjugate or fusion according to any preceding clause.
37. A nucleic acid molecule or set of nucleic acid molecules according to clause 36, wherein the nucleic acid molecule or set of nucleic acid molecules comprises cDNA sequence encoding one or more of the VH and/or VL, Fab, heavy and/or light chain of:
(a) G1AA/E12v2 or G1/E12v2;
(b) G1AA/E05v2 or G1/E05v2;
(c) G1AA/G12v2 or G1/G12v2;
(d) G1/887_04_E12;
(e) G1/894_08_E05;
(f) G1/887_04_G12;
(g) G1/894_08_A05;
(h) G1AA/lambdav3;
(i) G1/280_02_G02_NS; or
(j) G1/280_02_G02.
38. A nucleic acid molecule or set of nucleic acid molecules according to clause 37, comprising a first nucleic acid sequence and a second nucleic acid sequence, wherein:
(a) the first nucleic acid sequence comprises a VH cDNA sequence encoding the VH of antibody G1AA/E12v2 of SEQ ID NO: 27 and the second nucleic acid sequence comprises a VL cDNA sequence encoding the VL of antibody G1AA/E12v2 of SEQ ID NO: 28;
(b) the first nucleic acid sequence comprises a VH cDNA sequence encoding the VH of antibody G1AA/G12v2 of SEQ ID NO: 29 and the second nucleic acid sequence comprises a VL cDNA sequence encoding the VL of antibody G1AA/G12v2 of SEQ ID NO: 30;
(c) the first nucleic acid sequence comprises a VH cDNA sequence encoding the VH of antibody G1AA/E05v2 of SEQ ID NO: 31 and the second nucleic acid sequence comprises a VL cDNA sequence encoding the VL of antibody G1AA/E05v2 of SEQ ID NO: 32;
(d) the first nucleic acid sequence comprises a VH cDNA sequence encoding the VH of antibody G1/887_04_E12 of SEQ ID NO: 33 and the second nucleic acid sequence comprises the VL cDNA sequence of antibody G1/887_04_E12 of SEQ ID NO: 34;
(e) the first nucleic acid sequence comprises a VH cDNA sequence encoding the VH of antibody G1/887_04_G12 of SEQ ID NO: 35 and the second nucleic acid sequence comprises a VL cDNA sequence encoding the VL of antibody G1/887_04_G12 of SEQ ID NO: 36;
(f) the first nucleic acid sequence comprises a VH cDNA sequence encoding the VH of antibody G1/894_08_E05 of SEQ ID NO: 37 and the second nucleic acid sequence comprises a VL cDNA sequence encoding the VL of antibody G1/894_08_E05 of SEQ ID NO: 38;
(g) the first nucleic acid sequence comprises a VH cDNA sequence encoding the VH of antibody G1/894_08_A05 of SEQ ID NO: 39 and the second nucleic acid sequence comprises a VL cDNA sequence encoding the VL of antibody G1/894_08_A05 of SEQ ID NO: 40;
(h) the first nucleic acid sequence comprises a VH cDNA sequence encoding the VH of antibody G1AA/lambdav3 of SEQ ID NO: 41 and the second nucleic acid sequence comprises a VL cDNA sequence encoding the VL of antibody G1AA/lambdav3 of SEQ ID NO: 42;
(i) the first nucleic acid sequence comprises a VH cDNA sequence encoding the VH of antibody G1/280_02_G02_NS of SEQ ID NO: 43 and the second nucleic acid sequence comprises a VL cDNA sequence encoding the VL of antibody G1/280_02_G02_NS of SEQ ID NO: 44; or
(i) the first nucleic acid sequence comprises a VH cDNA sequence encoding the VH of antibody G1/280_02_G02 of SEQ ID NO: 45 and the second nucleic acid sequence comprises a VL cDNA sequence encoding the VL of antibody G1/280_02_G02 of SEQ ID NO: 46.
39. A vector or set of vectors comprising the nucleic acid molecule or set of nucleic acid molecules of any of clauses 36 to 38.
40. A recombinant host cell comprising a nucleic acid molecule or set of nucleic acid molecules of any of clauses 36 to 38, or the vector or set of vectors of clause 39.
41. A method of producing an antibody antigen-binding fragment thereof, conjugate or fusion according to any preceding clause, comprising culturing the recombinant host cell of clause 40 under conditions suitable for production of the antibody, antigen-binding fragment, conjugate or fusion.
42. The method of clause 41 further comprising isolating and/or purifying the antibody, antigen-binding fragment, conjugate or fusion.
43. A composition (e.g., pharmaceutical composition) comprising the antibody, antigen-binding fragment, conjugate or fusion according to any of clauses 1 to 42 and an excipient (e.g., pharmaceutically-acceptable excipient).
44. An antibody, antigen-binding fragment, conjugate or fusion according to any of clauses 1 to 35 or composition according to clause 43 for use in a method for treatment of the human or animal body by therapy.
45. A method for treatment of a disease or disorder in a patient comprising administering to the patient a therapeutically-effective amount of an antibody, antigen-binding fragment thereof, conjugate or fusion according to any of clauses 1 to 35 or composition according to clause 43.
46. The use of an antibody, antigen-binding fragment thereof, conjugate or fusion according to any of clauses 1 to 35 or composition according to clause 43 in the manufacture of a medicament for the treatment of the human or animal body.
47. An antibody, antigen-binding fragment thereof, conjugate or fusion according to any of clauses 1 to 35 or composition according to clause 43 for use according to clause 44 in a method of treatment that comprises administering the antibody, antigen-binding fragment thereof, conjugate, fusion or composition to the human or animal body in combination with a second therapeutic.
48. A method of clause 45, or use of clause 46, wherein the method further comprises administering a therapeutically-effective amount of a second therapeutic to the patient.
49. An antibody, antigen-binding fragment thereof, conjugate, fusion or composition for use according to clause 47, or in a method of clause 48, wherein the second therapeutic is a radiotherapy, preferably targeted radiotherapy.
50. An antibody, antigen-binding fragment, conjugate or fusion according to any of clauses 1 to 35 or composition according to clause 43 for use in a diagnostic method practised on the human or animal body or practised in vitro on a sample from on the human or animal body.
51. A method of detecting a disease or disorder in a patient, the method comprising the use of an antibody, antigen-binding fragment thereof, conjugate or fusion according to any of clauses 1 to 35 or of a composition according to clause 43.
52. The use of an antibody, antigen-binding fragment thereof, conjugate or fusion according to any of clauses 1 to 35 or of a composition according to clause 43 in the manufacture of a diagnostic product.

Treatment against various types of cancer using anti-PD-L1 or anti-PD-1 antibodies has been investigated in clinical trials and shown promising results. These include solid tumours such as ovarian cancer, prostate cancer, colorectal cancer, fibrosarcoma, renal cell carcinoma, melanoma (advanced and metastatic melanoma), pancreatic cancer, breast cancer, glioblastoma multiforme, lung cancer (such as non-small cell lung cancer and small cell lung cancer), head and neck cancer (such as head and neck squamous cell carcinoma), stomach cancer (gastric cancer), bladder cancer, cervical cancer, uterine cancer (uterine endometrial cancer, uterine cervical cancer), vulvar cancer, testicular cancer, penile cancer, esophageal cancer, hepatocellular carcinoma, nasopharyngeal cancer, Merkel cell carcinoma, mesothelioma, DNA mismatch repair deficient colorectal cancer, DNA mismatch repair deficient endometrial cancer, thyroid cancer, Hodgkin's lymphoma, non-Hodgkin's lymphoma (such as diffuse large B-cell lymphoma, follicular lymphoma, indolent non-Hodgkin's lymphoma, mantle cell lymphoma), leukaemia (such as chronic lymphocytic leukaemia, myeloid leukaemia, acute lymphoblastoid leukaemia, or chronic lymphoblastoid leukaemia), multiple myeloma, and peripheral T-cell lymphoma. The antibody or antigen-binding fragment thereof of the invention thus may find application in the treatment of these cancers. Tumours of these cancers are known, or expected, to contain immune cells, such as TILs, expressing PD-L1.

In particular, treatment of melanoma, colorectal cancer, breast cancer, bladder cancer, renal cell carcinoma, gastric cancer, head and neck cancer (such as squamous cell carcinoma of the head and neck), mesothelioma, lung cancer (such as non-small-cell lung cancer), ovarian cancer, Merkel-cell carcinoma, pancreatic cancer, melanoma and hepatocellular carcinoma using anti-PD-L1 antibodies has been investigated in clinical trials and shown promising results. Thus, the cancer to be treated using an antibody or antigen-binding fragment thereof of the invention may be a melanoma, colorectal cancer, breast cancer, bladder cancer, renal cell carcinoma, bladder cancer, gastric cancer, head and neck cancer (such as squamous cell carcinoma of the head and neck), mesothelioma, lung cancer (such as non-small-cell lung cancer), ovarian cancer, Merkel-cell carcinoma, pancreatic cancer, melanoma, or hepatocellular carcinoma.

Cancer may be characterised by the abnormal proliferation of malignant cancer cells. Where the application refers to a particular type of cancer, such as breast cancer, this refers to a malignant transformation of the relevant tissue, in this case a breast tissue. A cancer which originates from malignant transformation of a different tissue, e.g., ovarian tissue, may result in metastatic lesions in another location in the body, such as the breast, but is not thereby a breast cancer as referred to herein but an ovarian cancer.

The cancer may be a primary or secondary cancer. Thus, an antibody or antigen-binding fragment thereof of the invention may be for use in a method of treating cancer in a patient, wherein the cancer is a primary tumour and/or a tumour metastasis.

An antibody or antigen-binding fragment thereof of the invention may also be expected to find application in the treatment of infectious diseases, such as viral, bacterial, fungal and/or parasitic infections. Preferably, the infectious disease is a viral, bacterial or fungal disease, more preferably a viral or bacterial disease, most preferably a viral disease. The infectious disease may be chronic or acute, but is preferably chronic.

Examples of viral diseases which may be treated with an antibody or antigen-binding fragment thereof according to the invention include: human immunodeficiency virus (HIV), influenza virus, enterovirus, hepatitis B virus (HBV), hepatitis C virus (HCV), hepatitis A virus (HAV), hepatitis D virus (HDV), and hepatitis E virus (HEV), respiratory syncytial virus (RSV), herpesvirus (such as Epstein-Barr virus, herpes simplex virus 1 (HSV-1), herpes simplex virus 2 (HSV-2), cytomegalovirus (CMV)), and papillomavirus infection.

Examples of bacterial diseases which may be treated with an antibody or antigen-binding fragment thereof of the invention include: Mycobacterium tuberculosis, gram-negative bacteria (such as Acinetobacter, Klebisella, Enterobacter), gram-positive bacteria (such as Clostridium difficile, Staphylococcus aureus), and Listeria (e.g., Listeria monocytogenes) infection.

Examples of fungal diseases which may be treated with an antibody or antigen-binding fragment thereof of the invention include: Aspergillus and Candida infection.

Examples of parasitic diseases which may be treated with an antibody or antigen-binding fragment thereof of the invention include: Malaria, Toxoplasma, and Leishmania infection.

An antibody or antigen-binding fragment thereof according to the invention is designed to be used in methods of treatment of patients, preferably human patients. An antibody or antigen-binding fragment thereof of the invention will usually be administered in the form of a pharmaceutical composition, which may comprise at least one additional component, such as a pharmaceutically acceptable excipient. For example, a pharmaceutical composition of the invention, may comprise, in addition to the antibody or antigen-binding fragment thereof, a pharmaceutically-acceptable excipient, carrier, buffer, stabiliser or other materials well known to those skilled in the art. Such materials should be non-toxic and should not interfere with the efficacy of the antibody or antigen-binding fragment thereof. The precise nature of the carrier or other material will depend on the route of administration, which may be by injection, e.g., intravenous or subcutaneous. The antibody or antigen-binding fragment thereof may be administered intravenously, or subcutaneously.

Liquid pharmaceutical compositions generally comprise a liquid carrier such as water or physiological saline solution. For subcutaneous or intravenous injection, or injection at the site of affliction, the antibody or antigen-binding fragment thereof, or pharmaceutical composition comprising the antibody or antigen-binding fragment thereof, is preferably in the form of a parenterally acceptable aqueous solution which is pyrogen-free and has suitable pH, isotonicity and stability.

A composition comprising an antibody or antigen-binding fragment thereof according to the invention may be administered alone or in combination with other treatments, concurrently or sequentially or as a combined preparation with another therapeutic agent or agents, dependent upon the condition to be treated. For example, an antibody or fragment thereof of the invention may be administered in combination with an existing therapeutic agent for the disease to be treated, e.g., a cancer as mentioned above. For example, an antibody or fragment thereof of the invention may be administered to the patient in combination with a second anti-cancer therapy, such as chemotherapy, anti-tumour vaccination (also referred to as a cancer vaccination), radiotherapy, immunotherapy, an oncolytic virus, chimeric antigen receptor (CAR) T-cell therapy, or hormone therapy.

It is expected that the antibody or fragment thereof of the invention may act as an adjuvant in anti-cancer therapy, such as chemotherapy, anti-tumour vaccination, or radiotherapy. Without wishing to be bound by theory, it is thought that administration of the antibody or fragment thereof to the patient as part of chemotherapy, anti-tumour vaccination, or radiotherapy will trigger a greater immune response against the cancer associated antigen PD-L1, than is achieved with chemotherapy, anti-tumour vaccination, or radiotherapy alone.

A method of treating cancer in a patient may thus comprise administering to the patient a therapeutically effective amount of an antibody or fragment thereof according to the invention in combination with a chemotherapeutic agent, anti-tumour vaccine, radionuclide, immunotherapeutic agent, oncolytic virus, CAR-T cell, or agent for hormone therapy. The chemotherapeutic agent, anti-tumour vaccine, radionuclide, immunotherapeutic agent, oncolytic virus, CAR-T cell, or agent for hormone therapy is preferably a chemotherapeutic agent, anti-tumour vaccine, radionuclide, immunotherapeutic agent, oncolytic virus, CAR-T cell, or agent for hormone therapy for the cancer in question, i.e., a chemotherapeutic agent, anti-tumour vaccine, radionuclide, immunotherapeutic agent, oncolytic virus, CAR-T cell, or agent for hormone therapy which has been shown to be effective in the treatment of the cancer in question. The selection of a suitable chemotherapeutic agent, anti-tumour vaccine, radionuclide, immunotherapeutic agent, oncolytic virus, CAR-T cell, or agent for hormone therapy, which have been shown to be effective for the cancer in question, is well within the capabilities of the skilled practitioner.

For example, where the method comprises administering to the patient a therapeutically effective amount of an antibody or fragment thereof according to the invention in combination with a chemotherapeutic agent, the chemotherapeutic agent may be selected from the group consisting of: taxanes, cytotoxic antibiotics, tyrosine kinase inhibitors, PARP inhibitors, B_RAF enzyme inhibitors, alkylating agents, platinum analogues, nucleoside analogues, thalidomide derivatives, antineoplastic chemotherapeutic agents and others. Taxanes include docetaxel, paclitaxel and nab-paclitaxel; cytotoxic antibiotics include actinomycin, bleomycin, anthracyclines, doxorubicin and valrubicin; tyrosine kinase inhibitors include sunitinib, erlotinib, gefitinib, axitinib, PLX3397, imatinib, cobemitinib and trametinib; PARP inhibitors include piraparib; B-Raf enzyme inhibitors include vemurafenib and dabrafenib; alkylating agents include dacarbazine, cyclophosphamide, temozolomide; platinum analogues include carboplatin, cisplatin and oxaliplatin; nucleoside analogues include gemcitabine and azacitidine; antineoplastics include fludarabine. Other chemotherapeutic agents suitable for use in the invention include methotrexate, defactinib, entinostat, pemetrexed, capecitabine, eribulin, irinotecan, fluorouracil, and vinblastine.

Vaccination strategies for the treatment of cancers have been implemented in the clinic and discussed in detail within scientific literature (such as Rosenberg S. Development of Cancer Vaccines. ASCO Educational Book Spring: 60-62 (2000)). This mainly involves strategies to prompt the immune system to respond to various cellular markers expressed by autologous or allogenic cancer cells by using those cells as a vaccination method, both with or without granulocyte-macrophage colony-stimulating factor (GM-CSF). GM-CSF provokes a strong response in antigen presentation and works particularly well when employed with said strategies.

Where a method of the invention comprises administering to the patient a therapeutically-effective amount of an antibody or fragment thereof according to the invention in combination with an immunotherapeutic agent, the immunotherapeutic agent may be selected from the group consisting of: antibodies binding to a checkpoint inhibitor, costimulatory molecule or soluble factor, such as antibodies binding to CTLA-4, LAG-3, TIGIT, TIM-3, VISTA, CD73, CSF-1R, KIR, OX40, CD40. HEVM. TGFB, IL-10, CSF-1. Alternatively, the immunotherapeutic agent may one or more cytokines or cytokine-based therapies selected from the group consisting of IL-2, prodrug of conjugated IL2, GM-CSF, IL-7, IL-12, IL-9. IL-15, IL-18, IL-21, and type I interferon.

Administration may be in a “therapeutically effective amount”, this being an amount which is sufficient to show benefit to a patient. Such benefit may be at least amelioration of at least one symptom. Thus, “treatment” of a specified disease refers to amelioration of at least one symptom. The actual amount administered, and rate and time-course of administration, will depend on the nature and severity of what is being treated, the particular patient being treated, the clinical condition of the individual patient, the cause of the disorder, the site of delivery of the composition, the type of antibody or fragment thereof, the method of administration, the scheduling of administration and other factors known to medical practitioners. Prescription of treatment, e.g., decisions on dosage etc., is within the responsibility of general practitioners and other medical doctors, and may depend on the severity of the symptoms and/or progression of a disease being treated. Appropriate doses of antibody or fragment thereof are well known in the art (Ledermann et al. (1991) Int. J. Cancer 47:659-664; and Bagshawe et al. (1991) Antibody, Immunoconjugates and Radiopharmaceuticals 4:915-922). Specific dosages indicated herein, or in the Physician's Desk Reference (2003) as appropriate for an antibody or fragment thereof being administered, may be used. A therapeutically-effective amount or suitable dose of an antibody or fragment thereof can be determined by comparing its in vitro activity and in vivo activity in an animal model. Methods for extrapolation of effective dosages in mice and other test animals to humans are known. The precise dose will depend upon a number of factors, including the size and location of the area to be treated, and the precise nature of the specific binding member. Treatments may be repeated at daily, twice-weekly, weekly or monthly intervals, at the discretion of the physician. Treatment may be given before and/or after surgery, and may be administered or applied directly at the anatomical site of surgical treatment.

### DETAILED DESCRIPTION

The invention relates to antibodies and antigen-binding fragments thereof that comprise a CDR-based antigen-binding site for PD-L1. An antibody or antigen-binding fragment thereof of the invention may be produced by recombinant means. A “recombinant antibody” is an antibody which has been produced by a recombinantly engineered host cell. An antibody or antigen-binding fragment thereof in accordance with the invention is optionally isolated or purified.

The term “PD-L1” may refer to human PD-L1, murine, in particular mouse PD-L1, and/or cynomolgus monkey PD-L1, unless the context requires otherwise. Preferably the term “PD-L1” refers to human PD-L1, unless the context requires otherwise.

The term “antibody molecule” describes an immunoglobulin whether natural or partly or wholly synthetically produced. The antibody molecule may be human or humanised. The antibody molecule is preferably a monoclonal antibody molecule. Examples of antibodies are the immunoglobulin isotypes, such as immunoglobulin G, and their isotypic subclasses, such as IgG1, IgG2, IgG3 and IgG4, as well as fragments thereof. The four human subclasses (IgG1, IgG2, IgG3 and IgG4) each contain a different heavy chain; but they are highly homologous and differ mainly in the hinge region and the extent to which they activate the host immune system. IgG1 and IgG4 contain two inter-chain disulphide bonds in the hinge region, IgG2 has 4 and IgG3 has 11 inter-chain disulphide bonds.

The terms “antibody” and “antibody molecule”, as used herein, includes antibody fragments, such as Fab and scFv fragments, provided that said fragments comprise a CDR-based antigen binding site for PD-L1. Unless the context requires otherwise, the terms “antibody” or “antibody molecule”, as used herein, is thus equivalent to “antibody or antigen-binding fragment thereof”.

Antibodies are immunoglobulins, which have the same basic structure consisting of two heavy and two light chains forming two Fab arms containing identical domains that are attached by a flexible hinge region to the stem of the antibody, the Fc domain, giving the classical ‘Y’ shape. The Fab domains consist of two variable and two constant domains, with a variable heavy (VH) and constant heavy 1 (CH1) domain on the heavy chain and a variable light (VL) and constant light (CL) domain on the light chain. The two variable domains (VH and VL) form the variable fragment (Fv), which provides the CDR-based antigen specificity of the antibody, with the constant domains (CH1 and VL) acting as a structural framework. Each variable domain contains three hypervariable loops, known as complementarity determining regions (CDRs). On each of the VH and VL the three CDRs (CDR1, CDR2, and CDR3) are flanked by four less-variable framework (FR) regions (FR1, FW2, FW3 and FW4) to give a structure FW1-CDR1-FW2-CDR2-FW3-CDR3-FW4. The CDRs provide a specific antigen recognition site on the surface of the antibody.

Both Kabat and ImMunoGeneTics (IMGT) numbering nomenclature is used herein. Generally, unless otherwise indicated (explicitly or by context) amino acid residues are numbered herein according to the Kabat numbering scheme (Kabat et al., 1991). For those instances when IMGT numbering is used, amino acid residues are numbered herein according to the ImMunoGeneTics (IMGT) numbering scheme. The IMGT numbering scheme is described in Lefranc et al., 2005. When the sequences are defined by IMGT nomenclature, the invention provides:
1A. An antibody or antigen-binding fragment thereof, capable of binding specifically to PD-L1 comprising a variable heavy (VH) domain comprising heavy chain CDRs: HCDR1, HCRD2 and HCDR3, each flanked by framework (FW) regions, characterised in that the amino acid sequence of HCDR1 is GYX₁FTSYG (SEQ ID NO: 67); the amino acid sequence of HCDR2 is ISAYX₂X₃X₄X₅ (SEQ ID NO: 68); and the amino acid sequence of HCDR3 is ARDLFPTIFGVSYYYY (SEQ ID NO: 69); wherein X₁ is P or T; X₂ is S, N or G, preferably S or N; X₃ is G or S; X₄ is G, N or S, preferably G or N; and X₅ is T or A, preferably T, and wherein the sequences are defined by the ImMunoGeneTics (IMGT) nomenclature.
2A. An antibody or antigen-binding fragment thereof according to clause 1A, wherein the sequence X₂X₃X₄X₅ (SEQ ID NO: 4) (residues 62-65) of HCDR2 is selected from SGGT (SEQ ID NO: 5), NSNT (SEQ ID NO: 6), GGST (SEQ ID NO: 7) and SGNA (SEQ ID NO: 8). (IMGT nomenclature).
3A. An antibody or antigen-binding fragment thereof according to clause 1A or 2A, wherein X₁ is P and X₂X₃X₄X₅ of HCDR2 is SGGT (SEQ ID NO: 5) (IMGT nomenclature).
4A. An antibody or antigen-binding fragment thereof according to any of clauses 1A to 3A comprising a variable light (VL) domain comprising a LCDR1, LCDR2 and LCDR3, characterised in that:
(a) the VL is a kappa VL and the amino acid sequence of LCDR1 is QSIX₆X₇R (SEQ ID NO: 70): the amino acid sequence of LCDR2 is EAS (SEQ ID NO: 71); and the amino acid sequence of LCDR3 is QQX₈X₉X₁₀TPYT (SEQ ID NO: 72), QQX₈X₉X₁₀TPRVT (SEQ ID NO: 73), QQX₈X₉X₁₀FPRVS (SEQ ID NO: 74), or QQX₈X₉X₁₀WPRT (SEQ ID NO: 75); wherein X₆ is G or S; X₇ is N or G; X₈ is S or A; X₉ is Y or N; and X₁₀ is S or T; or,
(b) the VL is a lambda VL and the amino acid sequence of LCDR1 is SSDVGGYNX₁₁ (SEQ ID NO: 76), the amino acid sequence of LCDR2 is EVT (SEQ ID NO: 77) and the amino acid sequence of LCDR3 is SSFKRGSTLVV (SEQ ID NO: 14); wherein X₁₁ is Y or S; and wherein the sequences are defined by IMGT nomenclature.
5A. An antibody antigen-binding fragment thereof according to any of clauses 1A to 4A, comprising an antigen-binding site comprising:
(a) the CDRs of antibody G1AA/E12v2;
(b) the CDRs of antibody G1AA/G12v2;
(c) the CDRs of antibody G1AA/E05v2;
(d) the CDRs of antibody G1/887_04_E12;
(e) the CDRs of antibody G1/887_04_G12;
(f) the CDRs of antibody G1/894_08_E05;
(g) the CDRs of antibody G1/894_08_A05;
(h) the CDRs of antibody G1AA/lambdav3; or
(i) the CDRs of antibody G1/280_02_G02_NS;
(j) the CDRs of antibody G1/280_02_G02;
wherein the sequences are defined according to the ImMunoGeneTics (IMGT) numbering scheme

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant region. Introduction of the CDRs of one immunoglobulin into another immunoglobulin is described for example in EP-A-184187, GB2188638A or EP-A-239400. Alternatively, a hybridoma or other cell producing an antibody molecule may be subject to genetic mutation or other changes, which may or may not alter the binding specificity of antibodies produced.

As antibodies can be modified in a number of ways, the term “antibody” should be construed as covering antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

An example of an antibody fragment comprising both CDR sequences and CH3 domain is a minibody, which comprises a scFv joined to a CH3 domain (Hu et al., 1996).

An antibody or antigen-binding fragment of the invention binds to PD-L1, in particular human PD-L1. Binding in this context may refer to specific binding. The term “specific” may refer to the situation in which the antibody molecule will not show any significant binding to molecules other than its specific binding partner(s), here PD-L1. The term “specific” is also applicable where the antibody molecule is specific for particular epitopes, such as epitopes on PD-L1, that are carried by a number of antigens in which case the antibody molecule will be able to bind to the various antigens carrying the epitope.

Amino acids may be referred to by their one letter or three letter codes, or by their full name. The one and three letter codes, as well as the full names, of each of the twenty standard amino acids are set out below.

| Amino acid | One letter code | Three letter code |
|---|---|---|
| alanine | A | Ala |
| arginine | R | Arg |
| asparagine | N | Asn |
| aspartic acid | D | Asp |
| cysteine | C | Cys |
| glutamic acid | E | Glu |
| glutamine | Q | Gln |
| glycine | G | Gly |
| histidine | H | His |
| isoleucine | I | Ile |
| leucine | L | Leu |
| lysine | K | Lys |
| methionine | M | Met |
| phenylalanine | F | Phe |
| proline | P | Pro |
| serine | S | Ser |
| threonine | T | Thr |
| tryptophan | W | Trp |
| tyrosine | Y | Tyr |
| valine | V | Val |

### Amino Acids, One and Three-Letter Codes

In preferred embodiments, the PD-L1 antibody of the invention comprises the HCDR3 sequence of E12v2 (SEQ ID NO: 3); it is preferred that the antibody further comprises the HCDR2 sequence of E12v2 (SEQ ID NO: 18); it is preferred that the PD-L1 antibody of the invention yet further comprises the HCDR1 sequence of E12v2 (SEQ ID NO: 1). In preferred embodiments the HCDR2 sequence is HCDR2 sequence of E12v2 (SEQ ID NO: 18) and the amino acid at position 28 in the VH (Kabat) is proline. In particularly preferred embodiments the PD-L1 antibody of the invention comprises the HCDR3 sequence of SEQ ID NO: 3, the HCDR2 sequence of SEQ ID NO: 18 and the amino acid at position 28 in the VH (Kabat) is proline. In more particularly preferred embodiments the PD-L1 antibody of the invention comprises the HCDR3 sequence of SEQ ID NO: 3, the HCDR2 sequence of SEQ ID NO: 18, and the HCDR1 sequence of SEQ ID NO: 1 and the amino acid at position 28 in the VH (Kabat) is proline.

Antibodies of the invention may comprise one or more, e.g., 1, 2, 3, 4, 5, 6, 7, 8, 9 or 10 further amino acid modifications in the VH and/or VL sequences, provided that functional properties of the antibody are retained.

A modification may be an amino acid substitution, deletion or insertion. Preferably, the modification is a substitution.

In preferred embodiments in which one or more amino acids are substituted with another amino acid, the substitutions may conservative substitutions, for example according to the following chart. In some embodiments, amino acids in the same category in the middle column are substituted for one another, i.e. a non-polar amino acid is substituted with another non-polar amino acid, for example. In some embodiments, amino acids in the same line in the rightmost column are substituted for one another.

| | | |
|---|---|---|
| ALIPHATIC | Non-polar | G A P |
| | | I L V |
| | Polar- | C S T M |
| | uncharged | N Q |
| | Polar-charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g., binding affinity) of the antibody molecule comprising the substitution as compared to the equivalent unsubstituted antibody molecule.

In a preferred embodiment, a PD-L1 antibody of the invention may comprise a VH and/or VL domain sequence with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the VH and/or VL sequences of the invention set forth herein.

In a preferred embodiment, an antibody of the invention comprises the HCDR3 domain of E12v2 set forth in SEQ ID NO: 3.

In another preferred embodiment, an antibody of the invention comprises the VH domain of E12v2 set forth in SEQ ID NO: 27 or a VH domain with an amino acid sequence which has at least 95%, at least 96%, at least 97%, at least 98%, or at least 99% sequence identity to the sequence set forth in SEQ ID NO: 27.

In a preferred embodiment, an antibody of the invention comprises a VH domain comprising the HCDR3 set forth in SEQ ID NO: 3 and the VH domain has an amino acid sequence with at least 70%, at least 75%, at least 80%, at least 85%, at least 90%, at least 95%, at least 96%, at least 97%, at least 98%, or at least 99% sequence identity to the sequence set forth in SEQ ID NO: 27.

In a preferred embodiment, an antibody of the invention comprises a VH domain comprising the HCDR3 of E12v2 set forth in SEQ ID NO: 3 and a HCDR2 selected from those set forth in SEQ ID NO: 18, 23 or 24 and the VH domain has an amino acid sequence with at least 70%, at least 75%, at least 80%, at least 85%, at least 90%, at least 95%, at least 96%, at least 97%, at least 98%, or at least 99% sequence identity to the sequence set forth in SEQ ID NO: 27.

In a preferred embodiment, an antibody of the invention comprises a VH domain comprising the HCDR3 of E12v2 set forth in SEQ ID NO: 3, a HCDR2 domain selected from those set forth in SEQ ID NO: 18, 23 or 24, a proline at position 28, and the VH domain has an amino acid sequence with at least 70%, at least 75%, at least 80%, at least 85%, at least 90%, at least 95%, at least 96%, at least 97%, at least 98%, or at least 99% sequence identity to the sequence set forth in SEQ ID NO: 27.

In a preferred embodiment, an antibody of the invention comprises a VL domain comprising a VL domain of E12v2 set forth in SEQ ID NO: 28 or an amino acid sequence with at least 70%, at least 75%, at least 80%, at least 85%, at least 90%, at least 95%, at least 96%, at least 97%, at least 98%, or at least 99% sequence identity to the sequence set forth in SEQ ID NO: 28.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used. e.g., BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm (Nucl. Acids Res. (1997) 25 3389-3402) may be used. Sequence identity may be defined using the Bioedit, ClustalW algorithm.

The antibody may comprise a CH2 domain. The CH2 domain is preferably located at the N-terminus of the CH3 domain, as in the case in a human IgG molecule. The CH2 domain of the antibody is preferably the CH2 domain of human IgG1, IgG2, IgG3, or IgG4, more preferably the CH2 domain of human IgG1. The sequences of human IgG domains are known in the art.

The antibody may comprise an immunoglobulin hinge region, or part thereof, at the N-terminus of the CH2 domain. The immunoglobulin hinge region allows the two CH2-CH3 domain sequences to associate and form a dimer. Preferably, the hinge region, or part thereof, is a human IgG1, IgG2, IgG3 or IgG4 hinge region, or part thereof. More preferably, the hinge region, or part thereof, is an IgG1 hinge region, or part thereof.

The sequence of the CH3 domain, is not particularly limited. Preferably, the CH3 domain is a human immunoglobulin G domain, such as a human IgG1, IgG2, IgG3, or IgG4 CH3 domain, most preferably a human IgG1 CH3 domain.

An antibody of the invention may comprise a human IgG1, IgG2, IgG3, or IgG4 constant region. The sequences of human IgG1, IgG2, IgG3, or IgG4 CH3 domains are known in the art.

The heavy chain of the antibody molecule may optionally comprise an additional lysine residue (K) at the C-terminus of the heavy chain CH3 domain sequence.

Immunoglobulins are known to have a modular architecture comprising discrete domains, which can be combined in a multitude of different ways to create multispecific, e.g., bispecific, trispecific, or tetraspecific antibody formats. Exemplary multispecific antibody formats are described in Spiess et al., 2015 and Kontermann, 2012, for example. The antibodies of the invention may be employed in such multispecific formats.

For example, an antibody of the invention may be a heterodimeric antibody molecule, such as a heterodimeric complete immunoglobulin molecule, or a fragment thereof. In this case, one part of the antibody will have a sequence or sequences as described herein. For example, where the antibody of the invention is a bispecific heterodimeric antibody molecule, the antibody may comprise a heavy chain and light chain as described herein paired with a heavy chain and light chain comprising a VH domain and a VL domain, respectively, which bind an antigen other than PD-L1. Techniques for preparing heterodimeric antibodies are known in the art and include knobs-into-holes (KIHs) technology, which involves engineering the CH3 domains of an antibody molecule to create either a “knob” or a “hole” to promote chain heterodimerization. Alternatively, heterodimeric antibodies can be prepared through the introduction of charge pairs into the antibody molecule to avoid homodimerization of CH3 domains by electrostatic repulsion and to direct heterodimerization by electrostatic attraction. Examples of heterodimeric antibody formats include CrossMab, mAb-Fv, SEED-body, and KIH IgG.

Alternatively, a multispecific antibody molecule may comprise a complete immunoglobulin molecule or a fragment thereof and an additional antigen-binding moiety or moieties. The antigen-binding moiety may for example be an Fv, scFv or single domain antibody, and may be fused to the complete immunoglobulin molecule or a fragment thereof. Examples of multispecific antibody molecules comprising additional antigen-binding moieties fused to a complete immunoglobulin molecule include DVD-IgG, DVI-IgG, scFv4-IgG, IgG-scFv, and scFv-IgG molecules (Spiess et al., 2015; FIG. 1). Examples of multispecific antibody molecules comprising additional antigen-binding moieties fused to an immunoglobulin fragment include BiTE molecules, diabodies, and DART molecules, for example (Spiess et al., 2015; FIG. 1). Other suitable formats would be readily apparent to the skilled person.

In addition to the CDR-based PD-L1-binding site, e.g., in the VH of an antibody, the antibody may further comprise one or more additional antigen-binding sites to create a bi- or multi-specific molecule. The antibody may comprise a CH3-based or CH2-based antigen-binding site. CDR-based antigen binding sites are found in naturally-occurring immunoglobulin molecules and their structure is well-known in the art. Where the antibody or antigen-binding fragment thereof comprises a CDR-based antigen binding site, the antibody or antigen-binding fragment thereof is preferably an antibody molecule. The bi- or multispecific antibody molecule may comprise a CDR-based antigen binding site for PD-L1 and a CH3-based or CH2-based binding site for a second target. In a preferred embodiment, the antibody molecule is a human immunoglobulin G molecule, such as a human IgG1, IgG2, IgG3 or IgG4 molecule, more preferably a human IgG1 molecule.

Optionally, antibody or antigen-binding fragments thereof of the invention may have a second antigen-binding site located in a constant domain, preferably CH3 or CH2, of the antibody.

Alternatively or additionally, an antibody or antigen-binding fragment thereof of the invention may comprise a further CDR-based antigen-binding site (e.g., as formed by a VH and a VL) for a second or third target antigen. Thus, an antibody molecule, or antigen-binding fragment thereof, according to the invention may be a multispecific, preferably a bispecific, molecule comprising a second antigen-binding site.

The second antigen binding site, when present, may be a CH3-based or CH2-based antigen-binding site or a CDR-based antigen-binding site, and may bind an antigen such that the binding of said antigen is expected to be beneficial in the context of cancer treatment.

The antibody molecule may be a mAb²™ bispecific antibody. A mAb² bispecific antibody, as referred to herein, is an IgG immunoglobulin which includes a CDR-based antigen binding site in each of its variable regions and at least one antigen binding site in a constant domain of the antibody molecule.

In one embodiment, when the antibody or antigen-binding fragment thereof comprises a second antigen-binding site, e.g., a CH3-based, CH2-based or CDR-based antigen-binding site, the second antigen binding site may bind to a non-redundant and complementary inhibitory checkpoint molecule, such as CTLA-4, LAG-3, TIGIT, TIM-3, VISTA, CD73, CSF-1R, KIR. B7-H3, B7-H4, 2B4, NKG2A, CD47, SIRPa, BTLA, CCR4, CD200R, or TGFbeta.

The inhibition of the PD-1/PD-L1 axis and the stimulation of costimulatory molecules represent complementary strategies to enhance immune responses in human patients. The reversal of T cell exhaustion through checkpoint blockade may allow these cells to be activated more potently and to develop full anti-tumour activity. Thus, in another embodiment, an antibody or antigen-binding fragment thereof of the invention may comprise a second antigen-binding site, e.g., a CH3-based, CH2-based or CDR-based antigen-binding site, and the second antigen binding site may bind to, and be an agonist for, a costimulatory molecule expressed by T cells such as OX40, ICOS, CD40, HVEM. NKG2D, or TNFR2.

In a further embodiment, the antibody or antigen-binding fragment thereof of the invention may comprise a second antigen-binding site, e.g., a CH3-based, CH2-based or CDR-based antigen-binding site, and the second antigen-binding site may bind to a tumour associated antigen (TAA). Such antibody or antigen-binding fragment thereof is expected to result in tumour-specific T cell responses through localised immune activation. Examples of TAAs are c-Met, B7-H3, B7-H4, EGFR, HER-2, EPCAM, CEACAM, FAP, VEGF, MSLN, GPC3, CD38, CD19, and CD20.

As detailed above, infectious diseases show many parallels with oncology. The role of PD-L1 in immune regulation could be harnessed to maximise the immune response against pathogens. Immunomodulation in the context of treatment of infectious diseases is an emerging area of medicine and early reviews suggest that PD-L1 blockade may improve biological responses to infection, in particular, helping to counteract T-cell exhaustion, manage immune-mediated clearance, and generate long-term immunity (Wykes and Lewin, 2017).

In some infectious diseases, exaggerated pro-inflammatory responses and suboptimal antigen-specific T-cell activity are the causes of severe tissue damage (Rao et al., 2017). Without wishing to be bound by theory, it is thought that the use of an antibody or antigen-binding fragment thereof of the invention comprising a second antigen-binding site may find application in the treatment of these diseases by localising beneficial immunomodulatory activity to the pathogen environment.

Alternatively, use of an antibody or antigen-binding fragment thereof of the invention comprising a second antigen-binding site which binds to an immune cell target, either for agonism or antagonism, may result in increased T-cell specificity and activity.

Thus, in one embodiment, where the antibody or antigen-binding fragment thereof comprises a second antigen-binding site, the second antigen binding site may bind to an immune cell target, such as PD-1, PD-L2, CTLA-4, LAG-3, TIGIT, TIM3, OX40, CD40, ICOS, CD28, or CD80.

Alternatively, where the antibody or antigen-binding fragment thereof comprises a second antigen-binding site, the second antigen-binding site may bind to a pathogenic target, namely an antigen expressed by a human pathogen. The pathogen may be a virus, bacterium, fungus, or parasite. Preferably the pathogen is a virus, bacterium or fungus. More preferably, the pathogen is a virus or bacterium. Most preferably, the pathogen is a virus. Examples of viral antigens include proteins p24, gp120, and gp41 expressed by human immunodeficiency virus (HIV), hepatitis B surface antigen (HBsAg) expressed by hepatitis B virus (HBV), and hemagglutinin and neuraminidase expressed by influenza virus. Examples of bacterial antigens include Rv1733, Rv2389 and Rv2435n expressed by Mycobacterium tuberculosis.

In some embodiments, the second antigen-binding site of the antibody of the invention may not bind to OX40. In addition, or alternatively, the second antigen-binding site of an antibody of the invention may not bind to CD137. In addition, or alternatively, the second antigen-binding site of an antibody of the invention may not bind to CD27. In addition, or alternatively, the second antigen-binding site of an antibody of the invention may not bind to glucocorticoid-induced TNFR-related protein (GITR). In addition, or alternatively, the second antigen-binding site of an antibody of the invention may not bind to lymphocyte-activation gene 3 (LAG-3). In addition, or alternatively, the second antigen-binding site of an antibody of the invention may not bind to Inducible T-cell COStimulator (ICOS).

An antibody of the invention may be conjugated to an immune system modulator, cytotoxic molecule, radioisotope or detectable label. The immune system modulator may be a cytotoxic molecule, such as a cytokine.

The antibody molecule may be conjugated to a bioactive molecule or a detectable label. In this case, the antibody molecule may be referred to as a conjugate. Such conjugates find application in the treatment and/or diagnosis of diseases as described herein.

For example, the bioactive molecule may be an immune system modulator, such as a cytokine, preferably a human cytokine. For example, the cytokine may be a cytokine which stimulates T cell activation and/or proliferation. Examples of cytokines for conjugation to the antibody molecule include IL-2, IL-10, IL-12, IL-15, IL-21, GM-CSF and IFN-gamma.

Alternatively, the bioactive molecule may be a ligand trap, such as a ligand trap of a cytokine, e.g., of TGF-beta or IL-6.

Suitable detectable labels which may be conjugated to antibody molecules are known in the art and include radioisotopes such as iodine-125, iodine-131, yttrium-90, indium-111 and technetium-99; fluorochromes, such as fluorescein, rhodamine, phycoerythrin, Texas Red and cyanine dye derivatives for example, Cy7 and Alexa750; chromogenic dyes, such as diaminobenzidine; latex beads; enzyme labels such as horseradish peroxidase; phosphor or laser dyes with spectrally isolated absorption or emission characteristics; and chemical moieties, such as biotin, which may be detected via binding to a specific cognate detectable moiety, e.g., labelled avidin.

The antibody of the invention may be conjugated to the bioactive molecule or detectable label by means of any suitable covalent or non-covalent linkage, such as a disulphide or peptide bond. Where the bioactive molecule is a cytokine, the cytokine may be joined to the antibody molecule by means of a peptide linker. Suitable peptide linkers are known in the art and may be 5 to 25, 5 to 20, 5 to 15, 10 to 25, 10 to 20, or 10 to 15 amino acids in length.

In some embodiments, the bioactive molecule may be conjugated to the antibody by a cleavable linker. The linker may allow release of the bioactive molecule from the antibody at a site of therapy. Linkers may include amide bonds (e.g., peptidic linkers), disulphide bonds or hydrazones. Peptide linkers for example may be cleaved by site-specific proteases, disulphide bonds may be cleaved by the reducing environment of the cytosol and hydrazones may be cleaved by acid-mediated hydrolysis.

The conjugate may be a fusion protein comprising the antibody of the invention and the bioactive molecule. In this case the bioactive molecule may be conjugated to the antibody by means of a peptide linker or peptide bond. Where the antibody is a multichain molecule, such as where the antibody molecule is or comprises an Fcab or is a mAb², the bioactive molecule may be conjugated to one or more chains of the antibody molecule. For example, the bioactive molecule may be conjugated to one or both of the heavy chains of the mAb² molecule. Fusion proteins have the advantage of being easier to produce and purify, facilitating the production of clinical-grade material.

The invention also provides a nucleic acid or set of nucleic acids encoding an antibody or antigen-binding fragment of the invention, as well as a vector comprising such a nucleic acid or set of nucleic acids.

Where the nucleic acid encodes the VH and VL domain, or heavy and light chain, of an antibody molecule of the invention, the two domains or chains may be encoded on two separate nucleic acid molecules.

An isolated nucleic acid molecule may be used to express an antibody molecule of the invention. The nucleic acid will generally be provided in the form of a recombinant vector for expression. Another aspect of the invention thus provides a vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g., phage, or phagemid, as appropriate.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. A preferred host cell is a mammalian cell, such as a CHO, NS0, or HEK cell, for example a HEK293 cell.

A recombinant host cell comprising a nucleic acid or the vector of the invention is also provided. Such a recombinant host cell may be used to produce an antibody of the invention. Thus, also provided is a method of producing an antibody of the invention, the method comprising culturing the recombinant host cell under conditions suitable for production of the antibody. The method may further comprise a step of isolating and/or purifying the antibody molecule.

Thus the invention provides a method of producing an antibody molecule of the invention comprising expressing a nucleic acid encoding the antibody molecule in a host cell and optionally isolating and/or purifying the antibody molecule thus produced. Methods for culturing host cells are well-known in the art. Techniques for the purification of recombinant antibody molecules are well-known in the art and include, for example HPLC, FPLC or affinity chromatography, e.g., using Protein A or Protein L. In some embodiments, purification may be performed using an affinity tag on antibody molecule. The method may also comprise formulating the antibody molecule into a pharmaceutical composition, optionally with a pharmaceutically-acceptable excipient or other substance as described below.

The antibodies of the invention are expected to find application in therapeutic applications, in particular therapeutic applications in humans, such as cancer treatment and the treatment of infectious diseases. Thus, also provided is a composition such as a pharmaceutical composition comprising an antibody molecule according to the invention and an excipient, such as a pharmaceutically-acceptable excipient.

The invention further provides an antibody molecule of the invention, for use in a method of treatment. Also provided is a method of treating a patient, wherein the method comprises administering to the patient a therapeutically-effective amount of an antibody molecule according to the invention. Further provided is the use of an antibody molecule according to the invention for use in the manufacture of a medicament. A patient, as referred to herein, is preferably a human patient.

The invention also provides an antibody molecule of the invention, for use in a method of treating cancer in a patient. Also provided is a method of treating cancer in a patient, wherein the method comprises administering to the patient a therapeutically-effective amount of an antibody molecule according to the invention. Further provided is the use of an antibody molecule according to the invention for use in the manufacture of a medicament for the treatment of cancer in a patient. The treatment may further comprise administering to the patient a second anti-cancer agent and/or therapy, such as an anti-tumour vaccine and/or a chemotherapeutic agent. The second anti-cancer agent and/or therapy may be administered to the patient simultaneously, separately, or sequentially to the antibody molecule of the invention.

In another aspect, the invention relates to an antibody that binds to PD-L1 for use in a) treating cancer, b) delaying progression of cancer, c) prolonging the survival of a patient suffering from cancer, or d) stimulating a cell-mediated immune response.

The invention also provides an antibody of the invention, for use in a method of treating an infectious disease in a patient. Also provided is a method of treating an infectious disease in a patient, wherein the method comprises administering to the patient a therapeutically-effective amount of an antibody according to the invention. Further provided is the use of an antibody according to the invention for use in the manufacture of a medicament for the treatment of an infectious disease in a patient. The treatment may further comprise administering to the patient a second agent and/or therapy for the treatment of the infectious disease. The second agent and/or therapy may be administered to the patient simultaneously, separately, or sequentially to the antibody or antigen-binding fragment thereof or antibody molecule of the invention.

The antibody molecules as described herein may thus be useful for therapeutic applications, in particular in the treatment of cancer. In addition, the antibody molecules are expected to be useful in the treatment of infectious diseases, such as persistent infectious diseases.

An antibody molecule as described herein may be used in a method of treatment of the human or animal body. Related aspects of the invention provide;
(i) an antibody molecule described herein for use as a medicament,
(ii) an antibody molecule described herein for use in a method of treatment of a disease or disorder,
(iii) the use of an antibody molecule described herein in the manufacture of a medicament for use in the treatment of a disease or disorder; and,
(iv) a method of treating a disease or disorder in an individual, wherein the method comprises administering to the individual a therapeutically effective amount of an antibody molecule as described herein.

The individual may be a patient, preferably a human patient.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e., prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

A method of treatment as described may be comprise administering at least one further treatment to the individual in addition to the antibody molecule. The antibody molecule described herein may thus be administered to an individual alone or in combination with one or more other treatments. Where the antibody molecule is administered to the individual in combination with another treatment, the additional treatment may be administered to the individual concurrently with, sequentially to, or separately from the administration of the antibody molecule. Where the additional treatment is administered concurrently with the antibody molecule, the antibody molecule and additional treatment may be administered to the individual as a combined preparation. For example, the additional therapy may be a known therapy or therapeutic agent for the disease to be treated.

Whilst an antibody molecule may be administered alone, antibody molecules will usually be administered in the form of a pharmaceutical composition, which may comprise at least one component in addition to the antibody molecule. Another aspect of the invention therefore provides a pharmaceutical composition comprising an antibody molecule as described herein. A method comprising formulating an antibody molecule into a pharmaceutical composition is also provided.

Pharmaceutical compositions may comprise, in addition to the antibody molecule, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g., by injection, the pharmaceutical composition comprising the antibody molecule may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g., Zn-protein complexes); and/or non-ionic surfactants, such as TWEEN™, PLURONICS™ or polyethylene glycol (PEG).

In some embodiments, antibody molecules may be provided in a lyophilised form for reconstitution prior to administration. For example, lyophilised antibody molecules may be re-constituted in sterile water or saline prior to administration to an individual.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners. Prescription of treatment, e.g., decisions on dosage etc., is within the responsibility of general practitioners and other medical doctors, and may depend on the severity of the symptoms and/or progression of a disease being treated. Appropriate doses of antibody molecules are well known in the art (Ledermann et al., 1991; Bagshawe et al., 1991). Specific dosages indicated herein, or in the Physician's Desk Reference (2003) as appropriate for an antibody molecule being administered, may be used. A therapeutically effective amount or suitable dose of an antibody molecule can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antibody molecule.

A typical antibody dose is in the range 100 μg to 1 g for systemic applications, and 1 μg to 1 mg for topical applications. An initial higher loading dose, followed by one or more lower doses, may be administered. This is a dose for a single treatment of an adult individual, which may be proportionally adjusted for children and infants, and also adjusted for other antibody formats in proportion to molecular weight.

Treatments may be repeated at daily, twice-weekly, weekly or monthly intervals, at the discretion of the physician. The treatment schedule for an individual may be dependent on the pharmacokinetic and pharmacodynamic properties of the antibody composition, the route of administration and the nature of the condition being treated.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Suitable formulations and routes of administration are described above.

In a preferred embodiment, an antibody molecule as described herein may be for use in a method of treating cancer.

Cancer may be characterised by the abnormal proliferation of malignant cancer cells. Where a particular type of cancer, such as breast cancer, is referred to, this refers to an abnormal proliferation of malignant cells of the relevant tissue, such as breast tissue. A secondary cancer which is located in the breast but is the result of abnormal proliferation of malignant cells of another tissue, such as ovarian tissue, is not a breast cancer as referred to herein but an ovarian cancer.

The cancer may be a primary or a secondary cancer. Thus, an antibody molecule as described herein may be for use in a method of treating cancer in an individual, wherein the cancer is a primary tumour and/or a tumour metastasis.

A tumour of a cancer to be treated using an antibody molecule as described herein may comprise cells that express PD-L1, e.g., on their cell surface. In one embodiment, the tumour may have been determined to comprise cells that express PD-L1. Methods for determining the expression of an antigen on a cell surface are known in the art and include, for example, flow cytometry.

For example, the cancer to be treated using an antibody molecule as described herein may be selected from the group consisting of leukaemias, such as acute myeloid leukaemia (AML), chronic myeloid leukaemia (CML), acute lymphoblastic leukaemia (ALL) and chronic lymphocytic leukaemia (CLL); lymphomas, such as Hodgkin lymphoma, non-Hodgkin lymphoma and multiple myeloma; and solid cancers, such as sarcomas (e.g., soft tissue sarcomas), skin cancer (e.g., Merkel cell carcinoma), melanoma, bladder cancer (e.g., urothelial carcinoma), brain cancer (glioblastoma multiforme), breast cancer, uterine/endometrial cancer, ovarian cancer (e.g., ovarian serous cystadenoma), prostate cancer, lung cancer (e.g., non-small cell lung carcinoma (NSCLC) and small cell lung cancer (SCLC)), colorectal cancer (e.g., colorectal adenocarcinoma), cervical cancer (e.g., cervical squamous cell cancer and cervical adenocarcinoma), liver cancer (e.g., hepatocellular carcinoma), head and neck cancer (e.g., head and neck squamous-cell carcinoma), oesophageal cancer, pancreatic cancer, renal cancer (e.g., renal cell cancer), adrenal cancer, stomach cancer (e.g., stomach adenocarcinoma), testicular cancer, cancer of the gall bladder and biliary tracts (e.g., cholangiocarcinoma), thyroid cancer, thymus cancer, bone cancer, and cerebral cancer.

In a preferred embodiment, the cancer to be treated using an antibody molecule as described herein is a solid cancer. More preferably, the cancer to be treated using an antibody molecule as described herein is a solid cancer selected from the group consisting of: sarcoma, melanoma, bladder cancer, brain cancer, breast cancer, ovarian cancer, uterine/endometrial cancer, prostate cancer, lung cancer, colorectal cancer, cervical cancer, liver cancer, head and neck cancer, pancreatic cancer, renal cancer and stomach cancer.

In the context of cancer, treatment may include inhibiting cancer growth, including complete cancer remission, and/or inhibiting cancer metastasis, as well as inhibiting cancer recurrence. Cancer growth generally refers to any one of a number of indices that indicate change within the cancer to a more developed form. Thus, indices for measuring an inhibition of cancer growth include a decrease in cancer cell survival, a decrease in tumour volume or morphology (for example, as determined using computed tomographic (CT), sonography, or other imaging method), a delayed tumour growth, a destruction of tumour vasculature, improved performance in delayed hypersensitivity skin test, an increase in the activity of anti-cancer immune cells or other anti-cancer immune responses, and a decrease in levels of tumour-specific antigens. Activating or enhancing immune responses to cancerous tumours in an individual may improve the capacity of the individual to resist cancer growth, in particular growth of a cancer already present in the subject and/or decrease the propensity for cancer growth in the individual.

In the context of cancer treatment, an antibody molecule as described herein may be administered to an individual in combination with another anti-cancer therapy or therapeutic agent, such as an anti-cancer therapy or therapeutic agent which has been shown to be suitable, or is expected to be suitable, for the treatment of the cancer in question. For example, the antibody molecule may be administered to the individual in combination with a chemotherapeutic agent, radiotherapy, an immunotherapeutic agent, an anti-tumour vaccine, an oncolytic virus, an adoptive cell transfer (ACT) therapy (such as adoptive NK cell therapy or therapy with chimeric antigen receptor (CAR) T-cells, autologous tumour infiltrating lymphocytes (TILs), or gamma/delta T cells, or an agent for hormone therapy.

Without wishing to be bound by theory, it is thought that the antibody molecule described herein may act as an adjuvant in anti-cancer therapy. Specifically, it is thought that administration of the antibody molecule to an in individual in combination with chemotherapy and/or radiotherapy, or in combination with an anti-tumour vaccine, for example, will trigger a greater immune response against the cancer than is achieved with chemotherapy and/or radiotherapy, or with an anti-tumour vaccine, alone.

One or more chemotherapeutic agents for administration in combination with an antibody of the invention as described herein may be selected from the group consisting of: taxanes, cytotoxic antibiotics, tyrosine kinase inhibitors, PARP inhibitors, B-Raf enzyme inhibitors, MEK inhibitors, c-MET inhibitors, VEGFR inhibitors, PDGFR inhibitors, alkylating agents, platinum analogues, nucleoside analogues, antifolates, thalidomide derivatives, antineoplastic chemotherapeutic agents and others. Taxanes include docetaxel, paclitaxel and nab-paclitaxel; cytotoxic antibiotics include actinomycin, bleomycin, and anthracyclines such as doxorubicin, mitoxantrone and valrubicin; tyrosine kinase inhibitors include erlotinib, gefitinib, axitinib, PLX3397, imatinib, cobemitinib and trametinib; PARP inhibitors include piraparib; B-Raf enzyme inhibitors include vemurafenib and dabrafenib; alkylating agents include dacarbazine, cyclophosphamide and temozolomide; platinum analogues include carboplatin, cisplatin and oxaliplatin; nucleoside analogues include azacitidine, capecitabine, fludarabine, fluorouracil and gemcitabine; antifolates include methotrexate and pemetrexed. Other chemotherapeutic agents suitable for use in the present invention include defactinib, entinostat, eribulin, irinotecan and vinblastine.

Preferred therapeutic agents for administration with an antibody molecule as described herein are doxorubicin, mitoxantrone, cyclophosphamide, cisplatin, and oxaliplatin.

A radiotherapy for administration in combination with an antibody molecule as described herein may be external beam radiotherapy or brachytherapy.

An immunotherapeutic agent for administration in combination with an antibody molecule as described herein may be a therapeutic antibody molecule, nucleic acid, cytokine, or cytokine-based therapy. For example, the therapeutic antibody molecule may bind to an immune regulatory molecule, e.g., an inhibitory checkpoint molecule or an immune costimulatory molecule, a receptor of the innate immune system, or a tumour antigen, e.g., a cell surface tumour antigen or a soluble tumour antigen. Examples of immune regulatory molecules to which the therapeutic antibody molecule may bind include CTLA-4, LAG-3, TIGIT, TIM-3, VISTA, PD-1, CD47, CD73, CSF-1R, KIR, OX40, CD40, HVEM, IL-10 and CSF-1. Examples of receptors of the innate immune system to which the therapeutic antibody molecule may bind include TLR1, TLR2, TLR4, TLR5, TLR7, TLR9, RIG-I-like receptors (e.g., RIG-I and MDA-5), and STING. Examples of tumour antigens to which the therapeutic antibody molecule may bind include HER2, EGFR. CD20 and TGF-beta.

The nucleic acid for administration in combination with an antibody molecule as described herein may be a siRNA.

The cytokines or cytokine-based therapy may be selected from the group consisting of: IL-2, prodrug of conjugated IL-2, GM-CSF, IL-7, IL-12, IL-9, IL-15, IL-18, IL-21, and type I interferon.

Anti-tumour vaccines for the treatment of cancer have both been implemented in the clinic and discussed in detail within scientific literature (such as Rosenberg S. Development of Cancer Vaccines. ASCO Educational Book Spring: 60-62 (2000)). This mainly involves strategies to prompt the immune system to respond to various cellular markers expressed by autologous or allogenic cancer cells by using those cells as a vaccination method, both with or without granulocyte-macrophage colony-stimulating factor (GM-CSF). GM-CSF provokes a strong response in antigen presentation and works particularly well when employed with said strategies.

The chemotherapeutic agent, radiotherapy, immunotherapeutic agent, anti-tumour vaccine, oncolytic virus, ACT therapy, or agent for hormone therapy is preferably a chemotherapeutic agent, radiotherapy, immunotherapeutic agent, anti-tumour vaccine, oncolytic virus, ACT therapy, or agent for hormone therapy for the cancer in question, i.e. a chemotherapeutic agent, radiotherapy, immunotherapeutic agent, anti-tumour vaccine, oncolytic virus, ACT therapy, or agent for hormone therapy which has been shown to be effective in the treatment of the cancer in question. The selection of a suitable chemotherapeutic agent, radiotherapy, immunotherapeutic agent, anti-tumour vaccine, oncolytic virus, ACT therapy, or agent for hormone therapy which has been shown to be effective for the cancer in question is well within the capabilities of the skilled practitioner.

In some embodiments for potential therapeutic use, an antibody that does not activate effector functions is preferred.

IgG4 has been used but this sub-class to undergo Fab-arm exchange, where heavy chains can be swapped between IgG4 in vivo. Due to their lack of effector functions, IgG4 antibodies represent the preferred IgG subclass for receptor blocking without cell depletion. IgG4 molecules can exchange half-molecules in a dynamic process termed Fab-arm exchange. This phenomenon can occur between therapeutic antibodies and endogenous IgG4. The S228P mutation has been shown to prevent this recombination process allowing the design of less unpredictable therapeutic IgG4 antibodies.

The CH2 domain is known bind to Fcγ receptors and complement. Binding of the CH2 domain to Fcγ receptors is required antibody-dependent cell-mediated cytotoxicity (ADCC), while binding to complement is required complement-dependent cytotoxicity (CDC). The CH2 domain of the antibody molecule preferably comprise one or more mutations that reduce or abrogate binding of the CH2 domain to one or more Fcγ receptors, such as FcγRI, FcγRIIa, FcγRIIb, FcγRIII, and/or to complement. The inventors postulate that reducing or abrogating binding to Fcγ receptors will decrease or eliminate ADCC mediated by the antibody molecule. Similarly, reducing or abrogating binding to complement is expected to reduce or eliminate CDC mediated by the antibody molecule. Without wishing to be bound by theory, this is expected to reduce or avoid liver inflammation when the antibody molecule is administered to a patient. In addition, reducing or abrogating binding to Fcγ receptors is expected to be useful where the antibody molecule comprises a second antigen-binding site for an immune cell antigen as described herein, where ADCC and/or CDC-mediated killing of immune cells bound by the antibody molecule should be avoided. Mutations to decrease or abrogate binding of the CH2 domain to one or more Fcγ receptors and/or complement are known in the art (Wang et al., 2018). These mutations include the “LALA mutation” described in Bruhns et al., 2009 and Hezareh et al., 2001, which involves substitution of the leucine residues at IMGT positions 1.3 and 1.2 of the CH2 domain with alanine (L1.3A and L1.2A). Alternatively, the generation of a-glycosyl antibodies through mutation of the conserved N-linked glycosylation site by mutating the asparagine (N) at IMGT position 84.4 of the CH2 domain to alanine, glycine or glutamine (N84.4A, N84.4G or N84.4Q) is also known to decrease IgG1 effector function (Wang et al., 2018). As a further alternative, complement activation (C1q binding) and ADCC are known to be reduced through mutation of the proline at IMGT position 114 of the CH2 domain to alanine or glycine (P114A or P114G) (Idusogie et al., 2000; Klein et al., 2016). These mutations may also be combined in order to generate antibody molecules with further reduced or no ADCC or CDC activity.

Thus, the antibody molecule may comprise a CH2 domain, wherein the CH2 domain preferably comprises:
(i) alanine residues at positions 1.3 and 1.2; and/or
(ii) an alanine or glycine at position 114; and/or
(iii) an alanine, glutamine or glycine at position 84.4;
wherein the amino acid residue numbering is according to the IMGT numbering scheme.

In a preferred embodiment, the antibody molecule comprises a CH2 domain, wherein the CH2 domain comprises:
(i) an alanine residue at position 1.3; and
(ii) an alanine residue at position 1.2;
wherein the amino acid residue numbering is according to the IMGT numbering scheme.

In an alternative preferred embodiment, the antibody molecule comprises a CH2 domain, wherein the CH2 domain comprises:
(i) an alanine residue at position 1.3;
(ii) an alanine residue at position 1.2; and
(iii) an alanine at position 114;
wherein the amino acid residue numbering is according to the IMGT numbering scheme.

IgG naturally persists for a prolonged period in serum due to FcRn-mediated recycling, giving it a typical half life of approximately 21 days. To prolong half life the pH dependent interaction of the Fc domain with FcRn has been engineered to increase affinity at pH 6.0 while retaining minimal binding at pH 7.4. The mutations T250Q/M428L, conferred an approximately 2-fold increase in IgG half-life in rhesus monkeys. The M252Y/S254T/T256E variant (dubbed YTE), conferred an approximately 4-fold increase in IgG half-life in cynomolgus monkeys. A longer half-life is desirable in some circumstances to decrease the frequency of administration whilst maintaining or improving efficacy of the administered antibody. Antibodies of the invention may be provided as half-life extended variants, engineered to extend half-life in vivo serum following administration, thus antibodies of the invention may be provided as T250Q/M428L or M252Y/S254T/T256E variants.

The antibody molecules of the invention may be useful in the detection of PD-L1, in particular in the detection of cells comprising PD-L1 at their cell surface, i.e. cells expressing cell-surface bound PD-L1. The cells may be immune cells, such as CD8⁺ T cells, CD4⁺ T cells, Treg cells, B cells, NK cells, NKT cells, dendritic cells, or TILs, but preferably are CD8⁺ T cells or TILs.

Thus, the present invention relates to the use of an antibody molecule for detecting the presence of PD-L1, preferably the presence of cells comprising PD-L1 at their cell surface, in a sample. The antibody molecule may be conjugated to a detectable label as described elsewhere herein.

Also provided is an in vitro method of detecting PD-L1, wherein the method comprises incubating the antibody molecule with a sample of interest, and detecting binding of the antibody molecule to the sample, wherein binding of the antibody to the sample indicates the presence of PD-L1. Binding of the antibody molecule to a sample may be detected using an ELISA, for example.

In a preferred embodiment, the present invention relates to an in vitro method of detecting cells comprising PD-L1 at their cell surface, wherein the method comprises incubating the antibody molecule with a cell sample of interest, and determining binding of the antibody molecule to cells present in the sample, wherein binding of the antibody to cells present in sample indicates the presence of cells comprising PD-L1 at their cell surface. Methods for detecting binding of an antibody molecule to cells are known in the art and include ELISAs, and flow-cytometry.

The cell sample of interest may be a tumour sample obtained from an individual.

The antibody molecules of the invention may thus be useful in the detection or diagnosis of disease or disorder, in particular the detection or diagnosis of cancer. The cancer may be a cancer which can be treated with an antibody molecule of the invention as described herein.

Related aspects of the invention thus provide;
(i) an antibody molecule described herein for use as a diagnostic,
(ii) an antibody molecule described herein for use in a method of detecting or diagnosing a disease or disorder, such as cancer,
(iii) the use of an antibody molecule described herein in the manufacture of a diagnostic product for use in the detection or diagnosis of a disease or disorder;
(iv) a method of detecting or diagnosing a disease or disorder in an individual; and
(v) a kit for use in a method of detecting or diagnosing a disease or disorder in an individual, the kit comprising an antibody molecule as described herein.

Further aspects and embodiments of the invention will be apparent to those skilled in the art given the present disclosure, including the following experimental exemplification.

All documents mentioned in this specification are incorporated herein by reference in their entirety.
“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures provided herein.

### LIST OF FIGURES

FIG. 1: Mixed Leukocyte Reaction Assay. The functional activity of the anti-PD-L1 kappa clones, G1/894_8_E05, G1/887_4_E12 and G1/887_4_G12, was tested in a mixed leukocyte reaction assay. All anti-PD-L1 mAbs showed potent activity with EC₅₀ values lower than 0.030 nM. No activity was observed for the negative control G1AA/4420.

FIG. 2: DO11.10 mouse T cell activation assay. The functional activity of the anti-PD-L1 kappa clones, G1/894_8_E05, G1/887_4_E12 and G1/887_4_G12, towards mouse PD-L1 was tested in a T cell assay with LK35.2 overexpressing mouse PD-L1 and DO11.10 T cells. All anti-PD-L1 mAbs showed potent activity with low nanomolar EC₅₀ values. No activity was observed for the negative control G1AA/4420.

FIG. 3: Pharmacokinetics of anti-PD-L1 mAbs in non-tumour bearing mice.

FIG. 4: Mixed Leukocyte Reaction Assay. The functional activity of the anti-PD-L 1 kappa clones, G1AA/E05v2, G1AA/E12v2 and G1AA/G12v2 was tested in a mixed leukocyte reaction assay. All anti-PD-L1 mAbs showed potent activity with EC₅₀ values below 0.055 nM. No activity was observed for the negative control G1AA/4420.

### EXAMPLES

The aim of these experiments was to generate an anti-human-PD-L 1 mAb that was cross-reactive with mouse and/or cynomolgus PD-L1 and which was a potent inhibitor (blocker) of PD-1/PD-L1 activity.

### Example 1: Isolation of Naïve Anti-PD-L1 Binding mAb: 280_02_G02

### 1.1 Antigens: CD4 and Fc Tagged Human and Mouse PD-L1

Human and mouse PD-L1 antigens with fusion proteins were generated for use in antibody selections and screening. Antigens were expressed with either a monomeric C-terminal rat CD4, domains 3 and 4 (rCd4) tag (Brown and Barclay, 1994) or a dimeric human IgG1 Fc domain (resulting in hPD-L1-rCD4-His (SEQ ID NO: 79), hPD-L1-Fc-His (SEQ ID NO: 80), mPD-L1-rCD4-His (SEQ ID NO: 81) and mPD-L1-Fc-His (SEQ ID NO: 82). The production of antigens in two different formats enabled the elimination of tag binders during sequential antibody phage display pannings. Expression plasmids encoding the antigens were transfected into HEK293 cells as described by Chapple et al., 2006. Supernatants were harvested 5 days after transfection and the secreted antigens were purified by Ni-NTA sepharose affinity chromatography (Schofield et al., 2007). Biotinylated antigen was prepared using EZ-link Sulfo-NHS-Biotin reagent (Thermo Fisher Scientific, product code 21326) following the manufacturer's recommendations. The biotinylation reaction product was gel filtered and the monomeric fraction was collected. The monomeric fraction was used for all solution-phase phage-display selections. The average number of biotins per molecule was 1 to 3 biotins per PD-L1 monomer as determined using Fluorescence Biotin Quantitation kit (Thermo Fisher Scientific, product code 46610).

### 1.2 Selections

### 1.2.1 Library Design

The “IONTAS 1” human antibody phage display library (IONTAS Ltd.) was employed to select for anti-PD-L1 clones. The antibody genes used to construct the IONTAS 1 library were derived from human lymphocytes (42 buffy coat donations) and one tonsil tissue sample. Both the buffy coats and tonsil tissue were obtained under Local Research Ethical Committee approval.

### 1.2.2 Naïve Solid Phase Selection

Three rounds of solid phase selections were performed with the IONTAS 1 antibody phage display library using antigen that was directly coated onto polystyrene Nunc tubes as described by Schofield et al., 2007. The first, second and third selection rounds employed human PD-L1-Fc-His (SEQ ID NO: 80), mouse PD-L1-rCD4-His (SEQ ID NO: 81) and human PD-L1-Fc-His (SEQ ID NO: 80), respectively. In the first round, Nunc Maxisorp Immunotubes (Thermo Scientific. 444202) were coated overnight with 10 μg/ml of human PD-L1-Fc-His for direct selection. The next day the tubes were rinsed 2x in PBS and then blocked by filling to the top with PBS 2% Marvel/PBS (MPBS) and incubated for 1 hour, then the tubes were washed three times with PBS. Also, the IONTAS 1 antibody phage display library (500 μl) was blocked with 4% MPBS (500 μl) for 1 hour. To each antigen coated immunotube 2% MPBS (179.25 μl), Fc-His (10.75 μl, 2.8 mg/ml) and blocked IONTAS 1 antibody phage display library (110 μl, 2×10¹² colony forming units, 2% MPBS) was added. Fc-His was added to the selection to remove anti-Fc binders binding to the solid phase immobilized Fc tagged antigen. The antibody phage display library was allowed to bind to the directly-immobilized antigen for 1.5 hours at room temperature. After this time, the immuno-tubes were washed 6 times with PBS-T (PBS, pH7.4, 0.1% Tween™-20), then washed 6 times with PBS. Bound phages were eluted and propagated using standard phage recovery procedures. The second round of antibody phage display selection was performed as above, except that mouse PD-L1-rCd4-His (SEQ ID NO: 81) was used to coat the Nunc immunosorb tube, rCd4-His was used instead of Fc-His for the de-selection and the round 1 output phage, selected against human PD-L1-Fc-His, was used as the input phage population. The third round with human PD-L1-Fc-His was performed exactly as round 1.

### 1.2.3 Chain Shuffling and Solution Phase Selections

The selected variable heavy (VH) anti-PD-L1 antibody population was shuffled with a naïve variable light (VL) antibody population as described by Dyson et al., 2011, and this shuffled, rescued, antibody-phage-display population was employed in solution phase selections. Briefly, panning was performed with human PD-L1-rCd4-His (SEQ ID NO: 79) (10 nM), human PD-L1-rCd4-His (SEQ ID NO: 79) (200 pM) and mouse PD-L1-rCd4-His (SEQ ID NO: 81) (10 nM) at rounds 1, 2 and 3, respectively, and this resulted in an output anti-PD-L1 scFv population termed “Selection 280”. This scFv population contained human and mouse anti-PD-L1 binding scFvs, as determined by a phage polyclonal ELISA performed as described by Dyson et al., 2011, and displayed minimal cross-reactivity with human PD1 or with rCd4 or Fc tags.

### 1.3 Screening: ELISA, Recombinant Blocking Assay, Cell-Based Blocking Assay

1.3.1 Monoclonal scFv ELISA

The Selection 280 scFv population from Example 1.2.3 was screened by ELISA to identify the clones which bound best to human PD-L1. The scFv population was subcloned into the soluble scFv vector pSANG10 and E. coli cultures containing soluble scFvs were prepared as described (Martin et al., 2006; Studier, 2005). Soluble scFv were then used in a monoclonal ELISA with immobilised human PD-L1-rCd4-His (SEQ ID NO: 79). Briefly, Nunc Maxisorp plates (Thermo Fisher Scientific, 437111) were coated with human PD-L1-rCd4-His (SEQ ID NO: 79) (5 μg/ml, PBS) overnight, blocked with 2% MPBS for 1 hour and the E. coli culture supernatant (1:2 dilution with 2×2% MPBS) was added and scFv were allowed to bind for 1 hour at room temperature. Bound scFv were detected with anti-FLAG M2 antibody (Sigma, F1804) labelled with europium. A total of 470 clones were screened and this resulted in the identification of 346 anti-PD-L1 clones with a binding signal for PD-L1 at least 10-fold above background compared with “empty” blocked wells containing no antigens. The 192 best anti-human PD-L1 clones assessed by primary ELISA signal were selected for further analysis.

1.3.2 Screening scFv in ELISA-Based PD-L1/PD-1 Blocking Assay

To identify clones that blocked the interaction between PD-L1 and PD1, an ELISA was performed to screen for blocking scFvs. Briefly, nunc maxisorp plates (437111, Thermo Fisher Scientific) were coated with anti-rCd4 (domains 3 and 4) antibody (MCA1022, OX-68, Bio-Rad) overnight, blocked with 3% MPBS and incubated with human PD1-rCd4-His (SEQ ID NO: 79) (5 μg/ml in 3% MPBS) for 1 hour at room temperature. Biotinylated human PD-L1-Fc-His (SEQ ID NO: 79) (50 μl, 0.2 nM), was pre-mixed with E. coli culture supernatant containing scFv. The Nunc 96-well plates were washed 3 times with PBS, 0.1% Tween™-20 (PBS-T) and 3 times with PBS, then the human PD-L1-Fc-His/scFv mix was added and incubated for 1 hour at room temperature. The plates were washed and bound human PD-L1-Fc-His was detected using goat-anti-Fc-biotin (Jackson ImmunoResearch, 109-065-098, Laboratories, 0.1 μg/ml, 3% MPBS) and Streptavidin-Europium (Perkin Elmer, 1244-360) followed by DELFIA enhancement solution (Perkin Elmer, 4001-0010). Of 192 clones screened, 183 displayed at least 90% blocking activity compared with the medium control. The 183 anti-PD-L1 scFv clones identified were screened further for mouse PD-L1 cross-reactivity in a primary ELISA, as described in Example 1.3.1, but using immobilised mouse PD-L1-rCD4-His instead of human PD-L1. This identified 50 mouse cross-reactive anti-PD-L1 clones, which were candidates for conversion to IgG1 format.

1.3.3 Conversion of Blocking Anti-PD-L1 scFv Clones to IgG1 Format

The anti-PD-L1 scFv clones which blocked the interaction between PD-L1 and PD1 were converted to IgG1 format by sub-cloning the VL and VH genes into the IgG1 expression plasmid pINT3-IgG1 and were expressed in HEK293 at 4 ml scale as described by Chapple et al., 2006. The antibodies were batch affinity purified with Protein A sepharose beads (PC-A100) and Proteus “1-step batch” midi-spin columns (Generon, GEN-1SB08) according to the manufacturer's instructions. Dialysis of the purified antibodies was performed with GeBAflex maxi tubes, with an 8 kDa cut-off (Generon, D045). If necessary, the antibodies were concentrated to 2 μM by ultrafiltration.

### 1.3.4 Screening for PD-L1/PD-1 Blocking Activity in Jurkat-NFAT Reporter Co-Culture Assay

The functional activity of the purified anti-PD-L1 mAbs was then assessed in a co-culture reporter assay screen. This screen was performed using the GloResponse NFAT-luc2/PD-1 stable Jurkat cell line (Promega, CS187102) and Thaw-and-Use PD-L1 cells (Promega, CS178103) in accordance with the manufacturer's instructions. The PD-L1 cells were plated in HAM'S-F12 medium containing 10% FBS. The next day PD-1 Jurkat reporter cells (Promega, CS187102) were resuspended in assay medium (90% RPMI1640, 1% FBS). Media was removed from the plate containing adhered PD-L1 cells and 40 μl of assay media containing different antibodies at a 2× concentration (200 nM) followed by 40 μl of the PD-1 cell mix was added to the adhered cells. The plate was incubated for 6 hours at 37° C., 5% CO₂. BioGlo reagent (Promega, G7940, 80 μl) was added to each well and the luciferase output was read using a BMG pherastar plate reader. This identified antibody G1/280_02_G02 as capable of blocking the interaction of PD-L1 with PD1 in a co-culture assay, as determined by increased luciferase activity compared to controls with no antibody. This activity was confirmed in a dose-response co-culture assay (doubling concentration range: 200 to 1.56 nM) resulting in a calculated half maximal effective concentration (EC₅₀) of 4.2 nM.

### 1.4 Sequence Optimisation

Preliminary analysis of the sequence of the G1/280_02_G02 antibody resulted in the identification of a potential deamidation site in the VH-CDR2, specifically an NG motif at Kabat positions 54 to 55. As deamidation at this site could potentially affect binding, variant clones were produced in which the NG motif was changed to either NA, NS, SG or GG. These modifications did not result in any significant reduction in affinity for recombinant PD-L1 or potency in PD-L1 blocking activity, and the variant clone containing the NS modification, designated G1/280_02_G02_NS, was chosen for use in a light-chain shuffle.

### 1.5 Summary of Naïve Selections

The phage selections strategies employed identified more than 50 anti-human PD-L1 binding clones with potent in vitro PD-1/PD-L1 blocking activity as well as mouse PD-L1 cross-reactivity.

In particular, G1/280_02_G02 showed potent activation in a cell-based PD-L1 reporter assay and was therefore selected for further optimisation.

### Example 2: Chain Shuffling to Generate Kappa Light Chain-Containing Anti-PD-L1 Clones

The G1/280_02_G02_NS antibody possesses a lambda light chain. As most monoclonal antibodies used in a clinical context to date have kappa light chains (Jain et al., 2017), it was sought, by the use of a chain-shuffling campaign, to generate clones comprising the heavy chain of the G1/280_02_G02_NS antibody but paired with kappa light chains, which retained affinity for human PD-L1 and mouse cross-reactivity. The IONTAS™ kappa-light-chain library in the phage display plasmid plONTAS-1 (kappa-library) was used to prepare a light-chain-shuffled library of scFv clones comprising the heavy chain of the G1/280_02_G02_NS antibody coupled with light chain variants.

### 2.1 Phage Selections and Screening Strategy

A number of phage-display solution selections were performed in three rounds using biotinylated human PD-L1-rCD4-His (SEQ ID NO: 79) and mouse PD-L1-rCD4-His antigens (SEQ ID NO: 81). The selections were performed by decreasing the antigen concentrations in every round (varying from 100 to 0.02 nM) and for each round of selection a “no-antigen” control was used. Details of the selections are shown in Table 2.

TABLE 2

| Selection no. | Antigen | Input library | Round |
|---|---|---|---|
| 866 | hPD-L1; 100 nM | Chain shuffled library | 1 |
| 867 | hPD-L1; 10 nM | Chain shuffled library | 1 |
| 868 | hPD-L1; 1 nM | Chain shuffled library | 1 |
| 869 | hPD-L1; 1 nM | Output of 867 | 2 |
| 870 | hPD-L; 0.2 nM | Output of 867 | 2 |
| 871 | hPD-L1; 0.1 nM | Output of 868 | 2 |
| 872 | hPD-L1; 0.02 nM | Output of 868 | 2 |
| 887 | hPD-L1; 0.1 nM | Output of 869 | 3 |
| 888 | mPD-L1; 1.0 nM | Output of 869 | 3 |
| 889 | mPD-L1; 0.1 nM | Output of 869 | 3 |
| 890 | hPD-L1; 0.02 nM | Output of 870 | 3 |
| 891 | mPD-L1; 1.0 nM | Output of 870 | 3 |
| 892 | mPD-L1; 0.1 nM | Output of 870 | 3 |
| 893 | mPD-L1; 1.0 nM | Output of 872 | 3 |
| 894 | mPD-L1; 0.1 nM | Output of 872 | 3 |

Six selection outputs were selected for screening, two from round 2 (nos. 871 and 872) and four from round 3 (nos. 887, 890, 891 and 894) using the soluble scFv expression system as described in section 1.3.1. A total of 1692 soluble scFv clones were screened for binding to immobilised antigen in ELISA (hu-PD-L1-rCD4-His (SEQ ID NO: 79) antigen coated at 3 μg/mL in Dulbecco PBS, 50 μl, onto Maxisorb plates) using the assay described in Example 1.3.1 above using DELFIA enhancement solution.

Of the 1692 clones screened, 1029 clones yielded a signal of more than 2000 RFU in the DELFIA assay, giving a success rate of around 61%. The top 736 clones were then selected and analysed using a secondary assay (affinity ranking) employing three concentrations of hPD-L1-rCD4-His antigen (1.0 nM, 0.2 nM, and 0.04 nM). From the 736 clones screened, the 48 clones which showed the greatest signal were selected for cloning and expression in IgG1 format. Clones were expressed in Expi293F™ (Fisher Scientific cat. no. 13479756) cells at 800 μl scale, and the culture supernatants were harvested on the 5th day post transfection for further screening in IgG1 format.

### 2.2 SPR Screening

All 48 antibodies were ranked by affinity using SPR (Biacore T200 instrument). For ranking, diluted supernatants (1:10 in running buffer made of 1×PBS and 0.002% Tween™-20) were immobilised onto a Protein-A chip (GE healthcare, product code: 29127556) and human PD-L1-rCD4-His (SEQ ID NO: 79) was flowed over the prepared surface at 50 nM concentration. The association (kₐ) and dissociation (k_{d}) rate constants generated using this single injection were used to determine an estimated dissociation constant (K_{D}). The K_{D} values of the clones were compared with that of clone 280_02_G02_NS in Ig1 format (G1/280_02_G02_NS). Ten clones of unique sequence were identified that showed higher affinity for human PD-L1 than clone G1/280_02_G02_NS and were therefore subjected to full kinetic analysis together with clone G1/280_02_G02_NS.

Briefly, SPR experiments were performed using a BIAcore T200 instrument. Antibodies from diluted culture supernatant were captured on a Protein A chip (GE Healthcare, 29127556) over FC2 at a flow rate of 10 μl/min, with 60 seconds contact time. Typically, this resulted in 500-800 RU of antibody captured. Doubling dilutions of PD-L1-rCd4-His, ranging from 50 to 0.05 nM, were injected from 50 nM at a flow rate of 30 μl/min, (Concentration range: 50 nM-0.05 nM) over FC1 and FC2. Association was measured over 180 seconds, and dissociation was measured over 300 seconds. All measurements were performed at 25° C. in PBS, PH 7.4, 0.05% Tween™-20. Kinetic parameters were determined by reference cell subtraction and fitting the sensogram experimental data assuming a 1:1 interaction using the BIAevaluation software (GE, BR-1005-97). The resulting data was fitted using BIAevaluation software and corresponding kₐ, kₐ, and K_{D} values were calculated. Out of the ten clones tested, four antibodies, designated “G1/887_04_E12”, “894_08_A05”, “G1/894_08_E05” and “G1/887_04_G12”, exhibited sub-nanomolar K_{D} values, which were lower than the K_{D} for G1/280_02_G02_NS. The affinity data obtained under the described screening conditions showed that the kappa light chain shuffle described in Example 2 allowed the heavy chain of the G1/280_02_G02_NS antibody to be paired not only with a lambda light chain but also with kappa light chains to produce antibodies with good, and in fact improved, affinity for recombinant human PD-L1. Accurate affinities generated with low levels of captured mAb are reported in Table 3 Section 3.2.

### Example 3: Characterisation of Kappa Clones in IgG1 Format

### 3.1 Cell Based PD-1/PD-L1 Blocking Assay

The ability of the anti-PD-L1 clones containing a kappa light chain, G1/887_04_E12, G1/894_08_E05 and G1/887_04_G12, to block the interaction between PD1 and PD-L1 was assessed in a bioluminescent cell-based assay using a PD1/PD-L1 Blockade Bioassay product (Promega, J1250/J1255) in accordance with the manufacturer's recommendations. The blocking activity was compared to the G1/280_02_G02_NS clone.

Briefly, all antibodies were expressed and purified as described in section 1.3.3 and tested at 3-fold dilutions from 100 nM to 35 PM (eight concentrations) in duplicates. The calculated IC₅₀ values are shown in Table 3. All clones tested were shown to be potent inhibitors of the PD1/PD-L1 interaction, with the three kappa light chain-containing clones G1/887_04_E12, G1/894_08_E05 and G1/884_04_G12 exhibiting even better IC₅₀ values than the lambda light chain-containing clone G1/280_02_G02_NS.

TABLE 3

| Clone ID | IC₅₀ (nM) |
|---|---|
| G1/280_02_G02_NS | 0.51 |
| G1/887_04_E12 | 0.35 |
| G1/894_08_E05 | 0.36 |
| G1/887_04_G12 | 0.33 |

### 3.2 Affinities

The binding of the anti-PD-L1 mAbs G1/887_04_E12, G1/887_04_G12 and G1/894_08_E05 to recombinant human biotinylated human PD-L1-Avi-His (Acro Biosystems, PD1-H82E5), cynomolgus PD-L1-His (Acro Biosystems, PD1-C52H4) and mouse PD-L1-His (Acro Biosystems, PD1-M5220) was then measured by SPR using a Biacore T200 instrument (GE Healthcare). Affinities were compared to the 280_02_G02_NS clone in IgG1 format (G1AA/280_02_G02_NS; the “AA” in this clone name denotes that this clone also contained the “LALA” mutation in the CH2 domain).

Briefly, the anti-PD-L1 mAbs, diluted in HBS-EP buffer (GE Healthcare, BR100188) at 2 μg/ml, were injected individually on flows cell 2, 3 and 4 of a Protein A chip (GE Healthcare, 29127556) at 30 μl/min to achieve a final response of approximately 110 RU. The recombinant human, cynomolgus and mouse PD-L1-His antigens, diluted in HBS-EP buffer, were injected on flow cell 1, 2, 3 or 4 as appropriate at a concentration range of 81 nM to 0.037 nM with 3-fold dilutions for 4 min at 75 μl/min and then allowed to dissociate in buffer for 10 min. Regeneration was achieved by injecting 10 mM glycine-HCL pH1.5 (GE Healthcare, Human Antibody Capture Kit, BR00356) for 30 sec at a rate of 30 μl/min. Subtracted data (flow cell 2-flow cell 1, flow cell 3-flow cell 1, or flow cell 4-flow cell 1) were analysed using BIAevaluation 3.2 Software (GE Healthcare) to identify binding using the model 1:1 binding with mass transfer, with refractive index (RI) constant 0. To determine the affinities of the mouse PD-L1 binding curves, the Rmax of the corresponding human binding profiles was used.

The binding data demonstrated that the G1AA/280_02_G02_NS clone and the G1/894_08_E05, G1/887_04_E12 and G1/887_04_G12 clones bound to human and cynomolgus PD-L1 with low single-digit nanomolar or subnanomolar affinities and were fully human/cynomolgus cross-reactive (Table 4). In comparison to the G1AA/280_02_G02_NS clone, the binding affinities of the G1/894_08_E05, G1/887_04_E12 and G1/887_04_G12 clones were about 1.8 to 4.8 fold higher for human PD-L1 and 2.7 to 4.7 fold higher for cynomolgus PD-L1. The affinities of the clones for recombinant mouse PD-L1 were lower, with K_{D} values ranging from 38 to 225 nM, with the highest affinity being observed for the G1/887_04_E12 clone. These data show that the heavy chain of the G1AA/280_02_G02_NS antibody can be paired with both lambda and kappa light chains to produce antibodies with good (and in the case of kappa light chain pairing, sub-nanomolar) affinities for recombinant human and cynomolgus PD-L1, as well as some, albeit lower, affinity for recombinant mouse PD-L1.

TABLE 4

| | Human | Cynomolgus | Mouse |
|---|---|---|---|
| | PD-L1-His | PD-L1-His | PD-L1-His |
| Clone | K_{D} (nM) | K_{D} (nM) | K_{D} (nM) |
| G1AA/280_2_G02_NS | 1.10 | 1.32 | 137 |
| G1/894_08_E05 | 0.60 | 0.49 | 223 |
| G1/887_04_E12 | 0.23 | 0.28 | 38 |
| G1/887_04_G12 | 0.39 | 0.45 | 225 |

3.3 Binding of Anti-PD-L1 mAbs to Cell Expressed PD-L1

### 3.3.1 Generation of Cells Overexpressing Human PD-L1

To assess binding of the anti-PD-L1 mAbs G1/894_08_E05, G1/887_04_E12 and G1/887_04_G12 to cell surface PD-L1, HEK293 cells overexpressing human PD-L1 were generated.

Human PD-L1 sequence (SEQ ID NO: 83) was subcloned into pcDNA™5/FRT vector (ThermoFisher Scientific Cat. No. V601020) using KpnI and NotI restriction sites and the vector was then transformed into Flp-In T-REx 293 cell line (Life Technologies, R780-07) using Lipofectamine 2000 (Life Technologies, 11668-019). Cells were grown in DMEM containing 10% FBS, 100 μg/ml Hygromycin B (Melford Laboratories Ltd, Z2475) and 15 μg/ml Blasticidin (Melford Laboratories Ltd, B1105) for 3-4 weeks until colonies of stably transformed cells had formed. These colonies were amplified in the presence of 1 μg/ml doxycycline (Sigma Aldrich, D9891) and tested for expression of PD-L1 using PE-conjugated anti-human PD-L1 (MIH1) antibody (BD Biosciences, 557924). Cells were detached using cell dissociation buffer, washed once with PBS, plated at 2×10⁵ cells in wells of a 96-well plate and then incubated with antibody diluted 1:20 in PBS for 1 hour at 4° C., before being washed again in PBS and measured using an Accuri C6 cytometer (BD Biosciences). The data was analysed using FlowJoX software. Expression of human PD-L1 was detected in the cell line.

### 3.3.2 Cell Binding Assay: Cell Binding to HEK293-hPD-L1 and Control HEK-FRT Shows Specific Binding.

The anti-human PD-L1 mAbs, G1/894_08_E05, G1/887_04_E12 and G1/887_04_G12 were then tested for binding to HEK293 cells expressing human PD-L1 using flow cytometry. Non-specific binding was also assessed by testing binding to HEK293 parental cells lacking human PD-L1 (Flp-In T-Rex 293 cell line, Life Technologies, R780-07).

HEK293 and HEK293.hPD-L1 suspensions were prepared in PBS containing 0.5% BSA (Sigma. A7906) and seeded at 1×10⁵ cell/well in 100 μl in round bottomed 96-well plates (VWR, 734-1797). Cells were washed once in 100 μl 1×DPBS and mAbs G1/894_08_E05, G1/887_04_E12 and G1/887_04_G12 were diluted (1.10⁻⁶-0.013 nM, 5-fold dilutions) in 100 μl 1×DPBS (Gibco, 14190-094). The washed cells were resuspended in the diluted antibody mixture, incubated at 4° C. for 30 minutes, and then washed once in PBS. 100 μl/well of secondary antibody (Alexa Fluor 647-AffiniPure Goat Anti-Human IgG, F(ab′)2 Fragment Specific, Stratech Scientific, 109-605-006-JIR) diluted 1:1000 in PBS was then added, the cells/antibody mixture was incubated for 20 mins at 4° C., and the cells were then washed again with PBS and resuspended in 100 μl of PBS containing 7AAD (1:1000, Biotium, 40043) before being analysed using a Canto II flow cytometer (BD Bioscience). Dead cells were excluded and the fluorescence in the FITC channel (488 nm/530/30) was measured. The geometric mean fluorescence intensity (GMFI) values were plotted against the log concentration of antibody and the resulting curves were fitted using the log (agonist) versus response equation in GraphPad Prism.

The G1/894_08_E05, G1/887_04_E12 and G1/887_04_G12 clones were found to bind to cell surface human PD-L1 with EC₅₀ values in the range of 0.26-0.29 nM (see Table 5). No binding to parental HEK293 cells was observed showing the specificity of the binding. Therefore, all mAb clones tested bound specifically to PD-L1, with no non-specific binding observed.

TABLE 5

| | HEK293:hPD-L1 | HEK293-FRT negative |
|---|---|---|
| Clone | EC₅₀ (nM) | control cells |
| G1/894_08_E05 | 0.29 | No binding |
| G1/887_04_E12 | 0.26 | No binding |
| G1/887_04_G12 | 0.27 | No binding |

3.4 Activity of Anti-PD-L1 mAbs in Mixed Lymphocyte Reaction Assay

The activity of the anti-PD-L1 mAbs was tested in a Mixed Lymphocyte Reaction (MLR) assay. An MLR assay measures the cellular immune response that occurs between two allogeneic lymphocyte populations (same species but genetically distinct). The assay uses CD4+ T cells from one donor and monocyte derived dendritic cells (iDCs) from another donor. As the immune cells contain physiological levels of immune checkpoint regulators, the MLR assay can be used to confirm that T cell activation is enhanced by the mAb in a human system.

### 3.4.1 Generation of Expanded CD4+ T Cells

PBMCs were isolated from leukocyte cones by Ficoll gradient separation. CD4+ T cells were isolated using a Human CD4+ T Cell Isolation Kit (Miltenyi Biotec Ltd, 130-096-533) according to the manufacturer's instructions. Human T-Activator CD3/CD28 Dynabeads (Life Technologies, 11131D) were resuspended by vortexing. Beads were transferred to a sterile 15 ml tube and 10 ml RPMI (Life Technologies, 61870044) with 10% FBS (Life Technologies, 10270106) and 1× Penicillin Streptomycin (Life Technologies, 15140122) was added to wash the Dynabeads. The supernatant was discarded. The required amount of CD4+ T cells at 1.0×10⁶ cells/ml in RPMI with 10% FBS and 1× Penicillin Streptomycin Solution and 50 IU/ml recombinant human IL2 (Peprotech, 200-02-50 μg) with 3:1 bead to cell ratio was transferred to a T75 flask (Greiner Bio-one, 690195) and incubated at 37° C.+5% CO₂. After 3 days the cells were gently resuspended and counted. The cell density was maintained between 0.8-1×10⁶ cells/ml by adding fresh media (RPMI-10% FBS+ Penicillin Streptomycin Solution 1X+50 IU/ml rhuIL2) as needed. On day 7 or 8, the CD3/28 beads were removed and CD4+ T cells were rested overnight at 1×10⁶ cells/ml fresh media RPMI-10% FBS+Penicillin Streptomycin Solution 1X with reduced 10 IU/ml rhuIL2. The cells were stored frozen until required.

### 3.4.2 Generation of iDC

Untouched monocytes were isolated from human PBMCs using a Human Pan Monocyte Isolation Kit, (Miltenyi Biotec Ltd, 130-096-537) following the manufacturer's instructions. Monocytes were differentiated to iDCs using Human Mo-DC Differentiation Medium (Miltenyi Biotec Ltd, 130-094-812) following the manufacturer's instructions.

### 3.4.3 MLR Assay

Expanded T cells were thawed one day before the experiment, washed with AIM V Medium (Gibco, 12055-091) and incubated at 37° C., 5% CO₂ in AIM V Medium overnight. The anti-human PD-L1 mAbs, G1/894_08_E05, G1/887_04_E12 and G1/887_04_G12 were diluted at 4x the final concentration in triplicate in 50 μl AIM V Medium in 96 well round bottom plates (VWR, 734-1797). An anti-FITC antibody, designated 4420 (Bedzyk et al., 1989; Bedzyk et al., 1990), containing the LALA mutation was included as negative control. A 3-fold dilution series starting from 30 nM to 0.002 nM was tested. Both 1×104 iDC cells suspended in 50 μl AIM V Medium and 1×10⁵ expanded CD4+ T cells suspended in 100 μl AIM V Medium were added to the antibody dilutions and incubated for 5 days at 37° C.+5% CO₂. The following controls were included: CD4+ T cells alone, iDC alone, CD4+ T cells+iDCs, and AIM V Medium only. Supernatants were harvested, samples were diluted (1:25) and interferon gamma (IFN-Y) concentrations measured using Human IFN gamma ELISA Ready-SET-Go! Kit (Life Technologies, 88-7316-77). Plates were read at 450 nm using the plate reader with the Gen5 Software, BioTek. Absorbance values of 630 nm were subtracted from those of 450 nm (Correction). The standard curve for calculation of cytokine concentration was based on a four parameter logistic curve fit (Gen5 Software, BioTek). The concentration of human IFN-γ was plotted vs the log concentration of antibody and the resulting curves were fitted using the log (agonist) vs response equation in GraphPad Prism.

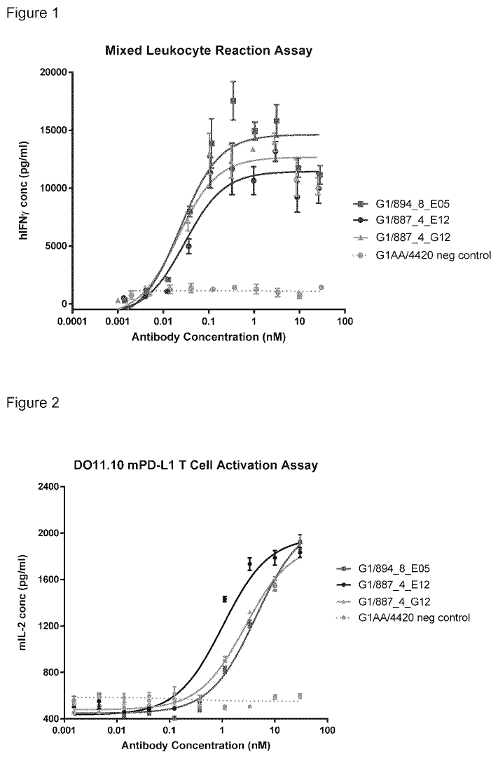

The anti-human PD-L1 mAbs, G1/894_08_E05, G1/887_04_E12 and G1/887_04_G12, showed potent activity in the MLR assay with EC₅₀ values of less than 0.030 nM and a maximum level of IFN-γ (Eₘₐₓ) of greater than 10000 μg/ml (Table 6, representative FIG. 1). The EC₅₀ indicates the concentration of mAb at which half of the response is achieved, whereas the Eₘₐₓ is an absolute value that indicates the maximum concentration of IFN-γ achieved in the assay. No activity was observed with the negative control G1AA/4420 mAb, as expected.

TABLE 6

| | Functional activity in MLR assay | |
|---|---|---|
| Clone | EC₅₀ (nM) | Eₘₐₓ (pg/ml) |
| G1/894_08_E05 | 0.024 | 14620 |
| G1/887_04_E12 | 0.029 | 11440 |
| G1/887_04_G12 | 0.021 | 12670 |

| | |
|---|---|
| G1AA/4420 negative | No activity |
| control | |

3.5 Activity of Anti-PD-L1 mAbs in a Mouse DO11.10 T Cell Activation Assay

As the anti-human PD-L1 mAbs G1/887_04_E12, G1/887_4_G12 and G1/894_08_E05 were shown to be weakly crossreactive to mouse PD-L1 (see Example 3.2, Table 4) their functional activity towards mouse PD-L1 was examined in an interleukin-2 (IL-2) release assay based on the DO11.10 OVA T-lymphocyte and LK35.2 B-lymphocyte hybridoma cell lines. IL-2 release is a marker of T cell activation. T cells expressing endogenous murine PD-1 were transfected with empty vector (pLVX). B-cells were transfected with a mouse PD-L1 construct.

3.5.1 Production of T Cell Lines with an Empty Vector

Lentiviral transduction methodology was used to generate DO11.10 cells (National Jewish Health) containing the empty lentiviral vector pLVX using the Lenti-X HTX Packaging System (Clontech, 631249). Lenti-X expression vector (pLVX) (Clontech, 631253) was co-transfected with a Lenti-X HTX Packaging Mix into the Lenti-X 293T Cell Line (Clontech, 632180) to generate virus. The DO11.10 cell line was transduced using the lentiviral particles produced with the Lenti-X HTX Packaging System.

### 3.5.2 Production of Antigen Presenting Cells Over-Expressing PD-L1

Lentiviral transduction methodology was used to generate LK35.2 B cell lymphoma cells (ATCC, HB-98) over-expressing mouse PD-L1 using the Lenti-X HTX Packaging System (Clontech, 631249). Lenti-X expression vector (pLVX) (Clontech, 631253) containing, mouse PD-L1 cDNA (encoding the mouse PD-L1 of SEQ ID NO: 84), was co-transfected with a Lenti-X HTX Packaging Mix into the Lenti-X 293T Cell Line (Clontech, 632180) to generate virus. The LK35.2 cell line was transduced using the lentiviral vectors produced with the Lenti-X HTX Packaging System.

### 3.5.3 Mouse DO11.10 T Cell Activation Assay

Dilutions of the anti-PD-L1 mAbs G1/887_04_E12, G1/887_04_G12 and G1/894_08_E05 or the anti-FITC negative control mAb (G1AA/4420) were prepared in experimental media (DMEM (Gibco, 61965-026), 10% FBS (Gibco, 10270-106), 1 mM Sodium Pyruvate (Gibco, 11360-070)). The mAbs were mixed 1:1 with 4×105/ml LK35.2 mPD-L1 cells in experimental media in presence of 2.46 μM OVA peptide (H-ISQAVHAAHAEINEAGR-OH, Pepscan) (100 μL LK35.2 mPD-L1 cells (B cell hybridoma transduced with a lentiviral vector containing mPD-L1 to overexpress mouse PD-L1)/mAb mix per well in 96-round bottom plate) and incubated at 37° C., 5% CO₂ for 1 hour. 2×105 DO11.10 pLVX cells (DO11.10 T cell hybridoma transduced with an empty lentiviral vector) per ml in 100 μl volume experimental media were added to 100 μl of the LK35.2 mPD-L1/(mAbs) mix. The cells were then mixed before being incubated at 37° C., 5% CO₂ for 24 hours. Superatants were collected and assayed with mouse IL-2 ELISA kit (eBioscience, 88-7024-88 or R&D systems, SM2000) following the manufacturer's instructions. Plates were read at 450 nm using the plate reader with the Gen5 Software, BioTek. Absorbance values of 570 nm were subtracted from those of 450 nm (Correction). The standard curve for calculation of cytokine concentration was based on a four parameter logistic curve fit (Gen5 Software, BioTek). The concentration of mouse IL-2 was plotted vs the log concentration of mAb and the resulting curves were fitted using the log (agonist) vs response equation in GraphPad Prism.

The results are shown in FIG. 2 and Table 7. The anti-human PD-L1 mAbs showed significant activity in the mouse T cell activation assay with potencies (EC₅₀) in the range of 1-4.4 nM. No activity was observed with the negative control mAb as expected. Of the three clones tested, G1/887_04_E12, which showed the highest affinity for recombinant mouse PD-L1 (see Table 4), was also the most potent clone in the T cell activation assay. The differences in potency were smaller than the measured affinities which is likely due to the high overexpression of mouse PD-L1 on the LK35.2 cells in this assay.

TABLE 7

| | Functional activity in mPD-L1 | |
|---|---|---|
| | DO11.10 T Cell Activation Assay | |
| Clone | EC₅₀ (nM) | Eₘₐₓ (pg/ml) |
| G1/894_08_E05 | 4.37 | 2112 |
| G1/887_04_E12 | 1.02 | 1966 |
| G1/887_04_G12 | 2.71 | 1912 |
| G1AA/4420 | No activity | |
| negative | | |
| control | | |

### 3.6 Pharmacokinetics in Naïve Mice

To investigate the in vivo pharmacokinetics (PK), the anti-PD-L1 mAbs G1/894_08_E05, G1/887_04_E12, and G1/887_04_G12 were tested in a research-grade PK study in which the mAbs were administered to non-tumour bearing mice and the concentrations in the blood serum were measured over time.

C57/BL6 mice (female 9-10 weeks old) were divided into 4 groups of 3 animals to receive a single dose of the test antibody administered intravenously. The animals were dosed once with the anti-PD-L1 mAbs at 8 mg/kg. Antibodies were administered intravenously (100 μl, tail vein) and then blood samples (20 μl, tail vein) were collected at 7 different time points, from 3 mice per time point. The time points were 0.5, 1, 6, 24, 48, 96 and 144 hours post-dosing. Blood was allowed to clot at room temperature for 2 hours, spun in a centrifuge at 2000 g for 20 min, then the serum was recovered and stored at −80° C. For the analysis, all the serum samples were thawed and analysed at the same time on a Gyrolab xPlore machine (Gyrolab Technologies) using the 200-3W-002-A program. Biotinylated goat anti-human IgG-heavy and light chain monkey-adsorbed (Cambridge Bioscience, A80-319B) was used as the capture reagent and goat anti-human IgG-AF647 (Cambridge Bioscience, 2040-31) as the detection reagent. The concentration of human IgG was measured in each serum sample and real drug concentration was calculated based on titration curves for each compound to eliminate potential differences in detection. Additional standard curves were performed to validate that binding to the capture or detection anti-human IgG mAb was not altered by the mAbs.

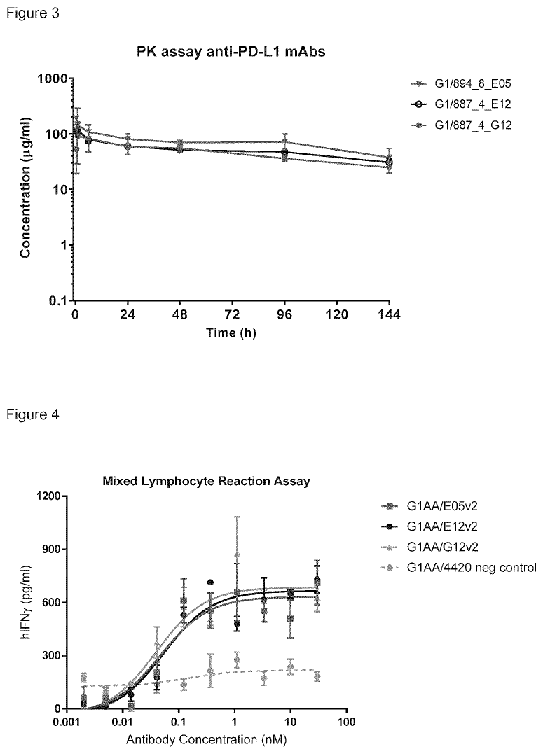

The anti-PD-L1 mAbs showed no initial rapid clearance and exposure levels were maintained at more than 24 μg/ml during the 6-day period (FIG. 3). This data is as expected for mAbs and in line with published anti-PD-L1 mAb data (Deng et al., 2016).

### 3.7 Summary of Characterisation of Kappa Clones in IgG1 Format

The anti-PD-L1 mAbs G1/894_08_E05, G1/887_04_E12, and G1/887_04_G12, containing the selected kappa light chains demonstrated cynomolgus and mouse PD-L1 cross-reactivity, showed specific binding to cell surface-expressed PD-L1, and showed even higher affinity for recombinant human and cynomolgus PD-L1 than the lambda light chain-containing clone G1/280_02_G02. The anti-PD-L1 mAbs G1/894_08_E05, G1/887_04_E12, and G1/887_04_G12 were shown to be potent activators of human T cells in vitro, to have functional mouse crossreactivity, and to have satisfactory PK profiles in non-tumour bearing mice.

### Example 4: Sequence Optimisation

### 4.1 Identification and Removal of Potential Protein Deamidation Sites

Analysis of the sequence of the G1/280_02_G02_NS clone resulted in the identification of the sequence NSNT (SEQ ID NO: 6) in the H-CDR2 loop (at Kabat positions 54-57) as a potential deamidation site, which, if deamidated, could affect binding. The heavy chain of this clone was retained in all kappa light chain-containing clones obtained by the chain shuffling campaign described in Example 2, so this potential deamidation site was also present in clones G1/887_04_E12, G1/894_08_A05, G1/894_08_E05 and G1/887_4_G12. Using specific primers closest to germline sequence, the NSNT (SEQ ID NO: 6) sequence was changed in the four kappa light chain-containing clones by site-directed mutagenesis to either GGST (SEQ ID NO: 7), SGGT (SEQ ID NO: 5) or SGNA (SEQ ID NO: 8) to produce the variant clones identified in Table 8. At the same time as removing this potential deamidation site, the role of the proline residue at Kabat position 28 in the VH region of the G1/887_04_E12 clone, which was unintentionally introduced into the sequence of this antibody during the kappa light chain shuffle, was also investigated by reverting it back to a threonine residue as contained in the G1/280_02_G02_NS clone. The parent and resulting variant clones (all in IgG1 format) were transfected at 0.8 ml scale, and culture supernatants harvested five days after transfections were used to determine the affinities of the clones for human and cynomolgus PD-L1-rCD4-His by SPR. Cyno PD-L1-rCD4-His was generated as described in Example 1.1. With the exception of the variant clones derived from the G1/887_04_E12 clone, all variant clones retained their sub-nanomolar affinities for human and cynomolgus PD-L1 as compared to their respective parent clone (see Table 8).

TABLE 8

| | H-CDR2 (position 54- | H-CDR1 | | |
|---|---|---|---|---|
| | 57) “NSNT” motif or | Proline | hPDL1 | cPDL1 |
| | germline (GGST, | (position | K_{D} | K_{D} |
| Clone ID | SGGT, or SGNA) | 28) | (nM) | (nM) |
| G1/280_02_G02_NS | − | − | 1.20 | 1.80 |
| G1/887_04_E12 | NSNT (SEQ ID NO: 6) | + | 0.21 | 0.25 |
| (Parent) | | | | |
| G1/929_01_A01 | GGST (SEQ ID NO: 7) | − | 2.66 | 3.18 |
| G1/929_01_A02 | SGGT (SEQ ID NO: 5) | − | 3.07 | 3.64 |
| G1/929_01_A03 | SGNA (SEQ ID NO: 8) | − | 2.66 | 3.31 |
| G1/894_08_A05 | NSNT (SEQ ID NO: 6) | − | 0.42 | 0.57 |
| (Parent) | | | | |
| G1/929_01_A04 | GGST (SEQ ID NO: 7) | − | 0.47 | 0.60 |
| G1/929_01_A05 | SGGT (SEQ ID NO: 5) | − | 0.55 | 0.62 |
| G1/929_01_A06 | SGNA (SEQ ID NO: 8) | − | 0.45 | 0.58 |
| G1/894_08_E05 | NSNT (SEQ ID NO: 6) | − | 0.48 | 0.59 |
| (Parent) | | | | |
| G1/929_01_A07 | GGST (SEQ ID NO: 7) | − | 0.47 | 0.59 |
| G1/929_01_A08 | SGGT (SEQ ID NO: 5) | − | 0.50 | 0.65 |
| G1/929_01_A09 | SGNA (SEQ ID NO: 8) | − | 0.49 | 0.57 |
| G1/887_04_G12 | NSNT (SEQ ID NO: 6) | − | 0.36 | 0.50 |
| (Parent) | | | | |
| G1/929_01_A10 | GGST (SEQ ID NO: 7) | − | 0.42 | 0.60 |
| G1/929_01_A11 | SGGT (SEQ ID NO: 5) | − | 0.51 | 0.57 |
| G1/929_01_A12 | SGNA (SEQ ID NO: 8) | − | 0.51 | 0.66 |

The much-reduced affinities of the G1/929_01_A01, G1/929_01_A02 and G1/929_01_A03 clones compared to their parent (G1/887_04_E12) were considered likely to be due to the removal of the proline in the VH region at Kabat position 28 rather than to the presence of the GGST (SEQ ID NO: 7), SGGT (SEQ ID NO: 5) and SGNA (SEQ ID NO: 8) substitutions in H-CDR2. It was surprising that this proline residue in the G1/887_04_E12 clone appeared to be important for its affinity for PD-L1. The variants derived from the three parent clones G1/887_04_E12, G1/894_08_E05 and G1/887_04_G12 which contained the SGGT (SEQ ID NO: 5) substitution in their H-CDR2 (positions 54-57), namely clones G1/929_01_A02, G1/929_01_A08 and G1/929_01_A11, were selected for further characterisation on the basis that this SGGT (SEQ ID NO: 5) substitution was closest to germline sequence.

Using site-directed mutagenesis, the potential deamidation site (NSNT (SEQ ID NO: 6) at Kabat position 54 to 57) in the H-CDR2 loop of the G1/280_02_G02_NS clone was also modified to SGGT (SEQ ID NO: 5). Additionally, a further potential deamidation site (NS motif) identified at Kabat positions 31 to 32 in the CDR1 of the lambda light chain of this clone was modified to NY by mutating serine 32 (Kabat numbering) to a tyrosine, as tyrosine is found at this position in several germline sequences, such as IGLV2-8-01, IGLV2-8-02, IGLV2-8-03, IGLV2-11-01, IGLV2-11-02, IGLV2-11-03 and IGLV2-14-01, IGLV2-14-02, IGLV2-14-03, IGLV2-14-04. The combination of these modifications yielded the lambda light chain-containing clone G1/lambdav3, which was also selected for further characterisation.

### Example 5: Characterisation of mAbs/mAb²

5.1 Cloning and Production of Clones in mAb and mAb² Format

The threonine residue at Kabat position 28 in the VH region of the G1/929_01_A02 “SGGT” variant clone identified in Example 4 was mutated to a proline, as is present at the same position in its parent clone G1/887_04_E12, with a view to improving its affinity for human and cynomolgus PD-L1. Transient expression in HEK293-6E cells and purification using mAb Select SuRe protein A columns was used to produce this modified variant clone and the other three “SGGT” variant clones (G1/929_01_A08, G1/929_01_A11 and G1/lambdav3) identified in Example 4 in IgG1 format and with the LALA mutation to enable testing of their functional activity in the absence of effector function. The resulting mAbs were designated G1AA/E12v2, G1AA/E05v2, G1AA/G12v2 and G1AA/lambdav3. The heavy and light chain sequences respectively are shown in SEQ ID NO: 47 and SEQ ID NO: 48 for G1AA/E12v2; SEQ ID NO: 49 and SEQ ID NO: 50 for G1AA/G12v2; SEQ ID NO: 51 and SEQ ID NO: 52 for G1AA/E05v2, and SEQ ID NO: 61 and SEQ ID NO: 62 for G1AA/lambdav3.

The CDR-based antigen-binding sites of a mAb can be combined with Fcab (fragment crystallisable antigen-binding) moieties generated in a constant domain to provide bispecific antibodies referred to as mAb².

The anti-PD-L1 antibodies of the invention were produced in anti-CD137/anti-PD-L1 mAb² format to test their specificity for human PD-L1. The mAb² were produced in IgG1 LALA format, with the heavy chain having an anti-human CD137 binding site in the CH3 domain of the Fcab moiety and a VH domain from anti-PD-L1 mAb clone G1AA/E12v2, G1AA/E05v2, G1AA/G12v2 or G1AA/lambdav3. To generate the mAb², the heavy chains were co-transfected with the corresponding light chain of the anti-PD-L1 mAbs. The mAb² were produced by transient expression in HEK293-6E cells and purified using mAb Select SuRe protein A columns to yield clones FS22-172-003AA/E12v2, FS22-172-003AA/G12v2, FS22-172-003AA/E05v2 and FS22-172-003AA/lambdav3. The heavy and light chain sequences respectively are shown in SEQ ID NO: 85 and SEQ ID NO: 86 for FS22-172-003AA/E12v2, SEQ ID NO: 87 and SEQ ID NO: 88 for FS22-172-003AA/G12v2, SEQ ID NO: 89 and SEQ ID NO: 90 for FS22-172-003AA/E05v2, and SEQ ID NO: 91 and SEQ ID NO: 92 for FS22-172-003AA/lambdav3.

### 5.2 Affinities of mAb for Human and Cynomolgus PD-L1

To determine whether the further sequence modifications present in G1AA/lambdav3 (namely, NSNT (SEQ ID NO: 6) to SGGT (SEQ ID NO: 5) in the VH-CDR2, NS to NY in the VL-CDR1, and the LALA mutation) and the kappa light chain-containing mAbs G1AA/E12v2, G1AA/E05v2, and G1AA/G12v2 (namely, the LALA mutation and, in G1AA/E12v2 only, threonine to proline at Kabat position 28 in the VH region) had affected binding kinetics, the affinities of these anti-PD-L1 mAbs for human and cynomolgus PD-L1 were determined as described in Example 3.2. The mAbs G1AA/lamdav3, G1AA/E05v2, G1AA/E12v2 and G1AA/G12v2 exhibited affinities for human and cynomolgus PD-L1 similar to those observed in Example 3.2 (Table 4) for mAbs G1AA/280_02_G02_NS, G1/894_08_E05, G1/887_04_E12 and G1/887_04_G12, demonstrating that the binding affinities of the mAbs and mAb² tested were not affected by the modification of the potential deamidation sites or the introduction of the LALA mutation. The G1AA/E12v2 mAb showed the lowest K_{D} value of all four mAbs tested (0.21 nM for human PD-L1, and 0.37 nM for cynomolgus PD-L1).

The VH of G1AA/E12v2 differs from that of G1/929_01_A02 (Example 4, Table 8) by one residue; G1AA/E 12v2 has a proline at Kabat position 28 whereas G1/929_01_A02 has a threonine at this position. G1/929_01_A02 had a greater than 10-fold lower affinity for both human and cynomolgus PD-L1 when compared to G1AA/E12v2; this data demonstrates the importance of the proline residue at position 28 (Kabat nomenclature) in the VH of clone G1AA/E12v2 for its affinity for human and cynomolgus PD-L1.

TABLE 9

| | Human PD-L1-His | Cyno PD-L1-His |
|---|---|---|
| mAb | K_{D} (nM) | K_{D} (nM) |
| G1AA/lambdav3 | 1.34 | 2.45 |
| G1AA/E05v2 | 0.50 | 0.89 |
| G1AA/E12v2 | 0.21 | 0.37 |
| G1AA/G12v2 | 0.44 | 0.75 |

### 5.3 Specificity for PD-L1 Family Members

PD-L1 belongs to the B7 homology family of immune checkpoint regulators (Ni and Dong, 2017). To analyse specificity of the anti-PD-L1 Fab arms of the mAb² clones FS22-172-003AA/lambdav3, FS22-172-003AA/E05v2, FS22-172-003AA/E12v2 and FS22-172-003AA/G12v2, their ability to bind to closely-related family members was tested using SPR. The aim was to demonstrate specificity by showing no binding of the mAb² to closely-related antigens at a concentration of 1 μM, but showing binding to PD-L1 receptors at a concentration of 1 nM.

Flow cells on CM5 chips were immobilised with approximately 1000 RU of either human PD-L2-Fc (R&D Biosystems, 1224-PL), CD80-Fc (R&D Biosystems, 140-B1), PD-1-His (R&D Biosystems, 8986-PD), B7-H3-His (F-star in-house production), PD-L1-Fc (R&D Biosystems, 156-B7) or PD-L1-His (Acrobiosystems, PD1-H83F3). Flow cell 1 was run as a blank immobilisation. The mAb² were diluted to 1 μM and 1 nM in 1x HBS-EP buffer (GE Healthcare, product code BR100188), allowed to flow over the chip for 3 min and then allowed to dissociate for 4 min. A 30-seconds injection of 10 mM glycine pH 1.5 was used for regeneration. Positive control mAbs were injected at 50-100 nM to demonstrate the coating of each antigen. Binding levels were determined at the end of the association phase and compared.

All the mAb² clones tested showed a high level of specificity, with less than 10 RU of mAb² binding to the four antigens detected at 1 μM compared to a range of 105 to 570 RU of binding response detected at 1 nM for binding to either human PD-L1-Fc or PD-L1-His. These results showed that specificity of the Fab arms for PD-L1 was retained regardless of the modifications made to the CDRs to remove potential deamidation sites, and that the introduction of the LALA mutation and producing the Fabs in mAb² format did not affect their binding to PD-L1.

5.4 Activity of Anti-Human PD-L1 mAbs in MLR

The anti-PD-L1 mAbs, G1AA/E05v2, G1AA/E12v2 and G1AA/G05v2 were tested in a Mixed Lymphocyte Reaction (MLR) assay as described in Example 3.4. G1AA/4420 was used as a negative control. The data are shown in Table 10 and FIG. 4. The mAbs G1AA/E05v2, G1AA/E12v2 and G1AA/G12v2 showed potent activity in the MLR assay with EC₅₀ values of less than 0.054 nM and a maximum level of IFN-γ (Emax) of greater than 600 μg/ml (Table 10, FIG. 4. The EC₅₀ and especially the Emax values were significantly different from those described in Example 3.4. This difference is believed to be due to donor variability, as the response depends on the allogenic reaction between T cells from one donor and the monocyte derived dendritic cells from another donor. The potency of the anti-human PD-L1 mAbs was consistent with the data described in Example 3.4, as was the ranking of the clones by order of potency. No activity was observed for the negative control G1AA/4420 mAb, as expected.

TABLE 10

| | Functional activity in MLR Assay | |
|---|---|---|
| Clone | EC₅₀ (nM) | Eₘₐₓ (pg/ml) |
| G1AA/E05v2 | 0.047 | 632 |
| G1AA/E12v2 | 0.054 | 666 |
| G1AA/G12v2 | 0.040 | 686 |
| G1AA/4420 negative | No activity | |
| control | | |

5.5 Expression, Purification and Analytical Characterisation of Anti-PD-L1 mAbs

The mAbs G1AA/E05v2, G1AA/E12v2 and G1AA/G12v2 were produced at lab-scale and characterised by the standard analytical methods of SEC and Differential Scanning calorimetry (DSC).

5.5.1 Lab Scale Expression and Purification of Anti-PD-L1 mAbs

DNA sequences encoding the mAbs G1AA/E05v2, G1AA/E12v2 and G1AA/G12v2 were transfected into HEK293 6E (National Research Council Canada) cells using PEIpro (Polyplus, France). After 5 days, cell culture fluids were harvested, and purified on MabSelect Protein-A pre-packed columns using AKTAxpress instrument (both GE Healthcare, Uppsala, Sweden). Equilibration of the columns was carried out in 50 mM Tris, 250 mM NaCl at pH 7.0 followed by loading with harvested cell culture fluid. The resin was washed using 50 mM Tris, 250 mM NaCl at pH 7.0 and this was followed by eluting the mAb using buffer at pH of less than 3.5.

### 5.5.2 Analysis by SE-UPLC

Post-purification SE-UPLC was performed within 24 hours of purification (material was stored at 4° C.) using an Acquity H-Class Bio UPLC (Waters Corp. UK) to measure the percentage of monomer. An Acquity UPLC BEH200 SEC 1.7 mm column (4.6×150 mm) was used, the mobile phase consisted of 250 mM sodium phosphate, 100 mM L-Arginine at pH 6.8. Quantification of monomer, low molecular and high molecular weight species was performed using Empower software (Waters Corp, UK).

### 5.5.3 Thermostability

The melting temperature (Tₘ) of G1AA/E05v2, G1AA/E12v2 and G1AA/G12v2 was measured using a Microcal VP-capillary differential scanning calorimeter (DSC). G1AA/lambdav3 was included to assess the difference between the kappa and lambda light chain-containing mAbs. Samples were measured in sample buffer at a concentration of 0.2 mg/ml. The scan rate was set at 60° C./hr and data were collected between 35° C. and 100° C. Data analysis was performed with Origin 7.0 software. As the DSC peaks of the Fab and CH3 were overlapping, one value was reported.

TABLE 11

| | Monomer purity | |
|---|---|---|
| mAb | post-Protein A % | Tm of Fab/CH3 |
| G1AA/E05v2 | 99.48 ± 0.01% | 80.4-82.8° C. |
| G1AA/E12v2 | 98.85 ± 0.07% | 81.4-84.1° C. |
| G1AA/G12v2 | 99.83 ± 0.11% | 78.1-81.3° C. |
| G1AA/lambdav3 | 99.75 ± 0.25% | 68.1° C. |

A summary of the results is shown in Table 11. The three mAbs: G1AA/E05v2, G1AA/E12v2 and G1AA/G12v2 showed favourable analytical characterisation parameters; monomer purity post-protein A was greater than 98% and the thermal stability of the Fab transition (Tm) was found to be at the higher end of transitions typically reported for IgG1, with G1AA/E12v2 appearing to be the most thermally stable (Fab/CH3 T_{M}=81.4-84.1° C.). The lambda light chain mAb, G1AA/lambdav3, had a lower Tm than the three kappa light chain-containing mAbs.

### REFERENCES

Bedzyk W D, Johnson L S, Riordan G S, Voss E W Jr. Comparison of variable region primary structures within an anti-fluorescein idiotype family. J. Biol. Chem. 264(3), 1565-69 (1989).
Bedzyk W D, Weidner K M, Denzin L K, Johnson L S, Hardman K D, Pantoliano M W, Asel E D, Voss E W Jr. Immunological and structural characterization of a high affinity anti-fluorescein single-chain antibody. J Biol Chem. 265(30), 18615-20 (1990).
Brown M H, Barclay A N. Expression of immunoglobulin and scavenger receptor superfamily domains as chimeric proteins with domains 3 and 4 of CD4 for ligand analysis. Protein Eng. 7(4), 515-21 (1994).
Bruhns P, Iannascoli B, England P, Mancardi D A, Fernandez N, Jorieux S, Daeron M. Specificity and affinity of human Fcγ receptors and their polymorphic variants for human IgG subclasses. Blood 113(16), 3716-25 (2009).
Chapple S D, Crofts A M, Shadbolt S P, McCafferty J, Dyson M R. Multiplexed expression and screening for recombinant protein production in mammalian cells. BMC Biotechnol. 6:49(2006).
Curran M A, Montalvo W, Yagita H, Allison J P. PD-1 and CTLA-4 combination blockade expands infiltrating T cells and reduces regulatory T and myeloid cells within B16 melanoma tumors. Proc. Natl. Acad. Sci. U.S.A. 107(9), 4275-80 (2010).
Deng R, Bumbaca D, Pastuskovas C V, Boswell C A, West D, Cowan K J, Chiu H, McBride J, Johnson C, Xin Y, Koeppen H, Leabman M and Iyer S. Preclinical pharmacokinetics, pharmacodynamics, tissue distribution, and tumor penetration of anti-PD-L1 monoclonal antibody, an immune checkpoint inhibitor. MAbs 8(3), 593-603 (2016).
Dyson M R, Zheng Y, Zhang C, Colwill K, Pershad K, Kay B K, Pawson T, McCafferty J: Mapping protein interactions by combining antibody affinity maturation and mass spectrometry. Anal. Biochem. 417(1), 25-35 (2011).
Grosso J, Inzunza D. Wu Q, Simon J, Singh P, Zhang X, Phillips T, Simmons P, Cogswell J. Programmed death-ligand 1 (PD-L1) expression in various tumor types. J. Immunother Cancer. 1(Suppl 1), 53 (2013).
Herbst R S, Soria J C, Kowanetz M, Fine G D, Hamid O, Gordon M S, Sosman J A, McDermott D F, Powderly J D, Gettinger S N, Kohrt H E, Horn L, Lawrence D P, Rost S, Leabman M, Xiao Y, Mokatrin A, Koeppen H, Hegde P S, Mellman I, Chen D S, Hodi F S. Predictive correlates of response to the anti-PD-L1 antibody MPDL3280A in cancer patients. Nature 515 (7528), 563-7 (2014).
Hezareh M, Hessell A J, Jensen R C, van de Winkel J G, Parren P W. Effector function activities of a panel of mutants of a broadly neutralizing antibody against human immunodeficiency virus type 1. J. Virol. 75 (24), 12161-8 (2001).
Hu S, Shively L, Raubitschek A, Sherman M, Williams L E, Wong J Y, Shively J E, Wu A M. Minibody: A novel engineered anti-carcinoembryonic antigen antibody fragment (single-chain Fv-CH3) which exhibits rapid, high-level targeting of xenografts. Cancer Res. 56(13), 3055-61 (1996).
Idusogie E E, Presta L G, Gazzano-Santoro H, Totpal K, Wong P Y, Ultsch M, Meng Y G, Mulkerrin MG. Mapping of the C1q binding site on rituxan, a chimeric antibody with a human IgG1 Fc. J. Immunol. 164 (8), 4178-84 (2000).
Iwai Y, Ishida M, Tanaka Y, Okazaki T, Honjo T, Minato N. Involvement of PD-L1 on tumor cells in the escape from host immune system and tumor immunotherapy by PD-L1 blockade. Proc. Natl. Acad. Sci. U.S.A. 99 (19), 12293-7 (2002).
Jain T, Sun T, Durand S, Hall A, Houston N R, Nett J H, Sharkey B, Bobrowicz B, Caffry I, Yu Y, Cao Y, Lynaugh H, Brown M, Baruah H, Gray L T, Krauland E M, Xu Y, Vasquez M and Wittrup KD. Biophysical properties of the clinical-stage antibody landscape. PNAS 114 (5), 944-949 (2017).
Kabat E A, Wu T T, Perry H M, Gottesman K S, Foeller C. Sequences of Proteins of Immunological Interest, 5th ed. NIH Publication No. 91-3242. Washington, D.C.: U.S. Department of Health and Human Services (1991).
Kontermann R E. Dual targeting strategies with bispecific antibodies. MAbs 4(2), 182-97 (2012).
Klein C, Schaefer W, Regula J T. The use of CrossMAb technology for the generation of bi- and multispecific antibodies. MAbs. 8(6), 1010-20 (2016).
Larkin J, Hodi F S. Wolchok J D. Combined nivolumab and ipilimumab or monotherapy in untreated melanoma. N Engl J Med. 373(13), 1270-1 (2015).
Lefranc M P, Pommié C, Kaas Q, Duprat E, Bosc N, Guiraudou D, Jean C, Ruiz M, Da Piedade I, Rouard M, Foulquier E, Thouvenin V, Lefranc G. IMGT unique numbering for immunoglobulin and T cell receptor constant domains and Ig superfamily C-like domains. Dev. Comp. Immunol. 29(3), 185-203 (2005).
Martin C D, Rojas G, Mitchell J N, Vincent K J, Wu J, McCafferty J, Schofield D J. A simple vector system to improve performance and utilisation of recombinant antibodies. BMC Biotechnol. 6, 46 (2006).
Ni L, Dong C. New B7 family checkpoints in human cancers. Mol. Cancer Ther. 16(7), 1203-11 (2017).
Powles T, Eder J P, Fine G D, Braiteh F S, Loriot Y, Cruz C, Bellmunt J, Burris H A, Petrylak D P, Teng S L, Shen X, Boyd Z, Hegde P S, Chen D S, Vogelzang N J. MPDL3280A (anti-PD-L1) treatment leads to clinical activity in metastatic bladder cancer. Nature 515(7528), 558-62 (2014).
Rao M, Valentini D, Dodoo E, Zumla A, Maeurer M. Anti-PD-1/PD-L1 therapy for infectious diseases: learning from the cancer paradigm. Int. J. Infect. Dis. 56, 221-228 (2017). Schofield D J, Pope A R, Clementel V, Buckell J, Chapple S Dj, Clarke K F, Conquer J S, Crofts A M, Crowther S R, Dyson M R, Flack G, Griffin G J, Hooks Y, Howat W J, Kolb-Kokocinski A, Kunze S, Martin C D, Maslen G L, Mitchell J N, O'Sullivan M, Perera R L, Roake W, Shadbolt S P, Vincent K J, Warford A, Wilson W E, Xie J, Young J L, McCafferty J. Application of phage display to high throughput antibody generation and characterization. Genome Biol. 8(11), R254 (2007).
Spiess C, Zhai Q, Carter P J. Alternative molecular formats and therapeutic applications for bispecific antibodies. Mol. Immunol. 67(2 Pt A), 95-106 (2015).
Studier F W. Protein production by auto-induction in high density shaking cultures. Protein Expr. Purif. 41 (1), 207-34 (2005).
Wang X, Mathieu M, Brezski R J. IgG Fc engineering to modulate antibody effector functions. Protein Cell. 9 (1), 63-73 (2018).
Wolchok J D, Kluger H, Callahan M K, Postow M A, Rizvi N A, Lesokhin A M, Segal N H, Ariyan C E, Gordon R A, Reed K, Burke M M, Caldwell A, et al. Nivolumab plus ipilimumab in advanced melanoma. N. Engl. J. Med. 369 (2), 122-33 (2013).
Wykes M N, Lewin S R. Immune checkpoint blockade in infectious diseases. Nat. Rev. Immunol. 18(2), 91-104 (2017).

### Sequence Listing Information

### Antibody Sequences

### Notes:

i. The complete heavy chain, variable domain is shown in italics, CDRs according to the Kabat scheme are shown in italics and underlined, CDRs according to the IMGT scheme are shown in bold italics, therefore any overlapping IMGT and Kabat CDR sequences are shown in bold, italics and underlined, and, where applicable, location of LALA mutation is shown in bold and underlined.
ii. Amino acid and cDNA sequences for complete heavy chain are provided without optional C-terminal lysine.
iii. Complete light chain, variable domain are shown in italics, CDRs according to the Kabat scheme are shown in italics and underlined, CDRs according to the IMGT scheme are shown in bold italics, therefore any overlapping IMGT and Kabat CDR sequences are shown in bold, italics and underlined.
iv. In the amino acid sequence of variable domains, CDRs according to the Kabat scheme are shown in italics and underlined, CDRs according to the IMGT scheme are shown in bold italics, therefore any overlapping IMGT and Kabat CDR sequences are shown in bold, italics and underlined.
v. CDR amino acid sequences according to both Kabat and IMGT schemes are provided.

Amino acid sequence of heavy and light chains,
variable domains and CDRs of G1/280_02_G02

| | |
|---|---|
| Heavy | EVQLVQSGAEVKRPGASVKVSCKASGYTFTSYGISWVRQA |
| chain AA | PGQGLEWMGWISAYNGNTNYAQKLQGRVTMTTDTSTSTAY |
| (without | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 65) | TQTYICNVNHKPSNTKVDKKVEPKSCDKTHTCPPCPAPEL |
| | LGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| Heavy | EVQLVQSGAEVKRPGASVKVSCKASGYTFTSYGISWVRQA |
| chain AA | PGQGLEWMGWISAYNGNTNYAQKLQGRVTMTTDTSTSTAY |
| (with | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 93) | TQTYICNVNHKPSNTKVDKKVEPKSCDKTHTCPPCPAPEA |
| | AGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| VH | EVQLVQSGAEVKRPGASVKVSCKASGYTFTSYGISWVRQA |
| domain | PGQGLEWMGWISAYNGNTNYAQKLQGRVTMTTDTSTSTAY |
| AA | MELRSLRSDDTAVYYCARDLFPTIFGVSYYYYWGQGTLVT |
| (SEQ ID | VSS |
| NO: 45) | |
| HCDR1 | GYTFTSYG |
| (AA) | |
| (IMGT) | |
| (SEQ ID | |
| NO: 94) | |
| HCDR1 | SYGIS |
| (AA) | |
| (Kabat) | |
| (SEQ ID | |
| NO: 1) | |
| HCDR2 | ISAYNGNT |
| (AA) | |
| (IMGT) | |
| (SEQ ID | |
| NO: 95) | |
| HCDR2 | WISAYNGNTNYAQKLQG |
| (AA) | |
| (Kabat) | |
| (SEQ ID | |
| NO: 78) | |
| HCDR3 | ARDLFPTIFGVSYYYY |
| (AA) | |
| (IMGT) | |
| (SEQ ID | |
| NO: 69) | |
| HCDR3 | DLFPTIFGVSYYYY |
| (AA) | |
| (Kabat) | |
| (SEQ ID | |
| NO: 3) | |
| Light | QSALTQPASVSGSPGQSITISCTGTSSDVGGYNSVSWYQQ |
| chain AA | FPGKAPKLMIFEVTNRPSGVSDRFSGSKSDNTASLTISGL |
| (SEQ ID | QAEDEAEYYCSSFKRGSTLVVFGGGTKLTVLGQPAAAPSV |
| NO: 66) | TLFPPSSEELQANKATLVCLISDFYPGAVTVAWKADSSPV |
| | KAGVETTTPSKQSNNKYAASSYLSLTPEQWKSHRSYSCQV |
| | THEGSTVEKTVAPTECS |
| VL | QSALTQPASVSGSPGQSITISCTGTSSDVGGYNSVSWYQQ |
| domain | FPGKAPKLMIFEVTNRPSGVSDRFSGSKSDNTASLTISGL |
| AA | QAEDEAEYYCSSFKRGSTLVVFGGGTKLTVL |
| (SEQ ID | |
| NO: 46) | |
| LCDR1 | SSDVGGYNS |
| (AA) | |
| (IMGT) | |
| (SEQ ID | |
| NO: 96) | |
| LCDR1 | TGTSSDVGGYNSVS |
| (AA) | |
| (Kabat) | |
| (SEQ ID | |
| NO: 26) | |
| LCDR2 | EVT |
| (AA) | |
| (IMGT) | |
| (SEQ ID | |
| NO: 77) | |
| LCDR2 | EVTNRPS |
| (AA) | |
| (Kabat) | |
| (SEQ ID | |
| NO: 13) | |
| LCDR3 | SSFKRGSTLVV |
| (AA) | |
| (IMGT) | |
| (SEQ ID | |
| NO: 14) | |
| LCDR3 | SSFKRGSTLVV |
| (AA) | |
| (Kabat) | |
| (SEQ ID | |
| NO: 14) | |

Amino acid sequence of heavy and light chains,
variable domains and CDRs of G1/280_02_G02_NS

| | |
|---|---|
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (without | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 63) | TQTYICNVNHKPSNTKVDKKVEPKSCDKTHTCPPCPAPEL |
| | LGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (with | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 97) | TQTYICNVNHKPSNTKVDKKVEPKSCDKTHTCPPCPAPEA |
| | AGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| VH | EVQLVQSGAEVKRPGASVKVSCKASGYTFTSYGISWVRQA |
| domain | PGQGLEWMGWISAYNSNTNYAQKLQGRVTMTTDTSTSTAY |
| AA | MELRSLRSDDTAVYYCARDLFPTIFGVSYYYYWGQGTLVT |
| (SEQ ID | VSS |
| NO: 43) | |
| HCDR1 | GYTFTSYG |
| (AA) | |
| (IMGT) | |
| (SEQ ID | |
| NO: 94) | |
| HCDR1 | SYGIS |
| (AA) | |
| (Kabat) | |
| (SEQ ID | |
| NO: 1) | |
| HCDR2 | ISAYNSNT |
| (AA) | |
| (IMGT) | |
| (SEQ ID | |
| NO: 98) | |
| HCDR2 | WISAYNSNTNYAQKLQG |
| (AA) | |
| Kabat) | |
| (SEQ ID | |
| NO: 23) | |
| HCDR3 | ARDLFPTIFGVSYYYY |
| (AA) | |
| (IMGT) | |
| (SEQ ID | |
| NO: 69) | |
| HCDR3 | DLFPTIFGVSYYYY |
| (AA) | |
| (Kabat) | |
| (SEQ ID | |
| NO: 3) | |
| Light | QSALTQPASVSGSPGQSITISCTGTSSDVGGYNSVSWYQQ |
| chain AA | FPGKAPKLMIFEVTNRPSGVSDRFSGSKSDNTASLTISGL |
| (SEQ ID | QAEDEAEYYCSSFKRGSTLVVFGGGTKLTVLGQPAAAPSV |
| NO: 64) | TLFPPSSEELQANKATLVCLISDFYPGAVTVAWKADSSPV |
| | KAGVETTTPSKQSNNKYAASSYLSLTPEQWKSHRSYSCQV |
| | THEGSTVEKTVAPTECS |
| VL | QSALTQPASVSGSPGQSITISCTGTSSDVGGYNSVSWYQQ |
| domain | FPGKAPKLMIFEVTNRPSGVSDRFSGSKSDNTASLTISGL |
| AA | QAEDEAEYYCSSFKRGSTLVVFGGGTKLTVL |
| (SEQ ID | |
| NO: 44) | |
| LCDR1 | SSDVGGYNS |
| (AA) | |
| (IMGT) | |
| (SEQ ID | |
| NO: 96) | |
| LCDR1 | TGTSSDVGGYNSVS |
| (AA) | |
| (Kabat) | |
| (SEQ ID | |
| NO: 26) | |
| LCDR2 | EVT |
| (AA) | |
| (IMGT) | |
| (SEQ ID | |
| NO: 77) | |
| LCDR2 | EVTNRPS |
| (AA) | |
| (Kabat) | |
| (SEQ ID | |
| NO: 13) | |
| LCDR3 | SSFKRGSTLVV |
| (AA) | |
| (IMGT) | |
| (SEQ ID | |
| NO: 14) | |
| LCDR3 | SSFKRGSTLVV |
| (AA) | |
| (Kabat) | |
| (SEQ ID | |
| NO: 14) | |

Amino acid sequence of heavy and light chains,
variable domains and CDRs of G1/894_08_E05

| | |
|---|---|
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (without | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 57) | TQTYICNVNHKPSNTKVDKKVEPKSCDKTHTCPPCPAPEL |
| | LGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (with | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 99) | TQTYICNVNHKPSNTKVDKKVEPKSCDKTHTCPPCPAPEA |
| | AGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| VH | EVQLVQSGAEVKRPGASVKVSCKASGYTFTSYGISWVRQA |
| domain | PGQGLEWMGWISAYNSNTNYAQKLQGRVTMTTDTSTSTAY |
| AA | MELRSLRSDDTAVYYCARDLFPTIFGVSYYYYWGQGTLVT |
| (SEQ ID | VSS |
| NO: 37) | |
| HCDR1 | GYTFTSYG |
| (AA) | |
| (IMGT) | |
| (SEQ ID | |
| NO: 94) | |
| HCDR1 | SYGIS |
| (AA) | |
| (Kabat) | |
| (SEQ ID | |
| NO: 1) | |
| HCDR2 | ISAYNSNT |
| (AA) | |
| (IMGT) | |
| (SEQ ID | |
| NO: 98) | |
| HCDR2 | WISAYNSNTNYAQKLQG |
| (AA) | |
| Kabat) | |
| (SEQ ID | |
| NO: 23) | |
| HCDR3 | ARDLFPTIFGVSYYYY |
| (AA) | |
| (IMGT) | |
| (SEQ ID | |
| NO: 69) | |
| HCDR3 | DLFPTIFGVSYYYY |
| (AA) | |
| (Kabat) | |
| (SEQ ID | |
| NO: 3) | |
| Light | DIQMTQSPSTLSASVGDRVTITCRASQSISGRLAWYQQKP |
| chain AA | GKAPNLLIYEASNLESGVPSRFSGSGSGTEFTLTISSLQP |
| (SEQ ID | EDFATYYCQQSYSTPRVTFGQGTKVEIKRTAAAAPSVFIF |
| NO: 58) | PPSDEQLKSGTASVVCLLNNFYPREAKVQWKVDNALQSGN |
| | SQESVTEQDSKDSTYSLSSTLTLSKADYEKHKLYACEVTH |
| | QGLSSPVTKSFNRGEC |
| VL | DIQMTQSPSTLSASVGDRVTITCRASQSISGRLAWYQQKP |
| domain | GKAPNLLIYEASNLESGVPSRFSGSGSGTEFTLTISSLQP |
| AA | EDFATYYCQQSYSTPRVTFGQGTKVEIK |
| (SEQ ID | |
| NO: 38) | |
| LCDR1 | QSISGR |
| (AA) | |
| (IMGT) | |
| (SEQ ID | |
| NO: 100) | |
| LCDR1 | RASQSISGRLA |
| (AA) | |
| (Kabat) | |
| (SEQ ID | |
| NO: 19) | |
| LCDR2 | EAS |
| (AA) | |
| (IMGT) | |
| (SEQ ID | |
| NO: 71) | |
| LCDR2 | EASNLES |
| (AA) | |
| (Kabat) | |
| (SEQ ID | |
| NO: 20) | |
| LCDR3 | QQSYSTPRVT |
| (AA) | |
| (IMGT) | |
| (SEQ ID | |
| NO: 22) | |
| LCDR3 | QQSYSTPRVT |
| (AA) | |
| (Kabat) | |
| (SEQ ID | |
| NO: 22) | |

Amino acid sequence of heavy and light chains,
variable domains and CDRs of G1/887_04_E12

| | |
|---|---|
| Heavy | EVQLVQSGAEVKRPGASVKVSCKASGYPFTSYGISWVRQA |
| chain AA | PGQGLEWMGWISAYNSNTNYAQKLQGRVTMTTDTSTSTAY |
| (without | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 53) | TQTYICNVNHKPSNTKVDKKVEPKSCDKTHTCPPCPAPEL |
| | LGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| Heavy | EVQLVQSGAEVKRPGASVKVSCKASGYPFTSYGISWVRQA |
| chain AA | PGQGLEWMGWISAYNSNTNYAQKLQGRVTMTTDTSTSTAY |
| (with | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 101) | TQTYICNVNHKPSNTKVDKKVEPKSCDKTHTCPPCPAPEA |
| | AGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| VH | EVQLVQSGAEVKRPGASVKVSCKASGYPFTSYGISWVRQA |
| domain | PGQGLEWMGWISAYNSNTNYAQKLQGRVTMTTDTSTSTAY |
| AA | MELRSLRSDDTAVYYCARDLFPTIFGVSYYYYWGQGTLVT |
| (SEQ ID | VSS |
| NO: 33) | |
| HCDR1 | GYPFTSYG |
| (AA) | |
| (IMGT) | |
| (SEQ ID | |
| NO: 102) | |
| HCDR1 | SYGIS |
| (AA) | |
| (Kabat) | |
| (SEQ ID | |
| NO: 1) | |
| HCDR2 | ISAYNSNT |
| (AA) | |
| (IMGT) | |
| (SEQ ID | |
| NO: 98) | |
| HCDR2 | WISAYNSNTNYAQKLQG |
| (AA) | |
| (Kabat) | |
| (SEQ ID | |
| NO: 23) | |
| HCDR3 | ARDLFPTIFGVSYYYY |
| (AA) | |
| (IMGT) | |
| (SEQ ID | |
| NO: 69) | |
| HCDR3 | DLFPTIFGVSYYYY |
| (AA) | |
| (Kabat) | |
| (SEQ ID | |
| NO: 3) | |
| Light | DIQMTQSPSTLSASVRDRVIITCRASQSIGNRLAWYQHKP |
| chain AA | GKAPKLLIYEASTSETGVPSRFSGSGSGTDFTLTISSLQP |
| (SEQ ID | EDFATYYCQQSYSTPYTFGQGTKLEIKRTAAAAPSVFIFP |
| NO: 54) | PSDEQLKSGTASVVCLLNNFYPREAKVQWKVDNALQSGNS |
| | QESVTEQDSKDSTYSLSSTLTLSKADYEKHKLYACEVTHQ |
| | GLSSPVTKSFNRGEC |
| VL | DIQMTQSPSTLSASVRDRVIITCRASQSIGNRLAWYQHKP |
| domain | GKAPKLLIYEASTSETGVPSRFSGSGSGTDFTLTISSLQP |
| AA | EDFATYYCQQSYSTPYTFGQGTKLEIK |
| (SEQ ID | |
| NO: 34) | |
| LCDR1 | QSIGNR |
| (AA) | |
| (IMGT) | |
| (SEQ ID | |
| NO: 103) | |
| LCDR1 | RASQSIGNRLA |
| (AA) | |
| (Kabat) | |
| (SEQ ID | |
| NO: 15) | |
| LCDR2 | EAS |
| (AA) | |
| (IMGT) | |
| (SEQ ID | |
| NO: 71) | |
| LCDR2 | EASTSET |
| (AA) | |
| (Kabat) | |
| (SEQ ID | |
| NO: 16) | |
| LCDR3 | QQSYSTPYT |
| (AA) | |
| (IMGT) | |
| (SEQ ID | |
| NO: 17) | |
| LCDR3 | QQSYSTPYT |
| (AA) | |
| (Kabat) | |
| (SEQ ID | |
| NO: 17) | |

Amino acid sequence of heavy and light chains,
variable domains and CDRs of G1/887_04_G12

| | |
|---|---|
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (without | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 55) | TQTYICNVNHKPSNTKVDKKVEPKSCDKTHTCPPCPAPEL |
| | LGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (with | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 104) | TQTYICNVNHKPSNTKVDKKVEPKSCDKTHTCPPCPAPEA |
| | AGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| VH | EVQLVQSGAEVKRPGASVKVSCKASGYTFTSYGISWVRQA |
| domain | PGQGLEWMGWISAYNSNTNYAQKLQGRVTMTTDTSTSTAY |
| AA | MELRSLRSDDTAVYYCARDLFPTIFGVSYYYYWGQGTLVT |
| (SEQ ID | VSS |
| NO: 35) | |
| HCDR1 | GYTFTSYG |
| (AA) | |
| (IMGT) | |
| (SEQ ID | |
| NO: 94) | |
| HCDR1 | SYGIS |
| (AA) | |
| (Kabat) | |
| (SEQ ID | |
| NO: 1) | |
| HCDR2 | ISAYNSNT |
| (AA) | |
| (IMGT) | |
| (SEQ ID | |
| NO: 98) | |
| HCDR2 | WISAYNSNTNYAQKLQG |
| (AA) | |
| Kabat) | |
| (SEQ ID | |
| NO: 23) | |
| HCDR3 | ARDLFPTIFGVSYYYY |
| (AA) | |
| (IMGT) | |
| (SEQ ID | |
| NO: 69) | |
| HCDR3 | DLFPTIFGVSYYYY |
| (AA) | |
| (Kabat) | |
| (SEQ ID | |
| NO: 3) | |
| Light | DIQMTQSPSTLSASVGDRVTITCRASQSISGRLAWYQQKP |
| chain AA | GKAPNLLIYEASNLESGVPSRFSGSGSGTEFTLTISSLQP |
| (SEQ ID | EDFATYYCQQSYSWPRTFGQGTKVEIKRTAAAAPSVFIFP |
| NO: 56) | PSDEQLKSGTASVVCLLNNFYPREAKVQWKVDNALQSGNS |
| | QESVTEQDSKDSTYSLSSTLTLSKADYEKHKLYACEVTHQ |
| | GLSSPVTKSFNRGEC |
| VL | DIQMTQSPSTLSASVGDRVTITCRASQSISGRLAWYQQKP |
| domain | GKAPNLLIYEASNLESGVPSRFSGSGSGTEFTLTISSLQP |
| AA | EDFATYYCQQSYSWPRTFGQGTKVEIK |
| (SEQ ID | |
| NO: 36) | |
| LCDR1 | QSISGR |
| (AA) | |
| (IMGT) | |
| (SEQ ID | |
| NO: 100) | |
| LCDR1 | RASQSISGRLA |
| (AA) | |
| (Kabat) | |
| (SEQ ID | |
| NO: 19) | |
| LCDR2 | EASN |
| (AA) | |
| (IMGT) | |
| (SEQ ID | |
| NO: 77) | |
| LCDR2 | EASNLES |
| (AA) | |
| (Kabat) | |
| (SEQ ID | |
| NO: 20) | |
| LCDR3 | QQSYSWPRT |
| (AA) | |
| (IMGT) | |
| (SEQ ID | |
| NO: 21) | |
| LCDR3 | QQSYSWPRT |
| (AA) | |
| (Kabat) | |
| (SEQ ID | |
| NO: 21) | |

Amino acid sequence of heavy and light chains,
variable domains and CDRs of G1/894_08_A05

| | |
|---|---|
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (without | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 59) | TQTYICNVNHKPSNTKVDKKVEPKSCDKTHTCPPCPAPEL |
| | LGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| Heavy | EVQLVQSGAEVKRPGASVKVSCKASGYTFTSYGISWVRQA |
| chain AA | PGQGLEWMGWISAYNSNTNYAQKLQGRVTMTTDTSTSTAY |
| (with | MELRSLRSDDTAVYYCARDLFPTIFGVSYYYYWGQGTLVT |
| LALA) | VSSASTKGPSVFPLAPSSKSTSGGTAALGCLVKDYFPEPV |
| (SEQ ID | TVSWNSGALTSGVHTFPAVLQSSGLYSLSSVVTVPSSSLG |
| NO: 105) | TQTYICNVNHKPSNTKVDKKVEPKSCDKTHTCPPCPAPEA |
| | AGGPSVFLFPPKPKDTLMISRTPEVTCVVVDVSHEDPEVK |
| | FNWYVDGVEVHNAKTKPREEQYNSTYRVVSVLTVLHQDWL |
| | NGKEYKCKVSNKALPAPIEKTISKAKGQPREPQVYTLPPS |
| | RDELTKNQVSLTCLVKGFYPSDIAVEWESNGQPENNYKTT |
| | PPVLDSDGSFFLYSKLTVDKSRWQQGNVFSCSVMHEALHN |
| | HYTQKSLSLSPG |
| VH | EVQLVQSGAEVKRPGASVKVSCKASGYTFTSYGISWVRQA |
| domain | PGQGLEWMGWISAYNSNTNYAQKLQGRVTMTTDTSTSTAY |
| AA | MELRSLRSDDTAVYYCARDLFPTIFGVSYYYYWGQGTLVT |
| (SEQ ID | VSS |
| NO: 39) | |
| HCDR1 | GYTFTSYG |
| (AA) | |
| (IMGT) | |
| (SEQ ID | |
| NO: 94) | |
| HCDR1 | SYGIS |
| (AA) | |
| (Kabat) | |
| (SEQ ID | |
| NO: 1) | |
| HCDR2 | ISAYNSNT |
| (AA) | |
| (IMGT) | |
| (SEQ ID | |
| NO: 98) | |
| HCDR2 | WISAYNSNTNYAQKLQG |
| (AA) | |
| Kabat) | |
| (SEQ ID | |
| NO: 23) | |
| HCDR3 | ARDLFPTIFGVSYYYY |
| (AA) | |
| (IMGT) | |
| (SEQ ID | |
| NO: 69) | |
| HCDR3 | DLFPTIFGVSYYYY |
| (AA) | |
| (Kabat) | |
| (SEQ ID | |
| NO: 3) | |
| Light | DIQMTQSPSTLSASVGDRVTITCRASQSISGRLAWYQQKP |
| chain AA | GKAPNLLIYEASNLESGVPSRFSGSGSGTEFTLTINSLQP |
| (SEQ ID | DDFATYYCQQANTFPRVSFGGGTKVEIKRTAAAAPSVFIF |
| NO: 60) | PPSDEQLKSGTASVVCLLNNFYPREAKVQWKVDNALQSGN |
| | SQESVTEQDSKDSTYSLSSTLTLSKADYEKHKLYACEVTH |
| | QGLSSPVTKSFNRGEC |
| VL | DIQMTQSPSTLSASVGDRVTITCRASQSISGRLAWYQQKP |
| domain | GKAPNLLIYEASNLESGVPSRFSGSGSGTEFTLTINSLQP |
| AA | DDFATYYCQQANTFPRVSFGGGTKVEIK |
| (SEQ ID | |
| NO: 40) | |
| LCDR1 | QSISGR |
| (AA) | |
| (IMGT) | |
| (SEQ ID | |
| NO: 100) | |
| LCDR1 | RASQSISGRLA |
| (AA) | |
| (Kabat) | |
| (SEQ ID | |
| NO: 19) | |
| LCDR2 | EASN |
| (AA) | |
| (IMGT) | |
| (SEQ ID | |
| NO: 106) | |
| LCDR2 | EASNLES |
| (AA) | |
| Kabat) | |
| (SEQ ID | |
| NO: 20) | |
| LCDR3 | QQANTFPRVS |
| (AA) | |
| (IMGT) | |
| (SEQ ID | |
| NO: 24) | |
| LCDR3 | QQANTFPRVS |
| (AA) | |
| (Kabat) | |
| (SEQ ID | |
| NO: 24) | |

Amino acid and cDNA sequences of heavy and light chains and
variable domains of G1AA/E05v2 and amino acid sequence of CDRs

| | |
|---|---|
| Heavy chain AA | EVQLVQSGAEVKRPGASVKVSCKASGYTFTSYGISWVRQAPGQG |
| (without LALA) | LEWMGWISAYSGGTNYAQKLQGRVTMTTDTSTSTAYMELRSLRS |
| (SEQ ID NO: 107) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (without LALA) | GGAGCGTCCGTGAAGGTGTCCTGCAAAGCCTCAGGATACACCT |
| (SEQ ID NO: 108) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAACTGCTGGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 51) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (with LALA) | GGAGCGTCCGTGAAGGTGTCCTGCAAAGCCTCAGGATACACCT |
| (SEQ ID NO: 109) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAAGCTGCCGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| VH domain AA | EVQLVQSGAEVKRPGASVKVSCKASGYTFTSYGISWVRQAPGQG |
| (SEQ ID NO: 31) | LEWMGWISAYSGGTNYAQKLQGRVTMTTDTSTSTAYMELRSLRS |
| | DDTAVYYCARDLFPTIFGVSYYYYWGQGTLVTVSS |
| VH domain DNA | GAAGTGCAGCTGGTGCAGTCCGGAGCCGAAGTCAAGAGGCCT |
| (SEQ ID NO: 110) | GGAGCGTCCGTGAAGGTGTCCTGCAAAGCCTCAGGATACACCT |
| | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGC |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) (Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 52) | LIYEASNLESGVPSRFSGSGSGTEFTLTISSLQPEDFATYYCQQSY |
| | STPRVTFGQGTKVEIKRTVAAPSVFIFPPSDEQLKSGTASVVCLLNN |
| | FYPREAKVQWKVDNALQSGNSQESVTEQDSKDSTYSLSSTLTLSK |
| | ADYEKHKVYACEVTHQGLSSPVTKSFNRGEC |
| Light chain DNA | GACATTCAGATGACCCAATCCCCGTCCACGCTGAGCGCCTCCG |
| (SEQ ID NO: 112) | TCGGTGATCGCGTGACAATCACTTGTCGGGCGTCGCAGTCCAT |
| | CTCTGGAAGGCTCGCCTGGTACCAGCAGAAGCCTGGAAAGGCT |
| | CCCAACCTCCTTATCTACGAAGCCAGCAACCTGGAGTCCGGAG |
| | TGCCTAGCCGGTTCAGCGGATCAGGGTCCGGTACCGAGTTCAC |
| | CCTGACCATTTCCTCGCTCCAACCTGAGGACTTCGCCACCTACT |
| | ACTGCCAACAGTCCTATTCAACTCCGCGCGTGACCTTCGGCCA |
| | GGGCACTAAGGTCGAAATCAAAAGAACCGTGGCAGCCCCATCG |
| | GTGTTTATCTTCCCGCCCTCGGACGAACAGCTGAAGTCAGGCA |
| | CTGCTAGCGTGGTCTGTCTCCTGAACAATTTCTACCCGCGCGAA |
| | GCTAAGGTCCAGTGGAAGGTCGACAACGCGCTGCAGTCCGGAA |
| | ACAGCCAGGAGTCAGTGACCGAGCAGGACTCCAAGGATTCCAC |
| | TTATTCCCTGTCCTCCACCCTGACTTTGAGCAAGGCCGACTACG |
| | AGAAGCACAAAGTGTACGCCTGCGAAGTGACCCATCAAGGGCT |
| | TTCGTCGCCCGTGACCAAGAGCTTCAACCGGGGCGAATGC |
| VL domain AA | DIQMTQSPSTLSASVGDRVTITCRASQSISGRLAWYQQKPGKAPNL |
| (SEQ ID NO: 32) | LIYEASNLESGVPSRFSGSGSGTEFTLTISSLQPEDFATYYCQQSY |
| | STPRVTFGQGTKVEIK |
| VL domain DNA | GACATTCAGATGACCCAATCCCCGTCCACGCTGAGCGCCTCCG |
| (SEQ ID NO: 113) | TCGGTGATCGCGTGACAATCACTTGTCGGGCGTCGCAGTCCAT |
| | CTCTGGAAGGCTCGCCTGGTACCAGCAGAAGCCTGGAAAGGCT |
| | CCCAACCTCCTTATCTACGAAGCCAGCAACCTGGAGTCCGGAG |
| | TGCCTAGCCGGTTCAGCGGATCAGGGTCCGGTACCGAGTTCAC |
| | CCTGACCATTTCCTCGCTCCAACCTGAGGACTTCGCCACCTACT |
| | ACTGCCAACAGTCCTATTCAACTCCGCGCGTGACCTTCGGCCA |
| | GGGCACTAAGGTCGAAATCAAA |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EAS |
| (SEQ ID NO: 71) | |
| LCDR2 (AA) (Kabat) | EASNLES |
| (SEQ ID NO: 20) | |
| LCDR3 (AA) (IMGT) | QQSYSTPRVT |
| (SEQ ID NO: 22) | |
| LCDR3 (AA) (Kabat) | QQSYSTPRVT |
| (SEQ ID NO: 22) | |

Amino acid and cDNA sequences of heavy and light chains and
variable domains of G1AA/E12v2 and amino acid sequence of CDRs

| | |
|---|---|
| Heavy chain AA | EVQLVQSGAEVKRPGASVKVSCKASGYPFTSYGISWVRQAPGQG |
| (without LALA) | LEWMGWISAYSGGTNYAQKLQGRVTMTTDTSTSTAYMELRSLRS |
| (SEQ ID NO: 114) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (without LALA) | GGAGCGTCCGTGAAGGTGTCCTGCAAAGCCTCAGGATACCCCT |
| (SEQ ID NO: 115) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAACTGCTGGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| Heavy chain AA | EVQLVQSGAEVKRPGASVKVSCKASGYPFTSYGISWVRQAPGQG |
| (with LALA) | LEWMGWISAYSGGTNYAQKLQGRVTMTTDTSTSTAYMELRSLRS |
| (SEQ ID NO: 47) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (with LALA) | GGAGCGTCCGTGAAGGTGTCCTGCAAAGCCTCAGGATACCCCT |
| (SEQ ID NO: 116) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAAGCTGCCGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| VH domain AA | EVQLVQSGAEVKRPGASVKVSCKASGYPFTSYGISWVRQAPGQG |
| (SEQ ID NO: 27) | LEWMGWISAYSGGTNYAQKLQGRVTMTTDTSTSTAYMELRSLRS |
| | DDTAVYYCARDLFPTIFGVSYYYYWGQGTLVTVSS |
| VH domain DNA | GAAGTGCAGCTGGTGCAGTCCGGAGCCGAAGTCAAGAGGCCT |
| (SEQ ID NO: 117) | GGAGCGTCCGTGAAGGTGTCCTGCAAAGCCTCAGGATACCCCT |
| | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGC |
| HCDR1 (AA) (IMGT) | GYPFTSYG |
| (SEQ ID NO: 102) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) (Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVRDRVIITCRASQSIGNRLAWYQHKPGKAPKL |
| (SEQ ID NO: 48) | LIYEASTSETGVPSRFSGSGSGTDFTLTISSLQPEDFATYYCQQSY |
| | STPYTFGQGTKLEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |
| Light chain DNA | GACATCCAGATGACGCAGAGCCCGTCTACCCTGTCCGCCTCCG |
| (SEQ ID NO: 118) | TGAGAGATCGCGTGATCATCACCTGTCGGGCCAGCCAGTCCAT |
| | CGGAAACCGCTTGGCGTGGTACCAGCACAAGCCTGGGAAGGCT |
| | CCGAAGCTGCTCATCTACGAAGCCTCGACTTCGGAGACTGGTG |
| | TCCCTAGCCGGTTCAGCGGATCGGGATCAGGGACCGATTTCAC |
| | TCTGACCATTTCCTCCCTGCAACCCGAGGACTTCGCCACCTACT |
| | ACTGCCAACAGTCATATTCCACCCCGTACACCTTCGGACAAGGC |
| | ACCAAGCTCGAAATCAAGCGGACTGTCGCCGCACCTTCCGTGT |
| | TCATTTTCCCACCCTCCGACGAACAGCTGAAATCGGGTACAGCT |
| | AGCGTGGTCTGTCTCCTGAACAATTTCTACCCGCGCGAAGCTAA |
| | GGTCCAGTGGAAGGTCGACAACGCGCTGCAGTCCGGAAACAGC |
| | CAGGAGTCAGTGACCGAGCAGGACTCCAAGGATTCCACTTATTC |
| | CCTGTCCTCCACCCTGACTTTGAGCAAGGCCGACTACGAGAAG |
| | CACAAAGTGTACGCCTGCGAAGTGACCCATCAAGGGCTTTCGT |
| | CGCCCGTGACCAAGAGCTTCAACCGGGGCGAATGC |
| VL domain AA | DIQMTQSPSTLSASVRDRVIITCRASQSIGNRLAWYQHKPGKAPKL |
| (SEQ ID NO: 28) | LIYEASTSETGVPSRFSGSGSGTDFTLTISSLQPEDFATYYCQQSY |
| | STPYTFGQGTKLEIK |
| VL domain DNA | GACATCCAGATGACGCAGAGCCCGTCTACCCTGTCCGCCTCCG |
| (SEQ ID NO: 119) | TGAGAGATCGCGTGATCATCACCTGTCGGGCCAGCCAGTCCAT |
| | CGGAAACCGCTTGGCGTGGTACCAGCACAAGCCTGGGAAGGCT |
| | CCGAAGCTGCTCATCTACGAAGCCTCGACTTCGGAGACTGGTG |
| | TCCCTAGCCGGTTCAGCGGATCGGGATCAGGGACCGATTTCAC |
| | TCTGACCATTTCCTCCCTGCAACCCGAGGACTTCGCCACCTACT |
| | ACTGCCAACAGTCATATTCCACCCCGTACACCTTCGGACAAGGC |
| | ACCAAGCTCGAAATCAAG |
| LCDR1 (AA) (IMGT) | QSIGNR |
| (SEQ ID NO: 103) | |
| LCDR1 (AA) (Kabat) | RASQSIGNRLA |
| (SEQ ID NO: 15) | |
| LCDR2 (AA) (IMGT) | EAS |
| (SEQ ID NO: 71) | |
| LCDR2 (AA) (Kabat) | EASTSET |
| (SEQ ID NO: 16) | |
| LCDR3 (AA) (IMGT) | QQSYSTPYT |
| (SEQ ID NO: 17) | |
| LCDR3 (AA) (Kabat) | QQSYSTPYT |
| (SEQ ID NO: 17) | |

Amino acid and cDNA sequences of heavy and light chains and
variable domains of G1AA/G12v2 and amino acid sequence of CDRs

| | |
|---|---|
| Heavy chain AA | EVQLVQSGAEVKRPGASVKVSCKASGYTFTSYGISWVRQAPGQG |
| (without LALA) | LEWMGWISAYSGGTNYAQKLQGRVTMTTDTSTSTAYMELRSLRS |
| (SEQ ID NO: 120) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (without LALA) | GGAGCGTCCGTGAAGGTGTCCTGCAAAGCCTCAGGATACACCT |
| (SEQ ID NO: 121) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAACTGCTGGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 49) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (with LALA) | GGAGCGTCCGTGAAGGTGTCCTGCAAAGCCTCAGGATACACCT |
| (SEQ ID NO: 122) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAAGCTGCCGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| VH domain AA | EVQLVQSGAEVKRPGASVKVSCKASGYTFTSYGISWVRQAPGQG |
| (SEQ ID NO: 29) | LEWMGWISAYSGGTNYAQKLQGRVTMTTDTSTSTAYMELRSLRS |
| | DDTAVYYCARDLFPTIFGVSYYYYWGQGTLVTVSS |
| VH domain DNA | GAAGTGCAGCTGGTGCAGTCCGGAGCCGAAGTCAAGAGGCCT |
| (SEQ ID NO: 123) | GGAGCGTCCGTGAAGGTGTCCTGCAAAGCCTCAGGATACACCT |
| | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGC |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 50) | LIYEASNLESGVPSRFSGSGSGTEFTLTISSLQPEDFATYYCQQSY |
| | SWPRTFGQGTKVEIKRTVAAPSVFIFPPSDEQLKSGTASVVCLLNN |
| | FYPREAKVQWKVDNALQSGNSQESVTEQDSKDSTYSLSSTLTLSK |
| | ADYEKHKVYACEVTHQGLSSPVTKSFNRGEC |
| Light chain DNA | GACATTCAGATGACCCAGTCCCCGAGCACGCTGTCCGCAAGCG |
| (SEQ ID NO: 124) | TGGGGGACAGAGTGACCATCACTTGCCGCGCCTCACAATCCAT |
| | CAGCGGACGCTTGGCCTGGTACCAGCAGAAGCCCGGAAAGGC |
| | CCCAAACCTTCTGATCTACGAAGCCTCGAACCTGGAGTCAGGC |
| | GTCCCTTCGCGGTTCTCTGGCTCCGGTTCCGGAACTGAGTTCA |
| | CCCTCACCATCTCGTCCCTGCAACCGGAAGATTTCGCCACCTAC |
| | TACTGCCAACAGTCGTACTCCTGGCCCCGGACATTCGGACAGG |
| | GAACCAAAGTCGAGATTAAGCGGACTGTGGCGGCTCCTAGCGT |
| | GTTCATCTTTCCCCCGTCCGACGAACAGCTGAAGTCCGGTACC |
| | GCTAGCGTGGTCTGTCTCCTGAACAATTTCTACCCGCGCGAAGC |
| | TAAGGTCCAGTGGAAGGTCGACAACGCGCTGCAGTCCGGAAAC |
| | AGCCAGGAGTCAGTGACCGAGCAGGACTCCAAGGATTCCACTT |
| | ATTCCCTGTCCTCCACCCTGACTTTGAGCAAGGCCGACTACGAG |
| | AAGCACAAAGTGTACGCCTGCGAAGTGACCCATCAAGGGCTTT |
| | CGTCGCCCGTGACCAAGAGCTTCAACCGGGGCGAATGC |
| VL domain AA | DIQMTQSPSTLSASVGDRVTITCRASQSISGRLAWYQQKPGKAPNL |
| (SEQ ID NO: 30) | LIYEASNLESGVPSRFSGSGSGTEFTLTISSLQPEDFATYYCQQSY |
| | SWPRTFGQGTKVEIK |
| VL domain DNA | GACATTCAGATGACCCAGTCCCCGAGCACGCTGTCCGCAAGCG |
| (SEQ ID NO: 125) | TGGGGGACAGAGTGACCATCACTTGCCGCGCCTCACAATCCAT |
| | CAGCGGACGCTTGGCCTGGTACCAGCAGAAGCCCGGAAAGGC |
| | CCCAAACCTTCTGATCTACGAAGCCTCGAACCTGGAGTCAGGC |
| | GTCCCTTCGCGGTTCTCTGGCTCCGGTTCCGGAACTGAGTTCA |
| | CCCTCACCATCTCGTCCCTGCAACCGGAAGATTTCGCCACCTAC |
| | TACTGCCAACAGTCGTACTCCTGGCCCCGGACATTCGGACAGG |
| | GAACCAAAGTCGAGATTAAG |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EAS |
| (SEQ ID NO: 71) | |
| LCDR2 (AA) Kabat) | EASNLES |
| (SEQ ID NO: 20) | |
| LCDR3 (AA) (IMGT) | QQSYSWPRT |
| (SEQ ID NO: 21) | |
| LCDR3 (AA) (Kabat) | QQSYSWPRT |
| (SEQ ID NO: 21) | |

Amino acid and cDNA sequences of heavy and light chains and
variable domains of G1AA/lambdav3 and amino acid sequence of CDRs

| | |
|---|---|
| Heavy chain AA | EVQLVQSGAEVKRPGASVKVSCKASGYTFTSYGISWVRQAPGQG |
| (without LALA) | LEWMGWISAYSGGTNYAQKLQGRVTMTTDTSTSTAYMELRSLRS |
| (SEQ ID NO: 126) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (without LALA) | GGAGCGTCCGTGAAGGTGTCCTGCAAAGCCTCAGGATACACCT |
| (SEQ ID NO: 127) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAACTGCTGGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 61) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| Heavy chain DNA | GAAGTGCAGCTGGTGCAGTCCGGAGCCGAAGTCAAGAGGCCT |
| (with LALA) | GGAGCGTCCGTGAAGGTGTCCTGCAAAGCCTCAGGATACACCT |
| (SEQ ID NO: 128) | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGCGCTAGCACTAAGGGCCCGT |
| | CGGTGTTCCCGCTGGCCCCATCGTCCAAGAGCACATCAGGGGG |
| | TACCGCCGCCCTGGGCTGCCTTGTGAAGGATTACTTTCCCGAG |
| | CCCGTCACAGTGTCCTGGAACAGCGGAGCCCTGACCTCCGGAG |
| | TGCATACTTTCCCGGCTGTGCTTCAGTCCTCTGGCCTGTACTCA |
| | TTGTCCTCCGTGGTCACCGTCCCTTCGTCCTCCCTGGGCACCC |
| | AGACCTATATCTGTAATGTCAACCATAAGCCCTCGAACACCAAG |
| | GTCGACAAGAAGGTCGAGCCGAAGTCGTGCGACAAGACTCACA |
| | CTTGCCCGCCTTGCCCAGCCCCGGAAGCTGCCGGTGGTCCTTC |
| | GGTGTTCCTCTTCCCGCCCAAGCCGAAGGATACCCTGATGATCT |
| | CACGGACCCCCGAAGTGACCTGTGTGGTGGTGGACGTGTCCCA |
| | CGAGGACCCGGAAGTGAAATTCAATTGGTACGTGGATGGAGTG |
| | GAAGTGCACAACGCCAAGACCAAGCCACGGGAAGAACAGTACA |
| | ACTCTACCTACCGCGTGGTGTCCGTGCTCACTGTGCTGCACCAA |
| | GACTGGCTGAACGGGAAGGAGTACAAGTGCAAAGTGTCCAACA |
| | AGGCGCTGCCTGCCCCAATTGAGAAAACTATCTCGAAAGCCAA |
| | GGGACAGCCTCGAGAGCCTCAAGTGTACACCCTGCCTCCCTCT |
| | CGGGACGAGCTGACCAAGAACCAAGTCTCCCTGACCTGTCTGG |
| | TCAAGGGATTCTACCCATCGGATATCGCCGTGGAATGGGAAAG |
| | CAACGGACAGCCCGAGAACAACTACAAGACGACTCCGCCCGTG |
| | CTGGATTCCGACGGGAGCTTCTTCTTGTACTCCAAGCTGACCGT |
| | CGACAAGAGCAGATGGCAGCAGGGAAACGTGTTCTCCTGCTCC |
| | GTGATGCATGAGGCGCTGCACAACCACTACACTCAGAAGAGCT |
| | TGTCCCTGTCGCCCGGA |
| VH domain AA | EVQLVQSGAEVKRPGASVKVSCKASGYTFTSYGISWVRQAPGQG |
| (SEQ ID NO: 41) | LEWMGWISAYSGGTNYAQKLQGRVTMTTDTSTSTAYMELRSLRS |
| | DDTAVYYCARDLFPTIFGVSYYYYWGQGTLVTVSS |
| VH domain DNA | GAAGTGCAGCTGGTGCAGTCCGGAGCCGAAGTCAAGAGGCCT |
| (SEQ ID NO: 129) | GGAGCGTCCGTGAAGGTGTCCTGCAAAGCCTCAGGATACACCT |
| | TCACTTCGTACGGGATTTCCTGGGTCCGCCAAGCACCGGGTCA |
| | AGGCTTGGAGTGGATGGGATGGATCAGCGCGTATTCCGGGGGA |
| | ACCAACTACGCTCAAAAGCTGCAGGGTCGCGTGACCATGACCA |
| | CCGATACCTCCACCTCAACGGCCTACATGGAACTGAGATCTCTG |
| | CGGAGCGACGACACTGCCGTGTACTACTGTGCCCGGGACCTGT |
| | TCCCCACTATCTTCGGAGTGTCGTACTACTACTACTGGGGCCAG |
| | GGGACTCTCGTGACCGTGTCGAGC |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) (Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | QSALTQPASVSGSPGQSITISCTGTSSDVGGYNYVSWYQQFPGKA |
| (SEQ ID NO: 62) | PKLMIFEVTNRPSGVSDRFSGSKSDNTASLTISGLQAEDEAEYYCS |
| | SFKRGSTLVVFGGGTKLTVLGQPAAAPSVTLFPPSSEELQANKATL |
| | VCLISDFYPGAVTVAWKADSSPVKAGVETTTPSKQSNNKYAASSYL |
| | SLTPEQWKSHRSYSCQVTHEGSTVEKTVAPTECS |
| Light chain DNA | CAGTCGGCCCTTACTCAACCCGCGTCAGTCTCCGGTAGCCCCG |
| (SEQ ID NO: 130) | GACAGTCCATCACGATTTCGTGCACCGGAACCAGCAGCGATGT |
| | CGGGGGATACAACTACGTGTCCTGGTACCAGCAGTTCCCGGGA |
| | AAGGCCCCTAAGCTGATGATCTTCGAAGTCACTAACAGACCTTC |
| | CGGAGTGTCGGACCGGTTCTCCGGCTCCAAGTCCGACAACACT |
| | GCGAGCCTGACCATCTCGGGCCTGCAAGCCGAGGACGAAGCC |
| | GAGTACTACTGTAGCTCATTCAAGCGCGGTTCCACCCTCGTGGT |
| | GTTCGGCGGTGGCACTAAGCTCACCGTGCTGGGACAGCCAGCC |
| | GCAGCTCCTAGCGTGACCTTGTTCCCCCCGTCGAGCGAAGAAC |
| | TGCAGGCCAACAAGGCCACCCTCGTCTGCCTGATCTCCGACTT |
| | CTACCCTGGGGCCGTGACTGTGGCTTGGAAGGCCGATTCAAGC |
| | CCAGTGAAAGCCGGAGTGGAAACCACCACTCCGTCCAAGCAGT |
| | CGAACAATAAGTATGCCGCGTCCTCCTACCTGTCGCTGACCCC |
| | GGAGCAGTGGAAGTCCCATCGGTCCTACTCCTGCCAAGTCACC |
| | CACGAAGGGTCCACTGTGGAGAAAACAGTGGCTCCCACCGAGT |
| | GCTCT |
| VL domain AA | QSALTQPASVSGSPGQSITISCTGTSSDVGGYNYVSWYQQFPGKA |
| (SEQ ID NO: 42) | PKLMIFEVTNRPSGVSDRFSGSKSDNTASLTISGLQAEDEAEYYCS |
| | SFKRGSTLVVFGGGTKLTVL |
| VL domain DNA | CAGTCGGCCCTTACTCAACCCGCGTCAGTCTCCGGTAGCCCCG |
| (SEQ ID NO: 131) | GACAGTCCATCACGATTTCGTGCACCGGAACCAGCAGCGATGT |
| | CGGGGGATACAACTACGTGTCCTGGTACCAGCAGTTCCCGGGA |
| | AAGGCCCCTAAGCTGATGATCTTCGAAGTCACTAACAGACCTTC |
| | CGGAGTGTCGGACCGGTTCTCCGGCTCCAAGTCCGACAACACT |
| | GCGAGCCTGACCATCTCGGGCCTGCAAGCCGAGGACGAAGCC |
| | GAGTACTACTGTAGCTCATTCAAGCGCGGTTCCACCCTCGTGGT |
| | GTTCGGCGGTGGCACTAAGCTCACCGTGCTGGGA |
| LCDR1 (AA) (IMGT) | SSDVGGYNY |
| (SEQ ID NO: 132) | |
| LCDR1 (AA) (Kabat) | TGTSSDVGGYNYVS |
| (SEQ ID NO: 25) | |
| LCDR2 (AA) (IMGT) | EVT |
| (SEQ ID NO: 77) | |
| LCDR2 (AA) (Kabat) | EVTNRPS |
| (SEQ ID NO: 13) | |
| LCDR3 (AA) (IMGT) | SSFKRGSTLVV |
| (SEQ ID NO: 14) | |
| LCDR3 (AA) (Kabat) | SSFKRGSTLVV |
| (SEQ ID NO: 14) | |

Amino acid sequence of heavy and light chains, variable
domains and CDRs of G1/929_01_A07

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 133) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 134) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYGGST |
| (SEQ ID NO: 135) | |
| HCDR2 (AA) (Kabat) | WISAYGGSTNYAQKLQG |
| (SEQ ID NO: 136) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 137) | LIYEASNLESGVPSRFSGSGSGTEFTLTISSLQPEDFATYYCQQSY |
| | STPRVTFGQGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 138) | LIYEASNLESGVPSRFSGSGSGTEFTLTISSLQPEDFATYYCQQSY |
| | STPRVTFGQGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EAS |
| (SEQ ID NO: 71) | |
| LCDR2 (AA) (Kabat) | EASNLES |
| (SEQ ID NO: 20) | |
| LCDR3 (AA) (IMGT) | QQSYSTPRVT |
| (SEQ ID NO: 22) | |
| LCDR3 (AA) (Kabat) | QQSYSTPRVT |
| (SEQ ID NO: 22) | |

Amino acid sequence of heavy and light chains, variable
domains and CDRs of G1/929_01_A08
(This clone is the same as G1AA/E05v2 but
without the LALA mutation.)

| | |
|---|---|
| Heavy chain AA | EVQLVQSGAEVKRPGASVKVSCKASGYTFTSYGISWVRQAPGQG |
| (without LALA) | LEWMGWISAYSGGTNYAQKLQGRVTMTTDTSTSTAYMELRSLRS |
| (SEQ ID NO: 139) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 140) | LEWMGWISAYSGGTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 141) | LIYEASNLESGVPSRFSGSGSGTEFTLTISSLQPEDFATYYCQQSY |
| | STPRVTFGQGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 142) | LIYEASNLESGVPSRFSGSGSGTEFTLTISSLQPEDFATYYCQQSY |
| | STPRVTFGQGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EAS |
| (SEQ ID NO: 71) | |
| LCDR2 (AA) (Kabat) | EASNLES |
| (SEQ ID NO: 20) | |
| LCDR3 (AA) (IMGT) | QQSYSTPRVT |
| (SEQ ID NO: 22) | |
| LCDR3 (AA) (Kabat) | QQSYSTPRVT |
| (SEQ ID NO: 22) | |

Amino acid sequence of heavy and light chains, variable domains
and CDRs of G1/929_01_A09

| | |
|---|---|
| Heavy chain AA | EVQLVQSGAEVKRPGASVKVSCKASGYTFTSYGISWVRQAPGQG |
| (without LALA) | LEWMGWISAYSGNANYAQKLQGRVTMTTDTSTSTAYMELRSLRS |
| (SEQ ID NO: 143) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 144) | LEWMGWISAYSGNANYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGNA |
| (SEQ ID NO: 145) | |
| HCDR2 (AA) (Kabat) | WISAYSGNANYAQKLQG |
| (SEQ ID NO: 146) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 147) | LIYEASNLESGVPSRFSGSGSGTEFTLTISSLQPEDFATYYCQQSY |
| | STPRVTFGQGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 148) | LIYEASNLESGVPSRFSGSGSGTEFTLTISSLQPEDFATYYCQQSY |
| | STPRVTFGQGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EAS |
| (SEQ ID NO: 71) | |
| LCDR2 (AA) (Kabat) | EASNLES |
| (SEQ ID NO: 20) | |
| LCDR3 (AA) (IMGT) | QQSYSTPRVT |
| (SEQ ID NO: 22) | |
| LCDR3 (AA) (Kabat) | QQSYSTPRVT |
| (SEQ ID NO: 22) | |

Amino acid sequence of heavy and light chains, variable
domains and CDRs of G1/929_01_A01

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 149) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYPFTSYGISWVRQAPGQG |
| (SEQ ID NO: 150) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYPFTSYG |
| (SEQ ID NO: 102) | |
| HCDR1 (AA) (Kabat) | SYGIS |
| (SEQ ID NO: 1) | |
| HCDR2 (AA) (IMGT) | ISAYGGST |
| (SEQ ID NO: 135) | |
| HCDR2 (AA) (Kabat) | WISAYGGSTNYAQKLQG |
| (SEQ ID NO: 136) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVRDRVIITCRASQSIGNRLAWYQHKPGKAPKL |
| (SEQ ID NO: 151) | LIYEASTSETGVPSRFSGSGSGTDFTLTISSLQPEDFATYYCQQSY |
| | STPYTFGQGTKLEIKRTAAAAPSVFIFPPSDEQLKSGTASVVCLLNN |
| | FYPREAKVQWKVDNALQSGNSQESVTEQDSKDSTYSLSSTLTLSK |
| | ADYEKHKLYACEVTHQGLSSPVTKSFNRGEC |
| VL domain AA | DIQMTQSPSTLSASVRDRVIITCRASQSIGNRLAWYQHKPGKAPKL |
| (SEQ ID NO: 152) | LIYEASTSETGVPSRFSGSGSGTDFTLTISSLQPEDFATYYCQQSY |
| | STPYTFGQGTKLEIK |
| LCDR1 (AA) (IMGT) | QSIGNR |
| (SEQ ID NO: 103) | |
| LCDR1 (AA) (Kabat) | RASQSIGNRLA |
| (SEQ ID NO: 15) | |
| LCDR2 (AA) (IMGT) | EAS |
| (SEQ ID NO: 71) | |
| LCDR2 (AA) (Kabat) | EASTSET |
| (SEQ ID NO: 16) | |
| LCDR3 (AA) (IMGT) | QQSYSTPYT |
| (SEQ ID NO: 17) | |
| LCDR3 (AA) (Kabat) | QQSYSTPYT |
| (SEQ ID NO: 17) | |

Amino acid sequence of heavy and light chains, variable domains
and CDRs of G1/929_01_A02

| | |
|---|---|
| Heavy chain AA | EVQLVQSGAEVKRPGASVKVSCKASGYTFTSYGISWVRQAPGQG |
| (without LALA) | LEWMGWISAYSGGTNYAQKLQGRVTMTTDTSTSTAYMELRSLRS |
| (SEQ ID NO: 153) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYPFTSYGISWVRQAPGQG |
| (SEQ ID NO: 154) | LEWMGWISAYSGGTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYPFTSYG |
| (SEQ ID NO: 102) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVRDRVIITCRASQSIGNRLAWYQHKPGKAPKL |
| (SEQ ID NO: 155) | LIYEASTSETGVPSRFSGSGSGTDFTLTISSLQPEDFATYYCQQSY |
| | STPYTFGQGTKLEIKRTAAAAPSVFIFPPSDEQLKSGTASVVCLLNN |
| | FYPREAKVQWKVDNALQSGNSQESVTEQDSKDSTYSLSSTLTLSK |
| | ADYEKHKLYACEVTHQGLSSPVTKSFNRGEC |
| VL domain AA | DIQMTQSPSTLSASVRDRVIITCRASQSIGNRLAWYQHKPGKAPKL |
| (SEQ ID NO: 156) | LIYEASTSETGVPSRFSGSGSGTDFTLTISSLQPEDFATYYCQQSY |
| | STPYTFGQGTKLEIK |
| LCDR1 (AA) (IMGT) | QSIGNR |
| (SEQ ID NO: 103) | |
| LCDR1 (AA) (Kabat) | RASQSIGNRLA |
| (SEQ ID NO: 15) | |
| LCDR2 (AA) (IMGT) | EAS |
| (SEQ ID NO: 71) | |
| LCDR2 (AA) (Kabat) | EASTSET |
| (SEQ ID NO: 16) | |
| LCDR3 (AA) (IMGT) | QQSYSTPYT |
| (SEQ ID NO: 17) | |
| LCDR3 (AA) (Kabat) | QQSYSTPYT |
| (SEQ ID NO: 17) | |

Amino acid sequence of heavy and light chains, variable domains
and CDRs of G1/929_01_A03

| | |
|---|---|
| Heavy chain AA | EVQLVQSGAEVKRPGASVKVSCKASGYTFTSYGISWVRQAPGQG |
| (without LALA) | LEWMGWISAYSGNANYAQKLQGRVTMTTDTSTSTAYMELRSLRS |
| (SEQ ID NO: 157) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYPFTSYGISWVRQAPGQG |
| (SEQ ID NO: 158) | LEWMGWISAYSGNANYAQKLQGRVTMTTDTSTSTAYMELRSLRS |
| | DDTAVYYCARDLFPTIFGVSYYYYWGQGTLVTVSS |
| HCDR1 (AA) (IMGT) | GYPFTSYG |
| (SEQ ID NO: 102) | |
| HCDR1 (AA) (Kabat) | SYGIS |
| (SEQ ID NO: 1) | |
| HCDR2 (AA) (IMGT) | ISAYSGNA |
| (SEQ ID NO: 145) | |
| HCDR2 (AA) (Kabat) | WISAYSGNANYAQKLQG |
| (SEQ ID NO: 146) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVRDRVIITCRASQSIGNRLAWYQHKPGKAPKL |
| (SEQ ID NO: 159) | LIYEASTSETGVPSRFSGSGSGTDFTLTISSLQPEDFATYYCQQSY |
| | STPYTFGQGTKLEIKRTAAAAPSVFIFPPSDEQLKSGTASVVCLLNN |
| | FYPREAKVQWKVDNALQSGNSQESVTEQDSKDSTYSLSSTLTLSK |
| | ADYEKHKLYACEVTHQGLSSPVTKSFNRGEC |
| VL domain AA | DIQMTQSPSTLSASVRDRVIITCRASQSIGNRLAWYQHKPGKAPKL |
| (SEQ ID NO: 160) | LIYEASTSETGVPSRFSGSGSGTDFTLTISSLQPEDFATYYCQQSY |
| | STPYTFGQGTKLEIK |
| LCDR1 (AA) (IMGT) | QSIGNR |
| (SEQ ID NO: 103) | |
| LCDR1 (AA) (Kabat) | RASQSIGNRLA |
| (SEQ ID NO: 15) | |
| LCDR2 (AA) (IMGT) | EAS |
| (SEQ ID NO: 71) | |
| LCDR2 (AA) (Kabat) | EASTSET |
| (SEQ ID NO: 16) | |
| LCDR3 (AA) (IMGT) | QQSYSTPYT |
| (SEQ ID NO: 17) | |
| LCDR3 (AA) (Kabat) | QQSYSTPYT |
| (SEQ ID NO: 17) | |

Amino acid sequence of heavy and light chains,
variable domains and CDRs of G1/929_01_A10

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 161) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 162) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYGGST |
| (SEQ ID NO: 135) | |
| HCDR2 (AA) (Kabat) | WISAYGGSTNYAQKLQG |
| (SEQ ID NO: 136) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 163) | LIYEASNLESGVPSRFSGSGSGTEFTLTISSLQPEDFATYYCQQSY |
| | SWPRTFGQGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 164) | LIYEASNLESGVPSRFSGSGSGTEFTLTISSLQPEDFATYYCQQSY |
| | SWPRTFGQGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EASN |
| (SEQ ID NO: 106) | |
| LCDR2 (AA) (Kabat) | EASNLES |
| (SEQ ID NO: 20) | |
| LCDR3 (AA) (IMGT) | QQSYSWPRT |
| (SEQ ID NO: 21) | |
| LCDR3 (AA) (Kabat) | QQSYSWPRT |
| (SEQ ID NO: 21) | |

Amino acid sequence of heavy and light chains,
variable domains and CDRs of G1/929_01_A11
(This clone is the same as G1AA/G12v2 but
without the LALA mutation.)

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 165) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 29) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) (Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 166) | LIYEASNLESGVPSRFSGSGSGTEFTLTISSLQPEDFATYYCQQSY |
| | SWPRTFGQGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 167) | LIYEASNLESGVPSRFSGSGSGTEFTLTISSLQPEDFATYYCQQSY |
| | SWPRTFGQGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EASN |
| (SEQ ID NO: 106) | |
| LCDR2 (AA) (Kabat) | EASNLES |
| (SEQ ID NO: 20) | |
| LCDR3 (AA) (IMGT) | QQSYSWPRT |
| (SEQ ID NO: 21) | |
| LCDR3 (AA) (Kabat) | QQSYSWPRT |
| (SEQ ID NO: 21) | |

Amino acid sequence of heavy and light chains,
variable domains and CDRs of G1/929_01_A12

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 168) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 169) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGNA |
| (SEQ ID NO: 145) | |
| HCDR2 (AA) (Kabat) | WISAYSGNANYAQKLQG |
| (SEQ ID NO: 146) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 170) | LIYEASNLESGVPSRFSGSGSGTEFTLTISSLQPEDFATYYCQQSY |
| | SWPRTFGQGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 171) | LIYEASNLESGVPSRFSGSGSGTEFTLTISSLQPEDFATYYCQQSY |
| | SWPRTFGQGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EASN |
| (SEQ ID NO: 106) | |
| LCDR2 (AA) (Kabat) | EASNLES |
| (SEQ ID NO: 20) | |
| LCDR3 (AA) (IMGT) | QQSYSWPRT |
| (SEQ ID NO: 21) | |
| LCDR3 (AA) (Kabat) | QQSYSWPRT |
| (SEQ ID NO: 21) | |

Amino acid sequence of heavy and light chains, variable domains
and CDRs of G1/929_01_A04

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 172) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 173) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYGGST |
| (SEQ ID NO: 135) | |
| HCDR2 (AA) (Kabat) | WISAYGGSTNYAQKLQG |
| (SEQ ID NO: 136) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 174) | LIYEASNLESGVPSRFSGSGSGTEFTLTINSLQPDDFATYYCQQAN |
| | TFPRVSFGGGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 175) | LIYEASNLESGVPSRFSGSGSGTEFTLTINSLQPDDFATYYCQQAN |
| | TFPRVSFGGGTKVEIK |
| LCDR1 (AA) (IMGT | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EASN |
| (SEQ ID NO: 106) | |
| LCDR2 (AA) (Kabat) | EASNLES |
| (SEQ ID NO: 20) | |
| LCDR3 (AA) (IMGT) | QQANTFPRVS |
| (SEQ ID NO: 24) | |
| LCDR3 (AA) (Kabat) | QQANTFPRVS |
| (SEQ ID NO: 24) | |

Amino acid sequence of heavy and light chains, variable domains
and CDRs of G1/929_01_A05

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 176) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 177) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGGT |
| (SEQ ID NO: 111) | |
| HCDR2 (AA) (Kabat) | WISAYSGGTNYAQKLQG |
| (SEQ ID NO: 18) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 178) | LIYEASNLESGVPSRFSGSGSGTEFTLTINSLQPDDFATYYCQQAN |
| | TFPRVSFGGGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 179) | LIYEASNLESGVPSRFSGSGSGTEFTLTINSLQPDDFATYYCQQAN |
| | TFPRVSFGGGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT) | EASN |
| (SEQ ID NO: 106) | |
| LCDR2 (AA) (Kabat) | EASNLES |
| (SEQ ID NO: 20) | |
| LCDR3 (AA) (IMGT) | QQANTFPRVS |
| (SEQ ID NO: 24) | |
| LCDR3 (AA) (Kabat) | QQANTFPRVS |
| (SEQ ID NO: 24) | |

Amino acid sequence of heavy and light chains, variable domains
and CDRs of G1/929_01_A06

| | |
|---|---|
| Heavy chain AA | EVQLVQSGAEVKRPGASVKVSCKASGYTFTSYGISWVRQAPGQG |
| (without LALA) | LEWMGWISAYGGSTNYAQKLQGRVTMTTDTSTSTAYMELRSLRS |
| (SEQ ID NO: 180) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| VH domain AA | EVQLVQSGAEVKRPGASVKVSCKASGYTFTSYGISWVRQAPGQG |
| (SEQ ID NO: 181) | LEWMGWISAYGGSTNYAQKLQGRVTMTTDTSTSTAYMELRSLRS |
| | DDTAVYYCARDLFPTIFGVSYYYYWGQGTLVTVSS |
| HCDR1 (AA) (IMGT) | GYTFTSYG |
| (SEQ ID NO: 94) | |
| HCDR1 (AA) (Kabat) | SYGIS |
| (SEQ ID NO: 1) | |
| HCDR2 (AA) (IMGT) | ISAYSGNA |
| (SEQ ID NO: 145) | |
| HCDR2 (AA) (Kabat) | WISAYSGNANYAQKLQG |
| (SEQ ID NO: 146) | |
| HCDR3 (AA) (IMGT) | ARDLFPTIFGVSYYYY |
| (SEQ ID NO: 69) | |
| HCDR3 (AA) (Kabat) | DLFPTIFGVSYYYY |
| (SEQ ID NO: 3) | |
| Light chain AA | DIQMTQSPSTLSASVGDRVTITCRASQSISGRLAWYQQKPGKAPNL |
| (SEQ ID NO: 182) | LIYEASNLESGVPSRFSGSGSGTEFTLTINSLQPDDFATYYCQQAN |
| | TFPRVSFGGGTKVEIKRTAAAAPSVFIFPPSDEQLKSGTASVVCLLN |
| | NFYPREAKVQWKVDNALQSGNSQESVTEQDSKDSTYSLSSTLTLS |
| | KADYEKHKLYACEVTHQGLSSPVTKSFNRGEC |
| VL domain AA | DIQMTQSPSTLSASVGDRVTITCRASQSISGRLAWYQQKPGKAPNL |
| (SEQ ID NO: 183) | LIYEASNLESGVPSRFSGSGSGTEFTLTINSLQPDDFATYYCQQAN |
| | TFPRVSFGGGTKVEIK |
| LCDR1 (AA) (IMGT) | QSISGR |
| (SEQ ID NO: 100) | |
| LCDR1 (AA) (Kabat) | RASQSISGRLA |
| (SEQ ID NO: 19) | |
| LCDR2 (AA) (IMGT | EASN |
| (SEQ ID NO: 106) | |
| LCDR2 (AA) (Kabat) | EASNLES |
| (SEQ ID NO: 20) | |
| LCDR3 (AA) (IMGT) | QQANTFPRVS |
| (SEQ ID NO: 24) | |
| LCDR3 (AA) (Kabat) | QQANTFPRVS |
| (SEQ ID NO: 24) | |

mAbs Tested in mAb² Format

### Notes:

i. In the heavy chain sequence, variable domain in italics and, where applicable, location of LALA mutation in bold and underlined.
ii. In the light chain sequence, variable domain shown in italics.

Amino acid sequence of heavy and light chain of FS22-172-
003AA/E05v2 mAb²

| | |
|---|---|
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 89) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELPYIIPPYNQVSLTCLVKGFYPSDIAVEWESNGQPENNYKTTPPVL |
| | DSDGSFFLYSKLTVGADRWLEGNVFSCSVMHEALHNHYTQKSLSL |
| | SPG |
| Light chain AA | DIQMTQSPSTLSASVGDRVTITCRASQSISGRLAWYQQKPGKAPNL |
| (SEQ ID NO: 90) | LIYEASNLESGVPSRFSGSGSGTEFTLTISSLQPEDFATYYCQQSYS |
| | TPRVTFGQGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |

Amino acid sequence of heavy and light chain of FS22-172-
003AA/E12v2 mAb²

| | |
|---|---|
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 85) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELPYIIPPYNQVSLTCLVKGFYPSDIAVEWESNGQPENNYKTTPPVL |
| | DSDGSFFLYSKLTVGADRWLEGNVFSCSVMHEALHNHYTQKSLSL |
| | SPG |
| Light chain AA | DIQMTQSPSTLSASVGDRVTITCRASQSISGRLAWYQQKPGKAPNL |
| (SEQ ID NO: 86) | LIYEASNLESGVPSRFSGSGSGTEFTLTISSLQPEDFATYYCQQSYS |
| | TPRVTFGQGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |

Amino acid sequence of heavy and light chain of FS22-172-
003AA/G12v2 mAb²

| | |
|---|---|
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 87) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELPYIIPPYNQVSLTCLVKGFYPSDIAVEWESNGQPENNYKTTPPVL |
| | DSDGSFFLYSKLTVGADRWLEGNVFSCSVMHEALHNHYTQKSLSL |
| | SPG |
| Light chain AA | DIQMTQSPSTLSASVGDRVTITCRASQSISGRLAWYQQKPGKAPNL |
| (SEQ ID NO: 88) | LIYEASNLESGVPSRFSGSGSGTEFTLTISSLQPEDFATYYCQQSYS |
| | TPRVTFGQGTKVEIKRTVAAPSVFIFPPSDEQLKSGTASVVCLLNNF |
| | YPREAKVQWKVDNALQSGNSQESVTEQDSKDSTYSLSSTLTLSKA |
| | DYEKHKVYACEVTHQGLSSPVTKSFNRGEC |

Amino acid sequence of heavy and light chain of FS22-172-
003AA/lambdav3 mAb²

| | |
|---|---|
| Heavy chain AA | EVQLVQSGAEVKRPGASVKVSCKASGYTFTSYGISWVRQAPGQG |
| (with LALA) | LEWMGWISAYSGGTNYAQKLQGRVTMTTDTSTSTAYMELRSLRS |
| (SEQ ID NO: 91) | DDTAVYYCARDLFPTIFGVSYYYYWGQGTLVTVSSASTKGPSVFPL |
| | APSSKSTSGGTAALGCLVKDYFPEPVTVSWNSGALTSGVHTFPAV |
| | LQSSGLYSLSSVVTVPSSSLGTQTYICNVNHKPSNTKVDKKVEPKS |
| | CDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLH |
| | QDWLNGKEYKCKVSNKALPAPIEKTISKAKGQPREPQVYTLPPSRD |
| | ELPYIIPPYNQVSLTCLVKGFYPSDIAVEWESNGQPENNYKTTPPVL |
| | DSDGSFFLYSKLTVGADRWLEGNVFSCSVMHEALHNHYTQKSLSL |
| | SPG |
| Light chain AA | QSALTQPASVSGSPGQSITISCTGTSSDVGGYNYVSWYQQFPGKA |
| (SEQ ID NO: 92) | PKLMIFEVTNRPSGVSDRFSGSKSDNTASLTISGLQAEDEAEYYCS |
| | SFKRGSTLVVFGGGTKLTVLGQPAAAPSVTLFPPSSEELQANKATL |
| | VCLISDFYPGAVTVAWKADSSPVKAGVETTTPSKQSNNKYAASSYL |
| | SLTPEQWKSHRSYSCQVTHEGSTVEKTVAPTECS |

PD-L1-rCd4-His

| | |
|---|---|
| Human | MRIFAVFIFMTYWHLLNAFTVTVPKDLYVVEYGSNMTIECKFPVEKQ |
| (SEQ ID NO: 79) | LDLAALIVYWEMEDKNIIQFVHGEEDLKVQHSSYRQRARLLKDQLS |
| | LGNAALQITDVKLQDAGVYRCMISYGGADYKRITVKVNAPYNKINQ |
| | RILVVDPVTSEHELTCQAEGYPKAEVIWTSSDHQVLSGKTTTTNSK |
| | REEKLFNVTSTLRINTTTNEIFYCTFRRLDPEENHTAELVIPELPLAH |
| | PPNERTAAATSITAYKSEGESAEFSFPLNLGEESLQGELRWKAEKA |
| | PSSQSWITFSLKNQKVSVQKSTSNPKFQLSETLPLTLQIPQVSLQFA |
| | GSGNLTLTLDRGILYQEVNLVVMKVTQPDSNTLTCEVMGPTSPKM |
| | RLILKQENQEARVSRQEKVIQVQAPEAGVWQCLLSEGEEVKMDSKI |
| | QVLSKGLNGSHHHHHH |
| Mouse | MRIFAGIIFTACCHLLRAFTITAPKDLYVVEYGSNVTMECRFPVEREL |
| (SEQ ID NO: 81) | DLLALVVYWEKEDEQVIQFVAGEEDLKPQHSNFRGRASLPKDQLL |
| | KGNAALQITDVKLQDAGVYCCIISYGGADYKRITLKVNAPYRKINQRI |
| | SVDPATSEHELICQAEGYPEAEVIWTNSDHQPVSGKRSVTTSRTE |
| | GMLLNVTSSLRVNATANDVFYCTFWRSQPGQNHTAELIIPELPATH |
| | PPQNRTAAATSITAYKSEGESAEFSFPLNLGEESLQGELRWKAEK |
| | APSSQSWITFSLKNQKVSVQKSTSNPKFQLSETLPLTLQIPQVSLQF |
| | AGSGNLTLTLDRGILYQEVNLVVMKVTQPDSNTLTCEVMGPTSPK |
| | MRLILKQENQEARVSRQEKVIQVQAPEAGVWQCLLSEGEEVKMDS |
| | KIQVLSKGLNGSHHHHHH |
| Cyno | MRIFAVFIFTIYWHLLNAFTVTVPKDLYVVEYGSNMTIECKFPVEKQL |
| (SEQ ID NO: 184) | DLTSLIVYWEMEDKNIIQFVHGEEDLKVQHSNYRQRAQLLKDQLSL |
| | GNAALRITDVKLQDAGVYRCMISYGGADYKRITVKVNAPYNKINQRI |
| | LVVDPVTSEHELTCQAEGYPKAEVIWTSSDHQVLSGKTTTTNSKRE |
| | EKLLNVTSTLRINTTANEIFYCIFRRLDPEENHTAELVIPELPLALPPN |
| | ERTAAATSITAYKSEGESAEFSFPLNLGEESLQGELRWKAEKAPSS |
| | QSWITFSLKNQKVSVQKSTSNPKFQLSETLPLTLQIPQVSLQFAGS |
| | GNLTLTLDRGILYQEVNLVVMKVTQPDSNTLTCEVMGPTSPKMRLI |
| | LKQENQEARVSRQEKVIQVQAPEAGVWQCLLSEGEEVKMDSKIQV |
| | LSKGLNGSHHHHHH |

PD-L1-Fc-His

| | |
|---|---|
| Human | MRIFAVFIFMTYWHLLNAFTVTVPKDLYVVEYGSNMTIECKFPVEKQ |
| (SEQ ID NO: 80) | LDLAALIVYWEMEDKNIIQFVHGEEDLKVQHSSYRQRARLLKDQLS |
| | LGNAALQITDVKLQDAGVYRCMISYGGADYKRITVKVNAPYNKINQ |
| | RILVVDPVTSEHELTCQAEGYPKAEVIWTSSDHQVLSGKTTTTNSK |
| | REEKLFNVTSTLRINTTTNEIFYCTFRRLDPEENHTAELVIPELPLAH |
| | PPNERTAAADKTHTCPPCPAPELLGGPSVFLFPPKPKDTLMISRTP |
| | EVTCVVVDVSHEDPEVKFNWYVDGVEVHNAKTKPREEQYNSTYR |
| | VVSVLTVLHQDWLNGKEYKCKVSNKALPAPIEKTISKAKGQPREPQ |
| | VYTLPPSRDELTKNQVSLTCLVKGFYPSDIAVEWESNGQPENNYKT |
| | TPPVLDSDGSFFLYSKLTVDKSRWQQGNVFSCSVMHEALHNHYT |
| | QKSLSLSPGKGSHHHHHH |
| Mouse | MRIFAGIIFTACCHLLRAFTITAPKDLYVVEYGSNVTMECRFPVEREL |
| (SEQ ID NO: 82) | DLLALVVYWEKEDEQVIQFVAGEEDLKPQHSNFRGRASLPKDQLL |
| | KGNAALQITDVKLQDAGVYCCIISYGGADYKRITLKVNAPYRKINQRI |
| | SVDPATSEHELICQAEGYPEAEVIWTNSDHQPVSGKRSVTTSRTE |
| | GMLLNVTSSLRVNATANDVFYCTFWRSQPGQNHTAELIIPELPATH |
| | PPQNRTAAADKTHTCPPCPAPELLGGPSVFLFPPKPKDTLMISRTP |
| | EVTCVVVDVSHEDPEVKFNWYVDGVEVHNAKTKPREEQYNSTYR |
| | VVSVLTVLHQDWLNGKEYKCKVSNKALPAPIEKTISKAKGQPREPQ |
| | VYTLPPSRDELTKNQVSLTCLVKGFYPSDIAVEWESNGQPENNYKT |
| | TPPVLDSDGSFFLYSKLTVDKSRWQQGNVFSCSVMHEALHNHYT |
| | QKSLSLSPGKGSHHHHHH |

Signal peptide (underlined)
Extracellular domain of PD-L1 (regular font)
Human lgG1 Fc (italics)
Junction between antigen and C-terminal fusion encoding a Notl restriction site (bold and underlined)
C-terminal hexahistidine tag (italics and underlined)

PD-L1

| | |
|---|---|
| Human | FTVTVPKDLYVVEYGSNMTIECKFPVEKQLDLAALIVYWEMEDKNII |
| (SEQ ID NO: 83) | QFVHGEEDLKVQHSSYRQRARLLKDQLSLGNAALQITDVKLQDAG |
| | VYRCMISYGGADYKRITVKVNAPYNKINQRILVVDPVTSEHELTCQA |
| | EGYPKAEVIWTSSDHQVLSGKTTTTNSKREEKLFNVTSTLRINTTTN |
| | EIFYCTFRRLDPEENHTAELVIPELPLAHPPNERTHLVILGAILLCLG |
| | VALTFIFRLRKGRMMDVKKCGIQDTNSKKQSDTHLEET |
| Mouse | FTITAPKDLYVVEYGSNVTMECRFPVERELDLLALVVYWEKEDEQV |
| (SEQ ID NO: 84) | IQFVAGEEDLKPQHSNFRGRASLPKDQLLKGNAALQITDVKLQDAG |
| | VYCCIISYGGADYKRITLKVNAPYRKINQRISVDPATSEHELICQAEG |
| | YPEAEVIWTNSDHQPVSGKRSVTTSRTEGMLLNVTSSLRVNATAN |
| | DVFYCTFWRSQPGQNHTAELIIPELPATHPPQNRTHWVLLGSILLF |
| | LIVVSTVLLFLRKQVRMLDVEKCGVEDTSSKNRNDTQFEET |

Extracellular domain (italics)
Transmembrane and intracellular domains (bold)

1. An antibody or antigen-binding fragment thereof, capable of binding specifically to PD-L1, comprising a variable heavy (VH) domain comprising heavy chain complementarity determining regions (CDRs) HCDR1, HCDR2 and HCDR3, and a variable light (VL) domain comprising light chain CDRs LCDR1, LCDR2 and LCDR3, wherein the antibody or antigen-binding fragment thereof comprises an antigen-binding site comprising HCDR1, HCDR2, HCDR3, LCDR1, LCDR2 and LCDR3;
wherein the antigen-binding site comprises HCDR1, HCDR2, HCDR3, LCDR1, LCDR2 and LCDR3 set forth in:
(a) SEQ ID NO: 1, 18, 3, 15, 16 and 17;
(b) SEQ ID NO: 1, 18, 3, 19, 20 and 21;
(c) SEQ ID NO: 1, 18, 3, 19, 20 and 22;
(d) SEQ ID NO: 1, 23, 3, 15, 16, and 17;
(e) SEQ ID NO: 1, 23, 3, 19, 20 and 21;
(f) SEQ ID NO: 1, 23, 3, 19, 20 and 22;
(g) SEQ ID NO: 1, 23, 3, 19, 20 and 24;
(h) SEQ ID NO: 1, 18, 3, 25, 13 and 14;
(i) SEQ ID NO: 1, 23, 3, 26, 13 and 14; or
(j) SEQ ID NO: 1, 78, 3, 26, 13 and 14;
wherein the sequences are defined by Kabat nomenclature; and/or
wherein the antigen-binding site comprises HCDR1, HCDR2, HCDR3, LCDR1, LCDR2 and LCDR3 set forth in:
(a) SEQ ID NO: 102, 111, 69, 103, 71 and 17;
(b) SEQ ID NO: 94, 111, 69, 100, 71 and 21;
(c) SEQ ID NO: 94, 111, 69, 100, 71 and 22;
(d) SEQ ID NO: 102, 98, 69, 103, 71 and 17;
(e) SEQ ID NO: 94, 98, 69, 100, 77 and 21;
(f) SEQ ID NO: 94, 98, 69, 100, 71 and 22;
(g) SEQ ID NO: 94, 98, 69, 100, 106 and 24;
(h) SEQ ID NO: 94, 111, 69, 132, 77 and 14;
(i) SEQ ID NO: 94, 98, 69, 96, 77 and 14; or
(j) SEQ ID NO: 94, 95, 69, 96, 77 and 14;
wherein the sequences are defined by ImMunoGeneTics (IMGT) nomenclature.

2.-8. (canceled)

9. An antibody or antigen-binding fragment thereof according to claim 1, wherein the antigen-binding site comprises the VH domain and/or the VL domain set forth in:
(a) SEQ ID NO: 27 and 28, respectively;
(b) SEQ ID NO: 29 and 30, respectively;
(c) SEQ ID NO: 31 and 32, respectively;
(d) SEQ ID NO: 33 and 34, respectively;
(e) SEQ ID NO: 35 and 36, respectively;
(f) SEQ ID NO: 37 and 38, respectively;
(g) SEQ ID NO: 39 and 40, respectively;
(h) SEQ ID NO: 41 and 42, respectively;
(i) SEQ ID NO: 43 and 44, respectively; or
(j) SEQ ID NO: 45 and 46, respectively.

10. An antibody according to claim 1, wherein the antibody comprises the heavy chain and/or the light chain set forth in:
(a) SEQ ID NO: 47 and 48, respectively;
(b) SEQ ID NO: 49 and 50, respectively;
(c) SEQ ID NO: 51 and 52, respectively;
(d) SEQ ID NO: 53 and 54, respectively;
(e) SEQ ID NO: 55 and 56, respectively;
(f) SEQ ID NO: 57 and 58, respectively;
(g) SEQ ID NO: 59 and 60, respectively;
(h) SEQ ID NO: 61 and 62, respectively;
(i) SEQ ID NO: 63 and 64, respectively; or
(j) SEQ ID NO: 65 and 66, respectively.

11. An antibody or antigen-binding fragment thereof according to claim 1, comprising HCDR1, HCDR2, HCDR3, LCDR1, LCDR2 and LCDR3, defined according to the Kabat numbering scheme; HCDR1, HCDR2, HCDR3, LCDR1, LCDR2 and LCDR3, defined according to the IMGT numbering scheme; the VH domain and/or the VL domain; the Fab; and/or the light chain and/or the heavy chain of antibody G1AA/E12v2, G1AA/G12v2 or G1AA/E05v2.

12. An antibody or antigen-binding fragment thereof according to claim 1, comprising HCDR1, HCDR2, HCDR3 LCDR1, LCDR2 and LCDR3, defined according to the Kabat number scheme; HCDR1, HCDR2, HCDR3, LCDR1, LCDR2 and LCDR3, defined according to the IMGT numbering scheme; the VH domain and/or the VL, Fab, light chain and/or the heavy chain of antibody G1AA/E12v2 or G1/E12v2.

13.-18. (canceled)

19. An antibody or antigen-binding fragment thereof, according to claim 1, wherein the antibody or antigen-binding fragment thereof is a multispecific molecule comprising at least a second antigen-binding site.

20. An antibody or antigen-binding fragment thereof, according to claim 19, wherein the second antigen-binding site is located in a constant domain of the antibody or antigen-binding fragment.

21. (canceled)

22. An antibody or antigen-binding fragment thereof, according to claim 20, wherein the constant domain is a CH3 domain.

23.-25. (canceled)

26. An antibody or antigen-binding fragment thereof according to claim 1, wherein the antibody is an IgG1 or fragment thereof with a modified Fc region with reduced immune effector function.

27.-29. (canceled)

30. An antibody or antigen-binding fragment thereof, according to claim 19, wherein the second antigen-binding site binds to an inhibitory checkpoint molecule, costimulatory molecule or tumour-associated antigen.

31.-35. (canceled)

36. A nucleic acid molecule or set of nucleic acid molecules encoding an antibody or antigen-binding fragment thereof according to claim 1.

37.-42. (canceled)

43. A composition comprising the antibody or antigen-binding fragment thereof according to claim 1 and an excipient.

44. (canceled)

45. A method for treatment of a disease or disorder in a patient comprising administering to the patient a therapeutically-effective amount of an antibody or antigen-binding fragment thereof according to any of claim 1.

46.-52. (canceled)

53. An antibody or antigen-binding fragment thereof according to claim 19, wherein the antibody or antigen-binding fragment thereof is a bispecific molecule comprising at least a second antigen-binding site.

54. An antibody or antigen-binding fragment thereof according to claim 53, wherein the second antigen-binding site is located in a constant domain of the antibody or antigen-binding fragment.

55. An antibody or antigen-binding fragment thereof according to claim 54, wherein the constant domain is a CH3 domain.

56. An antibody or antigen-binding fragment thereof according to claim 55, wherein the second antigen-binding site binds to an inhibitory checkpoint molecule, costimulatory molecule or tumour-associated antigen.

57. A composition according to claim 43, wherein the composition is a pharmaceutical composition comprising the antibody or antigen-binding fragment thereof according to claim 1 and a pharmaceutically-acceptable excipient.

58. A pharmaceutical composition comprising the antibody or antigen-binding fragment thereof according to claim 56 and a pharmaceutically-acceptable excipient.

59. A method for treatment of a disease or disorder in a patient comprising administering to the patient a therapeutically-effective amount of an antibody or antigen-binding fragment thereof according to claim 56.
